SEQUENCE LISTING (18 sequences)

SEQ ID NO: 1 - DNA, 69 bp, Homo sapiens
atctaccagaacctgacccagcttaaagctgcagtgggtgagctctcagagaaatccaagctgcaggag

SEQ ID NO: 2 - DNA, 69 bp, Homo sapiens
atctaccaggagctgacccagctgaaggctgcagtgggtgagttgccagagaaatccaagctgcaggag

SEQ ID NO: 3 - DNA, 69 bp, Homo sapiens
atctaccaggagctgacccggctgaaggctgcagtgggtgagttgccagagaaatccaagctgcaggag

SEQ ID NO: 4 - DNA, 69 bp, Homo sapiens
atctaccaggagctgacggagctgaaggctgcagtgggtgagttgccagagaaatccaagctgcaggag

SEQ ID NO: 5 - DNA, 69 bp, Homo sapiens
atctaccaggagctgacccagctgaaggctgcagtgggtgagttgccagaccagtccaagcagcagcaa

SEQ ID NO: 6 - protein, 23 aa, Homo sapiens
IYQNLTQLKAAVGELSEKSKLQE

SEQ ID NO: 7 - protein, 23 aa, Homo sapiens
IYQELTQLKAAVGELPEKSKLQE

SEQ ID NO: 8 - protein, 23 aa, Homo sapiens
IYQELTRLKAAVGELPEKSKLQE

SEQ ID NO: 9 - protein, 23 aa, Homo sapiens
IYQELTELKAAVGELPEKSKLQE

SEQ ID NO: 10 - protein, 23 aa, Homo sapiens
IYQELTQLKAAVGELPDQSKQQQ

SEQ ID NO: 11 - protein, 23 aa, Homo sapiens
  MISC_FEATURE (8)..(22): X represents any amino acid
KAAVGELXEKSKXQEIYQELTXL

SEQ ID NO: 12 - protein, 307 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTRLKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 13 - protein, 330 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTRLKAAVGELPEKSKLQEIYQELTRLKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 14 - protein, 353 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 15 - protein, 399 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 16 - protein, 445 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 17 - DNA, 21 bp, Homo sapiens
tgtccaaggtccccagctccc

SEQ ID NO: 18 - DNA, 27 bp, Homo sapiens
gaactcaccaaatgcagtcttcaaatc

(19) United States
(12) Patent Application Publication
(10) Pub. No.: US 2006/0292151 A1
(43) Pub. Date: Dec. 28, 2006

(54) L-SIGN polymorphisms and methods involving use of same

(76) Inventors: Jason P. Gardner, Southport (GB); William C. Olson, Ossining, NY (US)

Correspondence Address: COOPER & DUNHAM, LLP, 1185 AVENUE OF THE AMERICAS, NEW YORK, NY, 10036, US

(21) Appl. No.: 11/093,117

(22) Filed: Mar. 28, 2005

Related U.S. Application Data
(60) Provisional application No. 60/556,725, filed on Mar. 26, 2004.

(51) Int. Cl. A61K 39/395; G01N 33/53; A61K 31/715; A61K 31/716

(52) U.S. Cl. 424/146.1; 435/7.1; 530/388.26; 536/123; 514/54

(57) ABSTRACT

This invention concerns methods for determining whether an agent preferentially binds to at least one allelic variant of L-SIGN. The invention is also directed to agents that preferentially bind to at least one allelic variant of L-SIGN. The invention also provides methods and agents for treating and preventing disorders associated with infection by pathogens, including hepatitis C virus, that bind to particular L-SIGN allelic variants. The invention further provides methods for predicting the resistance or susceptibility of a subject to pathogen infection.

This application claims the benefit of U.S. Provisional Application No. 60/556,725, filed Mar. 26, 2004, the contents of which are hereby incorporated by reference into this application.

Throughout this application, various publications are referenced in parentheses by author name and date. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein. However, the citation of a reference herein should not be construed as an acknowledgement that such reference is prior art to the present invention.

### BACKGROUND OF THE INVENTION

Hepatitis C virus (HCV) was first recognized in 1989. It infects the liver and is responsible for the majority of cases of non-A, non-B hepatitis (Lauer and Walker, 2001; Alter and Seef, 1993). Infections are typically chronic and lifelong; many infected individuals are healthy and unaffected for decades, whereas others develop chronic hepatitis or liver cirrhosis, the latter often leading to hepatocellular carcinoma (Fry and Flint, 1997; Lauer and Walker, 2001). While screening of the blood supply has drastically reduced new transmissions of the virus, there exists a large cohort of infected individuals who will require treatment in the coming decades. Some reports estimate that nearly 3% of the world's population or about 170 million people worldwide, including about 4 million people in the U.S.A., are infected with HCV (Anon, 1999; Cooper et al., 1999; Lechner et al., 2000).

HCV infection and its clinical sequelae are the leading causes of liver transplantation in the U.S.A. No vaccine is currently available, and the two licensed therapies, interferon alpha and ribavirin, which are both non-specific anti-viral agents with incompletely understood mechanisms of action, are only modestly efficacious (McHutchison et al., 1998). Thus, whereas the best long-term response rates are obtained with a combination of interferon alpha-2b and ribavirin, only a minority of subjects treated with this combination achieves the desired result of no detectable serum HCV RNA six months after stopping treatment (McHutchison et al., 1998). Moreover, these drugs exhibit severe, life-threatening toxicities, including neutropenia, hemolytic anemia and severe depression. There is therefore an urgent need for the development of new therapeutic approaches and agents to combat HCV infection.

The HCV genome is a 9.6 kb positive-sense, single-stranded RNA molecule that encodes a single polyprotein of ˜3000 amino acids (Rice, 1996) which is processed to generate at least ten proteins including structural and nonstructural proteins (Grakoui et al., 1993; Lauer and Walker, 2001). HCV isolates exhibit considerable sequence diversity and have been classified into six major genotypes (exhibiting less than 70% sequence identity), and further into subtypes (exhibiting 80-90% identity) (Yanagi et al., 1999). Genotype 1 (subtypes 1a and 1b) predominates in North America, Europe, and Japan but subtypes 2a, 2b and 2c are also common (Smith and Simmonds, 1998). The genetic variants found within an infected individual are termed quasispecies, and result from an interplay of random mutations introduced during viral replication and selective pressures within the host. There are no clear differences in pathology associated with the different genotypes.

The development of new treatments for HCV infection would be facilitated by a better understanding of how HCV attaches to and fuses with cell membranes and enters target cells. Viral entry into target cells is a particularly attractive target for anti-HCV therapy because entry inhibitors do not need to cross the plasma membrane nor be modified intracellularly. In addition, viral entry is generally a rate-limiting step that is mediated by conserved structures on the viral envelope and cell membrane. Consequently, inhibitors of viral entry can provide potent and durable suppression of viral replication.

HCV entry into host cells requires attachment of the viral particle to the cell surface, followed by fusion of the viral envelope with the cellular membrane. The HCV envelope glycoproteins, E1 and E2 are thought to be responsible for the binding of the virus to target cells. Supporting this view is the demonstration of fusion and entry of the TM domain of the vesicular stomatitis virus envelope glycoprotein (VSV G), mediated by linkage to the ectodomains of E1 and E2 (Lagging et al., 1998; Takikawa et al., 2000). In mammalian cell-based expression systems, the molecular weight of mature, full length E1 is ˜35 kD and that of E2 is ˜72 kD (Grakoui et al., 1993; Matsuura et al., 1994; Spaete et al., 1992). E1 and E2 form non-covalently associated heterodimers, hereinafter referred to as E1/E2, on the virus surface and undergo extensive posttranslational modification by N-linked glycosylation (Lauer and Walker, 2001).

A fundamental riddle of HCV infection has been the mechanism by which the virus targets the liver. Tissue and cellular tropisms of viruses are often regulated by one or more host receptors that mediate distinct functions such as viral attachment, internalization, fusion and trafficking (Doms and Moore, 2000; Sieczkarski and Whittaker, 2002). Glycosaminoglycans have been suggested to play a role in the nonspecific attachment of HCV to cells (Wunschmann et al., 2000), and various human cellular proteins have been implicated as putative receptors that mediate HCV entry into cells.

One such protein is CD81 (Jones et al., 2000). The recombinant soluble E2 ectodomain binds specifically and with high affinity (K_{d}≈10⁻⁸ M) to human and chimpanzee CD81, but not to CD81 from other species (Flint et al., 1999; Higginbottom et al., 2000; Petracca et al., 2000; Pileri et al., 1998). Further, it has recently been demonstrated that CD81 functions as a post-attachment entry coreceptor for HCV, and that other cellular factors act in concert with CD81 to mediate HCV binding and entry into hepatocytes (Cormier et al., 2004a). However, CD81 is expressed on numerous tissues outside of the liver, and thus CD81 tissue distribution cannot account for the restricted tropism of HCV to hepatocytes and perhaps certain lymphocytes.

Another putative cellular receptor for HCV is the low density lipoprotein receptor (LDL-R) (Jones et al., 2000). However, although the expression pattern of LDL-R is consistent with HCV tropism, studies to date have failed to demonstrate a direct interaction between LDL-R and the HCV envelope glycoproteins (Wunschmann et al., 2000). A broadly expressed lipoprotein binding receptor, human scavenger receptor class B type 1, has been shown to bind to E2 in vitro, but binding to virus was not demonstrated (Scarselli et al., 2002). Furthermore, the wide expression of this receptor on tissues other than liver does not explain the liver-specific tropism of HCV.

L-SIGN (liver/lymph node-specific intercellular adhesion molecule-3 (ICAM-3)-grabbing nonintegrin, also referred to as CD209L and DC-SIGNR, i.e., DC-SIGN Related; Genbank accession number AF245219) and DC-SIGN (dendritic cell-specific ICAM-3-grabbing nonintegrin, also referred to as CD209; Genbank accession number AF209479) are homologous (77% amino acid identity) type II membrane proteins characterized by a carboxy-terminal carbohydrate-recognition domain (CRD); an oligomerization or “neck” domain, encoded by Exon 4, which contains seven repeats of a 23-amino acid sequence; a single transmembrane-spanning domain of approximately 22 amino acids; and a short cytoplasmic domain. L-SIGN is highly expressed on liver sinusoidal endothelial cells (LSECs) (Pohlmann et al., 2001a; Bashirova et al., 2001; Soilleux et al., 2000), which are specialized nonmyeloid antigen-presenting cells involved in immune surveillance (Knolle and Gerken, 2000), and in lymph nodes but not on dendritic cells (Bashirova et al., 2001). In contrast, DC-SIGN is expressed at high levels on dendritic cells and is important for activation of resting T cells (Geijtenbeek et al., 2000). Both molecules are expressed on endometrium and placenta (Pohlmann et al., 2001; Soilleux et al., 2000; Bashirova et al., 2001; Geijtenbeek et al., 2000).

L-SIGN and DC-SIGN are C-type (calcium-dependent) lectins that possess all of the residues known to be required for binding of mannose. Since these C-type lectins typically bind high-mannose and related glycans on the surface of pathogens, the SIGN molecules act as receptors for certain viral and non-viral pathogens (van Kooyk and Geijtenbeek, 2003). For example, they bind the (human immunodeficiency virus type 1 (HIV-1) surface envelope glycoprotein gp120, which possesses high-mannose sugars, and this binding is inhibited by mannan (Soilleux et al., 2000; Bashirova et al., 2001; Geijtenbeek et al., 2000). Both DC-SIGN and L-SIGN bind infectious HIV-1 particles. However, the SIGN molecules do not act as conventional entry receptors for viruses such as HIV-1. Instead, SIGN-expressing cells capture virus and facilitate its delivery to, and trans-infection of, susceptible target cells (Pohlmann et al., 2001a, 2001b; Geijtenbeek et al., 2000; Soilleux et al., 2000; Bashirova et al., 2001). European patent applications EP 1046651 A1 and EP 1086137 A1 describe the use of DC-SIGN in compositions and methods for inhibiting HIV-1 infection. The entire contents of these applications are incorporated herein by reference.

Like DC-SIGN and L-SIGN, the lectin Galanthus nivalis (GNA lectin) from snowdrop bulbs avidly binds carbohydrates and glycoproteins possessing high-mannose structures. Notably, GNA lectin avidly binds HIV-1 envelope glycoproteins (Gilljam, 1993; Trkola et al., 1996) and captures the HCV envelope glycoproteins (Flint et al., 2000) which contain high-mannose carbohydrates.

L-SIGN is known to bind to a broad range of pathogens, including but not limited to human immunodeficiency virus, simian immunodeficiency virus, dengue virus, Ebola virus, Marburg virus, severe acute respiratory syndrome (SARS) coronavirus, Sindbis virus, and cytomegalovirus. Other non-viral pathogens that target the liver may also bind to L-SIGN, for example, sporozoites from Plasmodium spp., in addition to other pathogens not yet described to bind to L-SIGN but known to bind to the homologous DC-SIGN (for example, Aspergillus, Candida, Mycobacterium, Helicobacter, Leishmania and Schistosoma).

Of the pathogens known to bind L-SIGN, HCV is the most hepatotropic. Hepatocytes are the major target cells of HCV (Boisvert et al., 2001; Fournier et al., 1998; Ikeda et al., 1998), but the existence of extrahepatic reservoirs of HCV is indicated by the detection of viral RNA in lymphocytes of HCV-infected individuals (Laskus et al., 2000; Afonso et al., 1999) and the near universal recurrence of infection following liver transplantation (Pessoa and Wright, 1997). Based on their carbohydrate- and glycoprotein-binding properties, and by using a virus binding assay, L-SIGN and DC-SIGN have recently been demonstrated to specifically bind naturally occurring HCV present in the sera of infected individuals (Gardner et al., 2003).

The tissue tropism of HCV is restricted by the envelope glycoproteins E1 and E2 (E1/E2) (McKeating et al., 2004; Lavillette et al., 2004). E1 has homologies to the Class II fusion proteins of other flaviviruses and alphaviruses (Garry and Dash, 2003). E2 is a receptor-binding subunit with affinity for CD81 (Pileri et al., 1998), which serves as an entry coreceptor for HCV (McKeating et al., 2004; Cormier et al., 2004a), and scavenger receptor class B type 1 (SR-B1) (Scarselli et al., 2002), another molecule implicated in HCV entry (Lavillette et al., 2004; Bartosch et al., 2003a; Voisset et al., 2005). It has been demonstrated that L-SIGN also binds HCV E2 and mediates trans-infection of liver cells by HCVpp (Gardner et al., 2003; Pohlmann et al., 2003; Lozach et al., 2003, 2004; Cormier et al., 2004b. A model has been proposed whereby L-SIGN may concentrate HCV in the liver, enable blood-borne virus to cross the endothelial barrier, and facilitate infection of neighboring hepatocytes (Cormier et al., 2004b)

It has also been demonstrated that binding of L-SIGN and DC-SIGN to E2 is blocked by specific inhibitors, including mannan, calcium chelators, and antibodies to the lectin domain of the SIGN molecules (Gardner et al., 2003). The interaction between the SIGN molecules and E2 has further been demonstrated in studies limited to recombinant forms of the HCV glycoproteins (Lozach et al., 2003; Pohlmann et al., 2003). Thus, L-SIGN represents a liver-specific receptor for HCV. L-SIGN and DC-SIGN are also able to mediate internalization of virus particles, as required for cellular entry and infection by HCV but not by HIV-1.

The demonstration that cell surface-expressed L-SIGN mediates binding of HCV virions via the viral envelope glycoprotein, E2 (Gardner et al., 2003), together with the expression of L-SIGN in the liver and lymph nodes, suggests that L-SIGN, by virtue of capturing virus and mediating infection of the susceptible cells, may play critical roles in tissue tropism and viral pathogenesis, including resistance to HCV infection, HCV disease progression and response to antiviral treatments. The expression of L-SIGN on the endothelium of the liver sinusoids and lymph nodes also suggests that it plays an important role in immune surveillance, and therefore represents a potential target for therapy of immunologic diseases. U.S. patent application Ser. Nos. 10/184,150 and 10/328,997 describe the use of DC-SIGN and L-SIGN in compositions and methods for inhibiting HCV infection. The entire contents of these applications are incorporated herein by reference.

The L-SIGN gene is polymorphic in Exon 4, as manifest by a variable number of tandem 69-base pair repeats (Soilleux et al., 2000; Bashirova et al., 2001; Liu et al., 2003; Feinberg et al., 2004) encoding the 23-amino acid repeats. Each of these 23-amino acid repeats comprises a hydrophobic heptad motif characteristic of α-helical coiled coils and related oligomeric structures (Feinberg et al., 2004; Mitchell et al., 2001). The polymorphic L-SIGN alleles can be distinguished using sequence-specific PCR primers and gel electrophoresis.

The nucleic acid sequences of the 69-base pair repeats of Exon 4, as deposited in the Genbank database (AF209481, allele 7), are depicted in FIG. 1a. The deduced amino acid sequences of the encoded 23 amino acid repeats (Genbank database AY042234) are depicted in FIG. 1b. A consensus sequence for the amino acid repeats, as reported by Soilleux et al. (2000), is shown in FIG. 1c.

To assess whether polymorphisms in the L-SIGN repeat region could affect individual HIV-1 susceptibility and subsequent disease progression, Liu et al. (2003) recently analyzed L-SIGN repeat polymorphisms in diverse cohorts of exposed seronegative, HIV-1-infected, long-term nonprogressor and HIV-1-negative Caucasian individuals. They identified 7 alleles in the L-SIGN repeat region based on numbers of tandem repeats (ranging from 3 to 9), and these alleles were combined into 15 different genotypes. The allele frequency in the studied Caucasian populations varied enormously, ranging from 0.1% for allele 3 (3 tandem repeats) to 53.7% for allele 7 (7 tandem repeats) (Liu et al., 2003). Bashirova et al. (2001) reported similar allele frequencies in Caucasians, with the most common L-SIGN-7 allele (encoding 7 repeat segments) comprising just over 50% of all alleles, and other alleles encoding from 3 to 9 tandem repeats (L-SIGN-3 to L-SIGN-9) varying in frequency from 0.3% (L-SIGN-3) to 29% (L-SIGN-5). An independent study on a Japanese population identified only 5 alleles in the L-SIGN repeat region with the number of tandem repeats ranging from 5 to 9 (Kobayashi et al., 2002). However, little is known about the expression and function of these L-SIGN isoforms.

By correlating the frequency of genotypes with the risk of acquiring HIV-1 infection in each category of the studied cohorts, Liu et al. (2003) discovered that homozygous L-SIGN alleles with 7 repeats (7/7) are associated with increased susceptibility to HIV-1 infection, whereas the heterozygous 7/5 or homozygous 5/5 genotype correlated with a decreased incidence of HIV-1 infection. They therefore concluded that the L-SIGN allele containing five Exon 4 repeats may act as a dominant resistance factor whereas the 7-repeat allele may act as a recessive susceptibility factor for HIV-1.

Kobayashi et al. (2002) also analyzed the effect of single nucleotide polymorphisms (SNPs) in L-SIGN on HIV-1 pathogenesis in a cohort of 110 HIV-infected Japanese patients. They reported that the presence of particular SNP alleles correlated with the lowest CD4+ cell counts in the patients tested.

By analogy with the effect of L-SIGN polymorphisms on HIV-1 infectivity and pathogenesis, structural variants of L-SIGN resulting from allelic polymorphisms may bind with different avidities to HCV and other L-SIGN-binding pathogens, including simian immunodeficiency virus, dengue virus, Ebola virus, cytomegalovirus, Marburg virus, SARS coronavirus, Sindbis virus and Plasmodium sporozoites. This differential binding may thereby affect resistance or susceptibility to infection by these pathogens.

The present invention provides a molecular mechanism whereby genetic polymorphisms in L-SIGN and DC-SIGN could influence the establishment and progression of diseases mediated by HCV and other pathogens recognized by L-SIGN and DC-SIGN, and in particular, could afford protection against pathogen infection and disease progression. The invention also describes the novel application of L-SIGN polymorphisms as a tool for diagnosing, preventing and treating pathogen-related diseases.

### SUMMARY OF THE INVENTION

This invention provides a method for determining whether an agent preferentially binds to at least one allelic variant of L-SIGN, comprising: (a) separately contacting an agent with one of (i) at least two allelic variants of L-SIGN, (ii) at least two cell lines expressing allelic variants of L-SIGN, and (iii) plasma membrane fractions from extracts of the at least two cell lines, under conditions suitable for binding of the agent; and (b) comparing the relative binding of the agent to the allelic variants, or to the cells or membrane fractions expressing allelic variants with which the agent is contacted, wherein a difference in relative binding indicates that the agent preferentially binds to at least one allelic variant of L-SIGN.

This invention also provides a method for determining whether an agent preferentially binds to a first allelic variant of L-SIGN, comprising: (a) separately contacting an agent with (1) both a first allelic variant and a second allelic variant of L-SIGN known to bind to the agent, and with (2) only the second variant, under conditions suitable for binding of the agent to both the first and the second allelic variant; and (b) comparing the extent of binding of the agent to the second variant in the absence versus in the presence of the first variant, wherein a smaller extent of binding of the agent to the second variant in the presence of the first variant indicates that the first variant inhibits the binding of the agent to the second variant, thereby identifying the first allelic variant as one to which the agent preferentially binds.

This invention further provides a method for determining whether a first agent preferentially binds to an allelic variant of L-SIGN, comprising: (a) separately contacting one of (i) an allelic variant of L-SIGN, (ii) cells expressing the allelic variant, and (iii) plasma membrane fractions from such cells with (1) both a first agent and a second agent known to bind to the allelic variant, and (2) only the second agent, under conditions suitable for binding of the allelic variant to both the first and the second agent; and (b) comparing the extent of binding of the allelic variant, or cells or membrane fractions expressing this variant, to the second agent in the absence versus in the presence of the first agent, wherein a smaller extent of binding of the allelic variant to the second agent in the presence of the first agent indicates that the first agent inhibits the binding of the allelic variant to the second agent, so as to thereby determine whether the first agent preferentially binds to the allelic variant of L-SIGN.

This invention still further provides a method for screening a plurality of agents, not known to bind to any allelic variant of L-SIGN, to identify an agent that preferentially binds to at least one such allelic variant of L-SIGN, which method comprises: (a) separately contacting one of (i) an allelic variant of L-SIGN, (ii) cells expressing the allelic variant, and (iii) plasma membrane fractions from such cells, with (1) both a plurality of agents and a binding agent known to bind to the allelic variant, and (2) only such binding agent, under conditions suitable for binding of both the binding agent and the plurality of agents to the allelic variant; (b) comparing the extent of binding to the allelic variant, or cells or membrane fractions expressing this variant, of the binding agent in the presence versus the absence of the plurality of agents; and (c) if the extent of binding of the binding agent to the allelic variant is reduced in the presence of the plurality of agents, separately assessing the strength of the binding of each agent present in the plurality of agents to the allelic variant, so as to thereby identify any agent present in the plurality of agents that preferentially binds to the allelic variant.

This invention additionally provides a method for identifying a monoclonal antibody that specifically binds to an allelic variant of L-SIGN, comprising: (a) administering to a subject an allelic L-SIGN variant protein or an expression vector comprising a nucleic acid which encodes this allelic L-SIGN variant protein; (b) harvesting antibody-producing lymphatic cells from the subject; (c) generating hybridomas by fusing single antibody-producing cells obtained in the harvesting step with myeloma cells; and (d) screening hybridoma supernatants from these hybridomas by any of the methods described herein to identify a monoclonal antibody that specifically binds to the allelic variant of L-SIGN.

The present invention also provides an agent that preferentially binds at least one allelic variant of L-SIGN.

This invention further provides a composition comprising the agent described herein and a carrier.

In addition, this invention provides a method for treating a subject afflicted with a pathogen-related disorder, susceptibility to which is associated with at least one polymorphism in L-SIGN, which method comprises administering to the subject an agent, wherein this agent is (1) determined to preferentially bind to at least one allelic variant of L-SIGN using a method comprising: (a) separately contacting an agent with one of (i) at least two allelic variants of L-SIGN, (ii) at least two cell lines expressing allelic variants of L-SIGN, and (iii) plasma membrane fractions from extracts of the at least two cell lines, under conditions suitable for binding of the agent; and (b) comparing the relative binding of the agent to the allelic variants, or to the cells or membrane fractions expressing allelic variants, wherein a difference in relative binding indicates that the agent preferentially binds to at least one allelic variant of L-SIGN; and (2) administered to the subject in a therapeutically effective amount to treat the subject.

This invention also provides a method for treating a subject afflicted with a pathogen-related disorder, susceptibility to which is associated with at least one polymorphism in L-SIGN, which method comprises administering to the subject an agent, wherein this agent is (1) determined to preferentially bind to a first allelic variant of L-SIGN using a method comprising: (a) separately contacting an agent with (i) both a first allelic variant and a second allelic variant of L-SIGN known to bind to the agent, and with (ii) only the second variant, under conditions suitable for binding of the agent to both the first and the second allelic variant; and (b) comparing the extent of binding of the agent to the second variant in the absence versus in the presence of the first variant, wherein a smaller extent of binding of the agent to the second variant in the presence of the first variant indicates that the first variant inhibits the binding of the agent to the second variant, thereby identifying the first allelic variant as one to which the agent preferentially binds; and wherein the agent is (2) administered to the subject in a therapeutically effective amount to treat the subject.

This invention further provides a method for treating a subject afflicted with a pathogen-related disorder, susceptibility to which is associated with at least one polymorphism in L-SIGN, which method comprises administering to the subject a first agent, wherein this first agent is determined to preferentially bind to an allelic variant of L-SIGN using a method comprising: (a) separately contacting one of (i) an allelic variant of L-SIGN, (ii) cells expressing the allelic variant, and (iii) plasma membrane fractions from such cells with (1) both a first agent and a second agent known to bind to the allelic variant, and (2) only the second agent, under conditions suitable for binding of the allelic variant to both the first and the second agent; and (b) comparing the extent of binding of the allelic variant, or cells or membrane fractions expressing the variant, to the second agent in the absence versus in the presence of the first agent, wherein a smaller extent of binding of the allelic variant to the second agent in the presence of the first agent indicates that the first agent inhibits the binding of the allelic variant to the second agent, so as to thereby determine whether the first agent preferentially binds to the allelic variant of L-SIGN; and the first agent is administered to the subject in a therapeutically effective amount to treat the subject.

This invention still further provides a method for preventing infection of a subject by a pathogen, susceptibility to which infection is associated with at least one polymorphism in L-SIGN, which method comprises administering to the subject an agent, wherein this agent is (1) determined to preferentially bind to at least one allelic variant of L-SIGN using a method comprising: (a) separately contacting an agent with one of (i) at least two allelic variants of L-SIGN, (ii) at least two cell lines expressing allelic variants of L-SIGN, and (iii) plasma membrane fractions from extracts of the at least two cell lines, under conditions suitable for binding of the agent; and (b) comparing the relative binding of the agent to the allelic variants, or to the cells or membrane fractions expressing allelic variants, wherein a difference in relative binding indicates that the agent preferentially binds to at least one allelic variant of L-SIGN; and wherein the agent is (2) administered to the subject in a prophylactically effective amount to prevent infection by the pathogen.

This invention also provides a method for preventing infection of a subject by a pathogen, susceptibility to which infection is associated with at least one polymorphism in L-SIGN, which method comprises administering to the subject an agent, wherein this agent is (1) determined to preferentially bind to a first allelic variant of L-SIGN using a method comprising: (a) separately contacting an agent with (i) both a first allelic variant and a second allelic variant of L-SIGN known to bind to the agent, and with (ii) only the second variant, under conditions suitable for binding of the agent to both the first and the second allelic variant; and (b) comparing the extent of binding of the agent to the second variant in the absence versus in the presence of the first variant, wherein a smaller extent of binding of the agent to the second variant in the presence of the first variant indicates that the first variant inhibits the binding of the agent to the second variant, thereby identifying the first allelic variant as one to which the agent preferentially binds; and wherein the agent is (2) administered to the subject in a prophylactically effective amount to prevent infection by the pathogen.

This invention further provides a method for preventing infection of a subject by a pathogen, susceptibility to which infection is associated with at least one polymorphism in L-SIGN, which method comprises administering to the subject a first agent, wherein this first agent is determined to preferentially bind to an allelic variant of L-SIGN using a method comprising: (a) separately contacting one of (i) an allelic variant of L-SIGN, (ii) cells expressing the allelic variant, and (iii) plasma membrane fractions from such cells with (1) both a first agent and a second agent known to bind to the allelic variant, and (2) only the second agent, under conditions suitable for binding of the allelic variant to both the first and the second agent; and (b) comparing the extent of binding of the allelic variant, or cells or membrane fractions expressing the variant, to the second agent in the absence versus in the presence of the first agent, wherein a smaller extent of binding of the allelic variant to the second agent in the presence of the first agent indicates that the first agent inhibits the binding of the allelic variant to the second agent, so as to thereby determine whether the first agent preferentially binds to the allelic variant of L-SIGN; and wherein the first agent is administered to the subject in a prophylactically effective amount to prevent infection by the pathogen.

This invention still further provides a method for inhibiting in a subject the onset of a pathogen-related disorder, susceptibility to which is associated with at least one polymorphism in L-SIGN, which method comprises administering to the subject an agent, wherein this agent is (1) determined to preferentially bind to at least one allelic variant of L-SIGN using a method comprising: (a) separately contacting an agent with one of (i) at least two allelic variants of L-SIGN, (ii) at least two cell lines expressing allelic variants of L-SIGN, and (iii) plasma membrane fractions from extracts of the at least two cell lines, under conditions suitable for binding of the agent; and (b) comparing the relative binding of the agent to the allelic variants, or to the cells or membrane fractions expressing allelic variants, wherein a difference in relative binding indicates that the agent preferentially binds to at at least one allelic variant of L-SIGN; and wherein the agent is (2) administered in a prophylactically effective amount to have a prophylactic effect in the subject.

This invention additionally provides a method for inhibiting in a subject the onset of a pathogen-related disorder, susceptibility to which is associated with at least one polymorphism in L-SIGN, which method comprises administering to the subject a prophylactically effective amount of an agent, wherein this agent is (1) determined to preferentially bind to a first allelic variant of L-SIGN using a method comprising: (a) separately contacting an agent with (i) both a first allelic variant and a second allelic variant of L-SIGN known to bind to the agent, and with (ii) only the second variant, under conditions suitable for binding of the agent to both the first and the second allelic variant; and (b) comparing the extent of binding of the agent to the second variant in the absence versus in the presence of the first variant, wherein a smaller extent of binding of the agent to the second variant in the presence of the first variant indicates that the first variant inhibits the binding of the agent to the second variant, thereby identifying the first allelic variant as one to which the agent preferentially binds; and wherein the agent is (2) administered to the subject in a prophylactically effective amount to have a prophylactic effect in the subject.

This invention also provides a method for inhibiting in a subject the onset of a pathogen-related disorder, susceptibility to which is associated with at least one polymorphism in L-SIGN, which method comprises administering to the subject a prophylactically effective amount of a first agent, wherein this first agent is determined to preferentially bind to an allelic variant of L-SIGN using a method comprising: (a) separately contacting one of (i) an allelic variant of L-SIGN, (ii) cells expressing the allelic variant, and (iii) plasma membrane fractions from the cells with (1) both a first agent and a second agent known to bind to the allelic variant, and (2) only the second agent, under conditions suitable for binding of the allelic variant to both the first and the second agent; and (b) comparing the extent of binding of the allelic variant, or cells or membrane fractions expressing the variant, to the second agent in the absence versus in the presence of the first agent, wherein a smaller extent of binding of the allelic variant to the second agent in the presence of the first agent indicates that the first agent inhibits the binding of the allelic variant to the second agent, so as to thereby determine whether the first agent preferentially binds to the allelic variant of L-SIGN; and the first agent is administered in a prophylactically effective amount to have a prophylactic effect in the subject.

This invention further provides a method for preventing infection of a subject by a pathogen, which infection is prevented by immunizing the subject, comprising: (a) administering to the subject one of (i) an allelic L-SIGN variant protein substantially identical to an L-SIGN variant associated with membranes of cells of the subject, and (ii) an expression vector comprising a nucleic acid that encodes the allelic L-SIGN variant protein; so as to thereby (b) elicit in the subject the production of L-SIGN-specific antibodies which inhibit binding of the pathogen to the allelic L-SIGN variant associated with membranes of the subject's cells, wherein these antibodies are not harmful to the subject.

This invention still further provides a method for inhibiting in a subject the onset of a pathogen-related disorder, the inhibition of which is effected by immunizing the subject, which method comprises: (a) administering to the subject one of (i) an allelic L-SIGN variant protein substantially identical to an L-SIGN variant associated with membranes of the subject's cells, and (ii) an expression vector comprising a nucleic acid that encodes the allelic L-SIGN variant protein; so as to thereby (b) elicit in the subject the production of L-SIGN-specific antibodies which inhibit binding of a pathogen to the allelic L-SIGN variant associated with membranes of the subject's cells, wherein these antibodies are not harmful to the subject.

This invention also provides a method for predicting resistance of a subject to infection by a pathogen by determining the status of L-SIGN Exon 4 repeat polymorphisms in the subject and correlating that status to a degree of resistance of the subject to the pathogen, which method comprises: (a) extracting genomic DNA from cells of the subject; (b) amplifying the DNA by a polymerase chain reaction (PCR) using primers that are specific for Exon 4 of L-SIGN; (c) identifying the L-SIGN alleles present by determining the size of the amplified DNA, wherein the size of the amplified DNA is proportional to the number of Exon 4 repeats in the allele; and (d) correlating the identity of the L-SIGN alleles in the subject with allelic combinations known to be associated with resistance to infection by the pathogen.

Additionally, the present invention provides a method for predicting susceptibility of a subject to infection by a pathogen by determining the status of L-SIGN Exon 4 repeat polymorphisms in the subject and correlating that status to a degree of susceptibility of the subject to the pathogen, which method comprises: (a) extracting genomic DNA from cells of the subject; (b) amplifying the DNA by a polymerase chain reaction (PCR) using primers that are specific for Exon 4 of L-SIGN; (c) identifying the L-SIGN alleles present by determining the size of the amplified DNA, wherein the size of the amplified DNA is proportional to the number of Exon 4 repeats in the allele; and (d) correlating the identity of the L-SIGN alleles in the subject with allelic combinations known to be associated with susceptibility to infection by the pathogen.

This invention also provides a method for predicting resistance of a subject to infection by a pathogen by identifying single nucleotide L-SIGN polymorphisms in the subject and correlating the presence of these single nucleotide polymorphisms (SNPs) to the resistance of the subject to the pathogen, which method comprises: (a) extracting genomic DNA from cells of a subject; (b) amplifying the genomic DNA by a polymerase chain reaction (PCR) using primers that are specific for regions within L-SIGN alleles; (c) screening the amplified DNA to detect SNPs; and (d) correlating the identity of alleles containing SNPs so detected with allelic combinations known to be associated with resistance to infection by the pathogen.

This invention further provides a method for predicting the susceptibility of a subject to infection by a pathogen by identifying single nucleotide L-SIGN polymorphisms in the subject and correlating the presence of these single nucleotide polymorphisms (SNPs) to the susceptibility of the subject to the pathogen, which method comprises: (a) extracting genomic DNA from cells of a subject; (b) amplifying the genomic DNA by a polymerase chain reaction (PCR) using primers that are specific for regions within L-SIGN alleles; (c) screening the amplified DNA to detect SNPs; and (d) correlating the identity of alleles containing SNPs so detected with allelic combinations known to be associated with susceptibility to infection by the pathogen.

This invention still further provides a method for predicting resistance of a subject to infection by a pathogen by identifying an L-SIGN polymorphism in the subject other than a single nucleotide polymorphism (SNP) or an Exon 4 repeat polymorphism, and correlating the presence of this L-SIGN polymorphism to the resistance of the subject to the pathogen, which method comprises: (a) extracting genomic DNA from cells of a subject; (b) amplifying the genomic DNA by a polymerase chain reaction (PCR) using primers that are specific for regions of L-SIGN; (c) sequencing the amplified DNA and comparing the sequence to known sequences of L-SIGN alleles to detect any polymorphisms present; and (d) correlating the identity of alleles containing a detected polymorphism, wherein this polymorphism is not a SNP or an Exon 4 repeat polymorphism, with allelic combinations known to be associated with resistance to infection by the pathogen.

This invention additionally provides a method for predicting susceptibility of a subject to infection by a pathogen by identifying an L-SIGN polymorphism in the subject other than a single nucleotide polymorphism (SNP) or an Exon 4 repeat polymorphism, and correlating the presence of this L-SIGN polymorphism to the susceptibility of the subject to the pathogen, which method comprises: (a) extracting genomic DNA from cells of a subject; (b) amplifying the genomic DNA by a polymerase chain reaction (PCR) using primers that are specific for regions of L-SIGN; (c) sequencing the amplified DNA and comparing the sequence to known sequences of L-SIGN alleles to detect any polymorphisms present; and (d) correlating the identity of alleles containing a detected polymorphism, wherein this polymorphism is not a SNP or an Exon 4 repeat polymorphism, with allelic combinations known to be associated with susceptibility to infection by the pathogen.

The present invention also provides an article of manufacture comprising a packaging material containing therein an agent as described herein and a label providing instructions for using the agent to treat a subject afflicted with a pathogen-associated disorder.

This invention further provides an article of manufacture comprising a packaging material containing therein an agent as described herein and a label providing instructions for using the agent to prevent infection of a subject by a pathogen.

This invention also provides an article of manufacture comprising a packaging material containing therein an agent as described herein and a label providing instructions for using the agent to inhibit the onset of a pathogen-associated disorder in a subject.

This invention further provides an article of manufacture comprising a packaging material containing therein one of (i) an allelic L-SIGN protein variant substantially identical to an L-SIGN variant associated with membranes of cells of a subject, and (ii) an expression vector comprising a nucleic acid that encodes this allelic L-SIGN protein variant, and a label providing instructions for using the L-SIGN variant protein or expression vector to prevent infection of the subject by a pathogen, which infection is prevented by using the L-SIGN protein variant as an immunogen to immunize the subject.

This invention still further provides an article of manufacture comprising a packaging material containing therein one of (i) an allelic L-SIGN protein variant substantially identical to an L-SIGN variant associated with membranes of cells of a subject, and (ii) an expression vector comprising a nucleic acid that encodes this allelic L-SIGN protein variant, and a label providing instructions for using the L-SIGN variant protein or expression vector to inhibit in a subject the onset of a pathogen-related disorder, the inhibition of which is effected by using the L-SIGN protein variant as an immunogen to immunize the subject.

### BRIEF DESCRIPTION OF THE FIGURES

FIG. 1. Nucleotide and encoded amino acid sequences of L-SIGN Exon 4 repeats. (a) Nucleotide alignment of known L-SIGN repeat sequences, based on the DNA sequence deposited in the Genbank database (AF209481). Three of the 7 repeats (R3, R4 and R5) have identical nucleotide sequences; hence, there are 5 known unique repeat sequences. Others may exist in nature as there are known to be up to 9 repeat alleles of L-SIGN. The nucleotide sequences shown for R1, R2, R6 and R7 are assigned SEQ ID NOs. 1, 2, 4 and 5, respectively. The identical sequence of R3, R4 and R5 is designated SEQ ID NO:3. (b) Alignment of the deduced amino acid sequences of the L-SIGN repeats (Genbank database AY042234). The amino acid sequences shown for R1, R2, R6 and R7 are designated SEQ ID NOs. 6, 7, 9 and 10, respectively. The identical sequence of R3, R4 and R5 is designated SEQ ID NO:8. R1-R7 represent L-SIGN Exon 4 repeat regions 1-7, respectively. Nucleotide and amino acid differences among the repeat sequences are shaded. (c) Consensus sequence (SEQ ID NO:11) for the L-SIGN amino acid repeats (Soilleux et al., 2000). The first residue of the consensus amino acid repeat as defined by Soilleux et al. (2000) corresponds to residue 9 of the repeats depicted in (b).

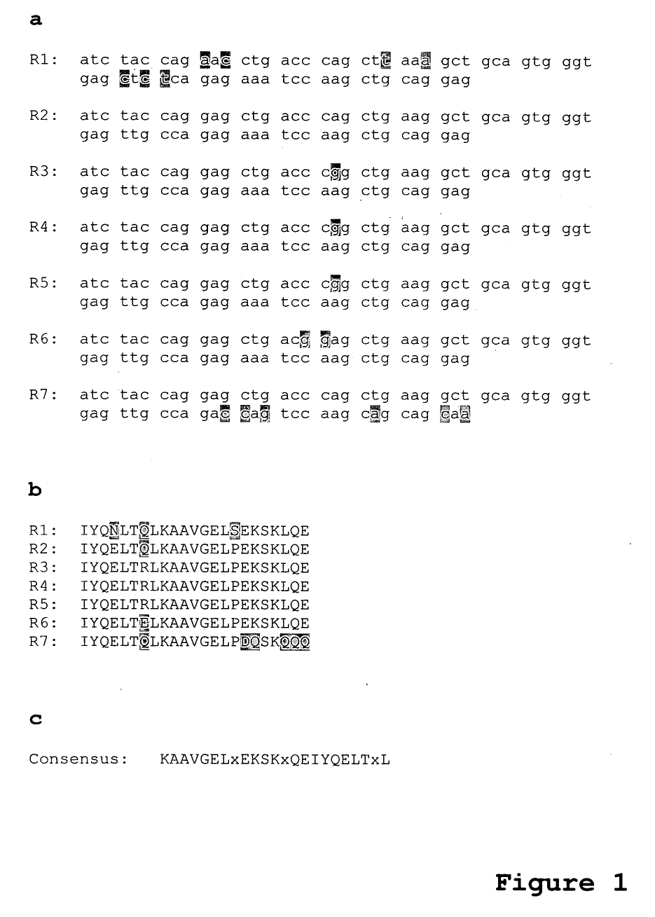

FIG. 2. Retroviral vectors for generating pseudotypes. (a) MMLV-based pFB vector (Stratagene) encodes a packageable genome that expresses lacZ and neoR from a single mRNA wherein translation of neoR is driven by an IRES. pVPack expresses Gag and Pol, which make up the nucleocapsid. pVPack-VSV G encodes the VSV envelope glycoprotein G, used as a positive control of viral entry because it efficiently mediates entry into most cells. pcDNA3.1 expresses HCV envelope glycoproteins. E1* comprises nucleotides 511-1149, E2* comprises nucleotides 1111-2238, and E1*-E2* comprises nucleotides 511-2238 of HCV. (b) Similarly, HIV-1-based pLenti6 vector (Invitrogen) encodes a packageable, self-inactivating, bicistronic genome that encodes lacZ and blast. pLP1 expresses Gag and Pol, which make up the nucleocapsid. pLP2 expresses Rev and pVPack-VSV G encodes the VSV envelope glycoprotein G. pcDNA3.1 expresses HCV envelope glycoproteins. LTR=long terminal repeat; CMV=Cytomegalovirus promoter; RSV=Rous Sarcoma Virus promoter; SV40=Simian virus 40 early promoter; IRES=internal ribosomal entry site, pA=poly A sequence; RRE=Rev responsive element, ⋄=packaging signal; SD=splice donor; SA=splice acceptor; hisD=histidinol resistance, puro=puromycin resistance, blast=blasticidin resistance; neoR=neomycin resistance; lacZ=β-galactosidase gene.

FIG. 3. Hybridoma supernatants show reactivity with E2. (a) 293T cells or 293T transiently expressing E1/E2 were solubilized with M-PER® lysis buffer (Pierce). Microtiter plates were coated with cell lysates or soluble E2 proteins and subjected to ELISA as described in the Methods. (b) Flow cytometric analysis of HeLa-E1*-E2* cells incubated with hybridoma supernatants. (c) Western blot analysis. 293T transiently expressing E1/E2 were lysed in 1% NP40 buffer and proteins separated by SDS-PAGE, then transferred to nitrocellulose membranes. Membranes were probed with the indicated mAbs and visualized by chemifluorescence (see Methods).

FIG. 4. Domain structures of the proteins encoded by L-SIGN repeat-region isoforms. The 23-amino-acid tandem repeats within the neck region are numbered according to those present in L-SIGN-7 (Accession #NP_{—}055072). The first tandem repeat begins at Ile-89. Each allele also encodes a partial (15 amino acid) repeat segment carboxy-terminal to repeat 7 (not shown). Cyto=cytoplasmic domain; TM=transmembrane domain.

FIG. 5. Amino acid sequences of L-SIGN isoforms. The deduced amino acid sequences of L-SIGN isoforms containing 3, 4, 5, 7, and 9 tandem repeats are indicated. Each isoform encodes an amino-terminal cytoplasmic domain followed by a 22 amino acid transmembrane domain. The extracellular region contains 3-9 tandem repeats of a 23 amino acid sequence plus a 15 amino acid partial repeat segment. The carboxy-terminal portion of each protein consists of a carbohydrate-recognition domain that is 135 amino acids in length.

FIG. 6. Cell-surface expression of L-SIGN repeat-region isoforms. HeLa cells were stably transfected to express repeat-region isoforms of L-SIGN and analyzed by flow cytometry using anti-CRD mAb 120604 or anti-repeat-region mAb DC28. In contrast, background levels of binding were observed using an isotype-control antibody and DC-SIGN-specific mAb 120507 (data not shown). MFI=mean fluorescence intensity.

FIG. 7. Binding of soluble E2 and HCVpp to L-SIGN isoforms. Parental HeLa or HeLa-SIGN transfectants were incubated with soluble E2 glycoprotein for 1 h at 4° C. Cells were washed and bound E2 was detected by flow cytometry using mouse anti-E2 mAb followed by FITC-conjugated goat anti-mouse antibody. The data reflect the difference in the percentage of positively-stained cells observed in the presence and absence of E2. The background binding to parental HeLa cells analyzed in parallel was 3.8%. For measurement of HCVpp binding, cells were incubated with HCVpp-containing supernatants for 1 h at 37° C., washed and lysed. The p24 content of the lysates was measured by ELISA. The values reflect the net binding to HeLa-SIGN transfectants after subtraction of background binding to parental HeLa cells analyzed in parallel. Data from one of three representative experiments are shown.

FIG. 8. Allelic variation in L-SIGN-mediated trans-infection of Hep3B cells by HCVpp. HCVpp were incubated with parental HeLa or HeLa-SIGN transfectants stably expressing the indicated polymorphic forms of L-SIGN. Cells were washed and then co-cultured with Hep3B cells for 48 h. Cultures were lysed and luciferase activity (relative light units, RLU) was measured. Data represent the mean and standard deviations of three independent experiments performed in the presence and absence of isotype-control mouse IgG. Data represent the net RLU values after subtraction of RLU observed for parental HeLa. When tested against the hypothesis μ=0, the levels of trans-infection were statistically significant for L-SIGN-4, -5, -7 and -9 but not L-SIGN-3 in 2-sided t-tests, and the P values are indicated. Compared to L-SIGN-7, significantly lower levels of trans-infection were observed for L-SIGN-3 (P=0.011), L-SIGN-4 (P=0.033) and L-SIGN-5 (P=0.049) according to 2-sided t-tests, while the data for L-SIGN-9 trended towards significance (P=0.049 and P=0.099 in 1- and 2-sided tests, respectively).

FIG. 9. Inhibition of trans-infection with agents to the CRD of L-SIGN. HeLa or HeLa-SIGN transfectants stably expressing the indicated polymorphic forms of L-SIGN were incubated with mannan (20 μg/ml), anti-CRD mAbs (120604 and 120612, 10 μg/ml), or IgG2a isotype-control IgG (10 μg/ml) prior to addition to HCVpp. Cells were incubated an additional 2 h, washed and combined with Hep3B cells. Cultures were maintained for 48 h prior to lysis and measurement of luciferase activity. The data represent the percent of trans-infection relative to that observed in the absence of inhibitor. The values were calculated as (net RLU in the presence of inhibitor)/(net RLU in the absence of inhibitor)×100, where net RLU represents the difference in RLU values observed between transfected and parental HeLa cells. Negative values are omitted for clarity. The data represent the average of 3 independent measurements. The MEAN value represents the average of the inhibition values observed for the different L-SIGN alleles. N/A=not applicable. L-SIGN-3 did not mediate trans-infection in the presence or absence of inhibitors.

### DETAILED DESCRIPTION OF THE INVENTION

### Definitions

As used in this application, except as otherwise expressly provided herein, each of the following terms shall have the meaning set forth below.

“Administering” shall mean delivering in a manner which is effected or performed using any of the various methods and delivery systems known to those skilled in the art. Administering can be performed, for example, topically, intravenously, pericardially, orally, parenterally, via implant, trans-mucosally, transdermally, intradermally, intramuscularly, subcutaneously, intraperitoneally, intrathecally, intralymphatically, intra-lesionally, or epidurally. An agent or composition may also be administered in an aerosol, such as for pulmonary and/or intranasal delivery. Administering can also be performed, for example, once, a plurality of times, and/or over one or more extended periods.

An “antibody” shall mean an immunoglobulin molecule, comprising two heavy chains and two light chains and monovalent and divalent fragments thereof, which recognizes an antigen. The immunoglobulin molecule may derive from any of the commonly known classes, including but not limited to IgA, secretory IgA, IgG and IgM. IgG subclasses are also well known to those in the art and include but are not limited to human IgG1, IgG2, IgG3 and IgG4. The term “antibody” includes, by way of example, both naturally occurring and non-naturally occurring antibodies, polyclonal and monoclonal antibodies, and monovalent and divalent fragments thereof. Furthermore, “antibody” includes chimeric, humanized and human antibodies, wholly synthetic antibodies, single-chain antibodies, and antigen-binding fragments thereof. Optionally, an antibody can be labeled with a detectable marker, including, for example, a radioactive or fluorescent marker.

A “human” antibody shall mean an antibody wherein all of the amino acids correspond to amino acids in human immunoglobulin molecules.

A “humanized” antibody shall mean an antibody wherein some, most or all of the amino acids outside the complementarity determining regions (CDRs) are replaced with corresponding amino acids derived from human immunoglobulin molecules.

A “monoclonal antibody,” also designated a mAb, is used to describe antibody molecules whose primary sequences are essentially identical and which exhibit the same antigenic specificity. Monoclonal antibodies may be produced by hybridoma, recombinant, transgenic or other techniques known to one skilled in the art.

A “disease” or “disorder” is an abnormal physical or mental condition that occurs in a subject which impairs proper functioning of the subject or at least one of its parts.

A “disease-associated allelic variant” of L-SIGN refers to an L-SIGN allelic subtype, the presence of which in a subject correlates with increased susceptibility of the subject to a disease. A “non-disease-associated allelic variant” of L-SIGN refers to an L-SIGN allelic subtype, the presence of which in a subject does not correlate with increased susceptibility to a disease.

“Exon 4 repeat polymorphisms” in the L-SIGN gene shall mean the existence of different alleles encoding L-SIGN, wherein these alleles arise from a variable number (three to nine) of tandem repeats of a 69 nucleotide-long sequence within Exon 4 of the L-SIGN gene.

“Harmful to a subject” shall mean of a kind likely to be damaging or deleterious to the health of the subject.

“HCV” shall mean the hepatitis C virus without limitation to strain, subtype or genotype, such as are disclosed in U.S. Pat. Nos. 6,572,864 and 5,882,852. HCV includes but is not limited to extracellular virus particles and the forms of HCV associated with and/or found in HCV-infected cells.

“Intercellular adhesion molecule-3 (ICAM-3)-grabbing nonintegrin molecules related” to L-SIGN include, but are not limited to, DC-SIGN.

In general, “pathogen infection” shall mean the establishment of a growing population of a pathogen in a suitable host cell following binding of the pathogen to the host cell membrane. For viral pathogens in particular, such as HCV, “infection” shall mean the introduction of viral genetic information into a host cell, following the fusion of the host cell membrane with virions or a viral envelope glycoprotein⁺ cell. In one embodiment, the host cell is a bodily cell from a subject, such as from a human subject. Infection is usually but not necessarily accompanied by the induction of disease symptoms in a subject. As used herein, “inhibiting pathogen infection” shall mean reducing the extent of the establishment of a pathogen in a host cell as compared to the extent of the establishment that would occur without, for example, an inhibiting agent. In a preferred embodiment, “inhibiting” means that the extent of the establishment is reduced 100%. For viral pathogens such as HCV, “inhibiting infection” shall mean reducing the amount of viral genetic information introduced into a host cell as compared to the amount that would be introduced without, for example, an inhibiting agent.

A “pathogen-related” or “pathogen-associated” disorder shall mean a disorder that occurs in a subject concurrently with or subsequent to infection by a pathogen, and is therefore likely to be caused, directly or indirectly, by the pathogen.

“Pharmaceutically acceptable carriers” are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Preferentially binding” means binding to a particular entity with greater avidity than to other related entities.

A “prophylactically effective amount” is any amount of an agent which, when administered to a subject prone to suffer from a disorder, inhibits the onset of the disorder. “Inhibiting” the onset of a disorder means either lessening the likelihood of the disorder's onset, or preventing the onset of the disorder entirely. In the preferred embodiment, inhibiting the onset of a disorder means preventing its onset entirely.

“Subject” means any animal, such as a mammal or a bird, including, without limitation, a human, a non-human primate, a cow, a horse, a sheep, a pig, a dog, a cat, a rabbit, a rodent such as a mouse, rat or guinea pig, a turkey or a chicken. In a preferred embodiment, the subject is a human being.

A “therapeutically effective amount” is any amount of an agent which, when administered to a subject afflicted with a disorder against which the agent is effective, causes the subject to be treated.

“Treating” a subject afflicted with a disorder shall mean causing the subject to experience a reduction, remission or regression of the disorder and/or its symptoms. In one embodiment, recurrence of the disorder and/or its symptoms is prevented. In the preferred embodiment, the subject is cured of the disorder and/or its symptoms.

“Suitable conditions” shall have a meaning dependent on the context in which this term is used. Generally, it means conditions that permit an agent, capable of doing something, to do that intended thing. When used in connection with contacting an agent to a receptor molecule, this term means conditions that permit an agent, capable of doing so, to bind to the receptor molecule. In one embodiment, the term “suitable conditions” as used herein means physiological conditions.

### Embodiments of the Invention

Seven L-SIGN alleles containing 3 to 9 tandem repeats in the repeat region of Exon 4 have been found in human populations, and these alleles were combined into 15 different genotypes (Liu et al., 2003). Different genotypes, comprising particular combinations of L-SIGN alleles, are associated with susceptibility or resistance to infection with a variety of disease-causing pathogens. The present invention is based on novel methods for using detectable L-SIGN polymorphisms in a subject for predicting the susceptibility of the subject to infection by HCV and other pathogens, and for preventing and/or treating pathogen-related diseases. Notably, it has been demonstrated, inter alia, that L-SIGN repeat-region isoforms are efficiently expressed at the surface of mammalian cells, bind HCV envelope glycoprotein E2 and HCV pseudovirus particles (HCVpp) to comparable levels, but mediate trans-infection with decreasing efficiency as the tandem repeats are progressively deleted. In this regard, L-SIGN-3 did not mediate any trans-infection by HCVpp in the experimental system described herein.

Novel agents that bind preferentially to particular L-SIGN allelelic variants, and thus may act to inhibit or prevent binding between certain pathogens and such allelic variants, are identified using screening methods described herein. Specifically, the present invention provides a method for determining whether an agent preferentially binds to at least one allelic variant of L-SIGN, comprising: (a) separately contacting an agent with one of (i) at least two allelic variants of L-SIGN, (ii) at least two cell lines expressing allelic variants of L-SIGN, and (iii) plasma membrane fractions from extracts of the at least two cell lines, under conditions suitable for binding of the agent; and (b) comparing the relative binding of the agent to the allelic variants, or to the cells or membrane fractions expressing allelic variants with which the agent is contacted, wherein a difference in relative binding indicates that the agent preferentially binds to at least one allelic variant of L-SIGN.

This invention also provides a method for determining whether an agent preferentially binds to a first allelic variant of L-SIGN, comprising: (a) separately contacting an agent with (1) both a first allelic variant and a second allelic variant of L-SIGN known to bind to the agent, and with (2) only the second variant, under conditions suitable for binding of the agent to both the first and the second allelic variant; and (b) comparing the extent of binding of the agent to the second variant in the absence versus in the presence of the first variant, wherein a smaller extent of binding of the agent to the second variant in the presence of the first variant indicates that the first variant inhibits the binding of the agent to the second variant, thereby identifying the first allelic variant as one to which the agent preferentially binds.

This invention further provides a method for determining whether a first agent preferentially binds to an allelic variant of L-SIGN, comprising: (a) separately contacting one of (i) an allelic variant of L-SIGN, (ii) cells expressing the allelic variant, and (iii) plasma membrane fractions from such cells with (1) both a first agent and a second agent known to bind to the allelic variant, and (2) only the second agent, under conditions suitable for binding of the allelic variant to both the first and the second agent; and (b) comparing the extent of binding of the allelic variant, or cells or membrane fractions expressing this variant, to the second agent in the absence versus in the presence of the first agent, wherein a smaller extent of binding of the allelic variant to the second agent in the presence of the first agent indicates that the first agent inhibits the binding of the allelic variant to the second agent, so as to thereby determine whether the first agent preferentially binds to the allelic variant of L-SIGN. In one embodiment, the first agent is not previously known to bind to the allelic variant of L-SIGN.

This invention still further provides a method for screening a plurality of agents, not known to bind to any allelic variant of L-SIGN, to identify an agent that preferentially binds to at least one allelic variant of L-SIGN, which method comprises: (a) separately contacting one of (i) an allelic variant of L-SIGN, (ii) cells expressing the allelic variant, and (iii) plasma membrane fractions from such cells, with (1) both a plurality of agents and a binding agent known to bind to the allelic variant, and (2) only such binding agent, under conditions suitable for binding of both the binding agent and the plurality of agents to the allelic variant; (b) comparing the extent of binding to the allelic variant, or cells or membrane fractions expressing this variant, of the binding agent in the presence versus the absence of the plurality of agents; and (c) if the extent of binding of the binding agent to the allelic variant is reduced in the presence of the plurality of agents, separately assessing the strength of the binding of each agent present in the plurality of agents to the allelic variant, so as to thereby identify any agent present in the plurality of agents that preferentially binds to the allelic variant.

Methods for producing monoclonal antibodies are well known in the art (see, e.g., Kohler and Milstein, 1975). This invention additionally provides a method for identifying a monoclonal antibody that specifically binds to an allelic variant of L-SIGN, comprising: (a) administering to a subject an allelic L-SIGN variant protein or an expression vector comprising a nucleic acid which encodes this allelic L-SIGN variant protein; (b) harvesting antibody-producing lymphatic cells from the subject; (c) generating hybridomas by fusing single antibody-producing cells obtained in the harvesting step with myeloma cells; and (d) screening hybridoma supernatants from these hybridomas by any of the methods described herein to identify a monoclonal antibody that specifically binds to the allelic variant of L-SIGN.

Aministration of the L-SIGN variant or encoding nucleic acid may be performed in several ways, including but not limited to intravenous, intramuscular or subcutaneous injection. In one embodiment of the instant method, the expression vector is formulated prior to administration with an adjuvant, for example a liposome, that mediates entry of the vector into cells wherein expression of the L-SIGN variant occurs. In another embodiment, the antibody-producing cells are harvested from the spleen.

Antibodies that specifically bind to a disease-associated allelic variant of L-SIGN may inhibit the binding of pathogens, including HCV, to L-SIGN and thereby block L-SIGN-mediated infection and trans-infection of cells by pathogens. Such antibodies may be used therapeutically to inhibit pathogen infection or to treat subjects afflicted with pathgen-related disorders. They may also be used diagnostically to genetically screen for the presence of disease-associated allelic variants of L-SIGN which correlate with susceptibility of a subject to infection by pathogens that bind to L-SIGN. In one embodiment of the above method, the monoclonal antibody preferentially binds at least one disease-associated allelic variant with at least 2 times greater avidity than it binds at least one non-disease-associated allelic variant. In another embodiment, the monoclonal antibody preferentially binds at least one disease-associated allelic variant with at least 5 times greater avidity than it binds at least one non-disease-associated allelic variant. In a preferred embodiment, the monoclonal antibody preferentially binds at least one disease-associated allelic variant with at least 10 times greater avidity than it binds at least one non-disease-associated allelic variant. In another embodiment, the monoclonal antibody preferentially binds to L-SIGN-7 or L-SIGN-9, and binds less efficiently to one or more of L-SIGN-3, L-SIGN-4 or L-SIGN-5. In a preferred embodiment, the monoclonal antibody preferentially binds to L-SIGN-7.

Antibodies that specifically bind to a non-disease-associated allelic variant of L-SIGN have diagnostic applications, such as, for example, detecting the presence or absence of the non-disease-associated allelic variant, and thereby genetically screening for resistance or susceptibility of a subject to infection by pathogens that bind to L-SIGN. In one embodiment of the above method, the monoclonal antibody preferentially binds at least one non-disease-associated allelic variant with at least 2 times greater avidity than it binds at least one disease-associated allelic variant. In another embodiment, the monoclonal antibody preferentially binds at least one non-disease-associated allelic variant with at least 5 times greater avidity than it binds at least one disease-associated allelic variant. In a preferred embodiment, the monoclonal antibody preferentially binds at least one non-disease-associated allelic variant with at least 10 times greater avidity than it binds at least one disease-associated allelic variant. In another embodiment, the monoclonal antibody preferentially binds to one or more of L-SIGN-3, L-SIGN-4 or L-SIGN-5, and binds less efficiently to L-SIGN-7 or L-SIGN-9. In a preferred embodiment, the monoclonal antibody preferentially binds to L-SIGN-3.

In the methods described herein, detecting and quantifying the binding of an entity may be facilitated by labeling it with a detectable marker. For example, in one embodiment of the instant methods, one or more L-SIGN allelic variants are labeled with a detectable marker. In another embodiment, one or more agents are labeled with a detectable marker. Various types of detectable markers are well known in the art. Such detectable markers include but are not limited to radioactive, calorimetric, chemiluminescent and fluorescent markers.

The present invention also provides an agent that preferentially binds at least one allelic variant of L-SIGN.

Optionally, the agent may be identified with the use of one or more of the methods described above. In one embodiment, the agent preferentially binds at least one disease-associated allelic variant with at least 2 times greater avidity than it binds at least one non-disease-associated allelic variant. In another embodiment, the agent preferentially binds at least one disease-associated allelic variant with at least 5 times greater avidity than it binds at least one non-disease-associated allelic variant. In a preferred embodiment, the agent preferentially binds at least one disease-associated allelic variant with at least 10 times greater avidity than it binds at least one non-disease-associated allelic variant.

In another embodiment, the at least one disease-associated allelic variant is L-SIGN-7 or L-SIGN-9. In a preferred embodiment, the at least one disease-associated allelic variant is L-SIGN-7. In a further embodiment, the at least one non-disease-associated allelic variant is any of L-SIGN-3, L-SIGN-4 or L-SIGN-5. In a preferred embodiment, the at least one non-disease-associated allelic variant is L-SIGN-3.

In yet another embodiment, the agent preferentially binds at least one non-disease-associated allelic variant with at least 2 times greater avidity than it binds at least one disease-associated allelic variant. In a further embodiment, the agent preferentially binds at least one non-disease-associated allelic variant with at least 5 times greater avidity than it binds at least one disease-associated allelic variant. In a preferred embodiment, the agent preferentially binds at least one non-disease-associated allelic variant with at least 10 times greater avidity than it binds at least one disease-associated allelic variant. In another embodiment, the at least one non-disease-associated allelic variant is any of L-SIGN-3, L-SIGN-4 or L-SIGN-5. In a preferred embodiment, the at least one non-disease-associated allelic variant is L-SIGN-3. In a further embodiment, the at least one disease-associated allelic variant is L-SIGN-7 or L-SIGN-9. In a preferred embodiment, the at least one disease-associated allelic variant is L-SIGN-7.

In one embodiment, the agent is an antibody or fragment thereof. In another embodiment, the antibody is a monoclonal antibody. In yet another embodiment, the fragment of the antibody is a fragment of a monoclonal antibody. In a further embodiment, the antibody is a polyclonal antibody. In a still further embodiment, the fragment of the antibody is a fragment of a polyclonal antibody. In another embodiment, the antibody is a humanized antibody or fragment thereof. In a further embodiment, the fragment of the antibody is a fragment of a humanized antibody. In a still further embodiment, the antibody is a human antibody or fragment thereof. In yet another embodiment, the fragment of the antibody is a fragment of a human antibody.

The antibodies of the subject invention may be human or nonhuman antibodies. A nonhuman antibody may be humanized to reduce its immunogenicity in man. Methods for humanizing antibodies are known to those skilled in the art. Various publications, several of which are hereby incorporated by reference into this application, also describe how to make humanized antibodies. For example, methods described in U.S. Pat. No. 4,816,567 enable the production of chimeric antibodies having a variable region of one antibody and a constant region of another antibody. U.S. Pat. No. 5,225,539 describes another approach for the production of a humanized antibody. In this approach, recombinant DNA technology is used to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site directed mutagenesis is used to graft the CDRs onto the framework.

Other approaches for humanizing an antibody are described in U.S. Pat. Nos. 5,585,089 and 5,693,761 and WO 90/07861 which describe methods for producing humanized immunoglobulins. These have one or more CDRs and possible additional amino acids from a donor immunoglobulin and a framework region from an accepting human immunoglobulin. These patents describe a method to increase the affinity of an antibody for the desired antigen. Some amino acids in the framework are chosen to be the same as the amino acids at those positions in the donor rather than in the acceptor. Specifically, these patents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody.

The variable regions of the humanized antibody may be linked to at least a portion of an immunoglobulin constant region of a human immunoglobulin. In one embodiment, the humanized antibody contains both light chain and heavy chain constant regions. The heavy chain constant region usually includes CH1, hinge, CH2, CH3 and sometimes, CH4 regions.

Fully human monoclonal antibodies also can be prepared by immunizing mice transgenic for large portions of human immunoglobulin heavy and light chain loci (see, e.g., U.S. Pat. Nos. 5,591,669; 5,598,369; 5,545,806; 5,545,807; 6,150,584 and references cited therein, the contents of which are incorporated herein by reference). These animals have been genetically modified such that there is a functional deletion in the production of endogenous (e.g., murine) antibodies. These animals are further modified to contain all or a portion of the human germ-line immunoglobulin gene locus such that immunization of these animals results in the production of fully human antibodies. Following immunization of these mice (e.g., XenoMouse®, Abgenix, Fremont, Calif.; HuMab-Mouse®, Medarex/GenPharm, Princeton, N.J.), monoclonal antibodies are prepared according to standard hybridoma technology (e.g., Kohler and Milstein, 1975).

In one embodiment of the agent that preferentially binds at least one allelic variant of L-SIGN, the fragment of the antibody comprises a light chain of an antibody. In another embodiment, the fragment of the antibody comprises a heavy chain of an antibody. In yet another embodiment, the fragment of the antibody comprises an Fab fragment of an antibody. In a further embodiment, the fragment of the antibody comprises an F(ab′)₂ fragment of an antibody. In a still further embodiment, the fragment of the antibody comprises an Fd fragment of an antibody. In one embodiment, the fragment of the antibody comprises an Fv fragment of an antibody. In another embodiment, the fragment of the antibody comprises a variable domain of an antibody. In a further embodiment, the fragment of the antibody comprises one or more CDR domains of an antibody.

In another embodiment, the agent is a peptide. In yet another embodiment, the agent comprises a peptide bond. In a further embodiment, the agent is a non-peptidyl agent. In a still further embodiment, the non-peptidyl agent is a carbohydrate. Such carbohydrate may be any carbohydrate known to one skilled in the art including but not limited to mannose, mannan or methyl-D-mannopyranoside. In another embodiment, the agent is a small molecule or low molecular weight molecule. In a further embodiment, the agent has a molecular weight less than 500 daltons.

In one embodiment, the agent preferentially binds at least one allelic variant of L-SIGN with at least 2 times greater avidity than it binds a related intercellular adhesion molecule-3-grabbing nonintegrin molecule. In another embodiment, the agent preferentially binds at least one allelic variant of L-SIGN with at least 5 times greater avidity than it binds a related intercellular adhesion molecule-3-grabbing nonintegrin molecule. In a preferred embodiment, the agent preferentially binds at least one allelic variant of L-SIGN with at least 10 times greater avidity than it binds a related intercellular adhesion molecule-3-grabbing nonintegrin molecule. In a further embodiment, the at least one allelic variant to which any of the instant agents binds is L-SIGN-7 or L-SIGN-9. In a preferred embodiment, the at least one allelic variant to which any of the instant agents binds is L-SIGN-7. In a still further embodiment, the at least one allelic variant to which any of the instant agents binds is any of L-SIGN-3, L-SIGN-4 or L-SIGN-5. In a preferred embodiment, the at least one allelic variant to which any of the instant agents binds is L-SIGN-3.

An example of an intercellular adhesion molecule-3-grabbing nonintegrin molecule that is related to L-SIGN is DC-SIGN, and it is possible that the identification and characterization of additional intercellular adhesion molecule-3-grabbing nonintegrin molecules will be reported in the future. In further embodiments of the instant agent, the related intercellular adhesion molecule-3-grabbing nonintegrin molecule is DC-SIGN.

In one embodiment of the present invention, any one of the agents described herein preferentially binds at least one non-disease-associated allelic variant with at least 2 times greater avidity than it binds at least one disease-associated allelic variant. In another embodiment, the agent preferentially binds at least one non-disease-associated allelic variant with at least 5 times greater avidity than it binds at least one disease-associated allelic variant. In a further embodiment, the agent preferentially binds at least one non-disease-associated allelic variant with at least 10 times greater avidity than it binds at least one disease-associated allelic variant. In another embodiment, the at least one non-disease-associated allelic variant is any of L-SIGN-3, L-SIGN-4 or L-SIGN-5. In a preferred embodiment, the at least one non-disease-associated allelic variant is L-SIGN-3.

In yet another embodiment, any one of the agents described herein preferentially binds at least one disease-associated allelic variant with at least 2 times greater avidity than it binds at least one non-disease-associated allelic variant. In a further embodiment, the agent preferentially binds at least one disease-associated allelic variant with at least 5 times greater avidity than it binds at least one non-disease-associated allelic variant. In a still further embodiment, the agent preferentially binds at least one disease-associated allelic variant with at least 10 times greater avidity than it binds at least one non-disease-associated allelic variant. In another embodiment, the at least one disease-associated allelic variant is any of L-SIGN-7 or L-SIGN-9. In a preferred embodiment, the at least one non-disease-associated allelic variant is L-SIGN-7.

In another embodiment, the agent is an antibody or fragment thereof. In a further embodiment, the antibody is a monoclonal antibody. In a still further embodiment, the fragment of the antibody is a fragment of a monoclonal antibody. In yet another embodiment, the antibody is a polyclonal antibody. In a further embodiment, the fragment of the antibody is a fragment of a polyclonal antibody. In another embodiment, the antibody is a humanized antibody or fragment thereof. In a further embodiment, the fragment of the antibody is a fragment of a humanized antibody. In a still further embodiment, the antibody is a human antibody or fragment thereof. In yet another embodiment, the fragment of the antibody is a fragment of a human antibody.

In one embodiment of the above-described invention, the fragment of the antibody comprises a light chain of an antibody. In another embodiment, the fragment of the antibody comprises a heavy chain of an antibody. In yet another embodiment, the fragment of the antibody comprises an Fab fragment of an antibody. In a further embodiment, the fragment of the antibody comprises an F(ab′)₂ fragment of an antibody. In a still further embodiment, the fragment of the antibody comprises an Fd fragment of an antibody. In one embodiment, the fragment of the antibody comprises an Fv fragment of an antibody. In another embodiment, the fragment of the antibody comprises a variable domain of an antibody. In a further embodiment, the fragment of the antibody comprises one or more CDR domains of an antibody.

In another embodiment, the agent is a peptide. In yet another embodiment, the agent comprises a peptide bond. In a further embodiment, the agent is a non-peptidyl agent. In a still further embodiment, the non-peptidyl agent is a carbohydrate. Such carbohydrate may be any carbohydrate known to one skilled in the art including but not limited to mannose, mannan or methyl-D-mannopyranoside. In another embodiment, the agent is a small molecule or low molecular weight molecule. In a further embodiment, the agent has a molecular weight less than 500 daltons.

The designing and synthesizing of chemical agents described herein that bind specifically to a receptor such as L-SIGN may be facilitated by experimental approaches that are well known in the art, including traditional medicinal chemistry and the newer technology of combinatorial chemistry, both of which may be supported by computer-assisted molecular modeling. With such approaches, chemists and pharmacologists use their knowledge of the structures of receptor subtypes (in this case allelic L-SIGN variants) and agents determined to bind the receptor to design and synthesize a variety of additional agents that will bind to the receptor subtypes.

Combinatorial chemistry involves automated synthesis of a variety of novel agents by assembling them using different combinations of chemical building blocks. The use of this technique greatly accelerates the process of generating agents. The resulting arrays of agents are called libraries and are used to screen for agents (“lead agents”) that demonstrate a sufficient level of binding at receptors of interest. Using combinatorial chemistry it is possible to synthesize “focused” libraries of agents anticipated to be highly biased toward the receptor target of interest.

Once lead agents are identified, whether through the use of combinatorial chemistry or traditional medicinal chemistry or otherwise, a variety of homologs and analogs are prepared to facilitate an understanding of the relationship between chemical structure, binding affinity for the receptor, and biological or functional activity, which in the methods described herein is the ability of an agent to block the binding of a pathogen to the receptor and thereby inhibit pathogen infection. These studies define structure activity relationships (SARs) which are then used to design drugs with improved potency, selectivity and pharmacokinetic properties. Combinatorial chemistry is also used to rapidly generate a variety of structures for lead optimization. Traditional medicinal chemistry, which involves the synthesis of agents one at a time, is also used for further refinement and to generate agents not synthesizable by automated techniques. Once such drugs are defined, production is scaled up using standard chemical manufacturing methodologies utilized throughout the pharmaceutical and chemical industries.

Numerous non-peptidyl small molecules are available from a variety of commercial sources for screening for agents having desired functional properties. For example, ChemDiv (San Diego, Calif.) has an International Diversity collection of small molecules comprising over 150,000 small molecules selected from more than 3,500,000 chemical agents, and a CombiLab set of over 2,000 libraries of “probe” agents. Each library is represented by the validated template, a set of corresponding building blocks, substituents for SAR synthesis, the off-shelf probe compound set and complete synthetic protocol. Every template is prone.

The total feasible chemistry space of CombiLab's libraries is over 10,000,000,000 structures, with 250,000 of these being represented by probe sets. The major emphases of these libraries are on chemical novelty, drug- and lead-likeness, particular protein families identified as potential therapeutic target, favorable predicted absorption, distribution, metabolism, and excretion (ADME) and toxicity properties, and synthetic feasibility and cost. All compounds are produced in >150 mg quantities by liquid-phase parallel synthesis and individually purified to meet a >90% purity threshold. Every final compound and all key intermediates are analyzed by LC-MS or NMR at 400 Mhz.

ChemDiv has a large number of small molecules that are usable as building blocks for identifying and optimizing the chemical structures of agents in the screening methods described herein. The following are examples of building block molecules available for custom synthesis: 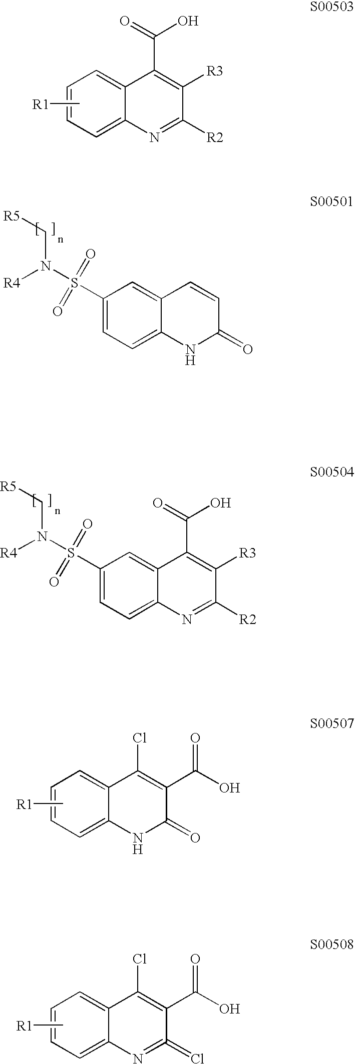
S00501, S00503, S00504: R1=H, CH₃, Cl, CF₃, and other; R2=alkyl, aryl, hetaryl, and other; R2+R3=(CH₂)ₘ; R4, R5=H, alkyl, aryl, heterocyclyl, and other; m=0, 1-4; n=1-3.
S00507, S00508: R=H, alkyl, alkoxy, Cl and other.

The library of building blocks contains scaffolds with several reaction centers, of which the following are examples: 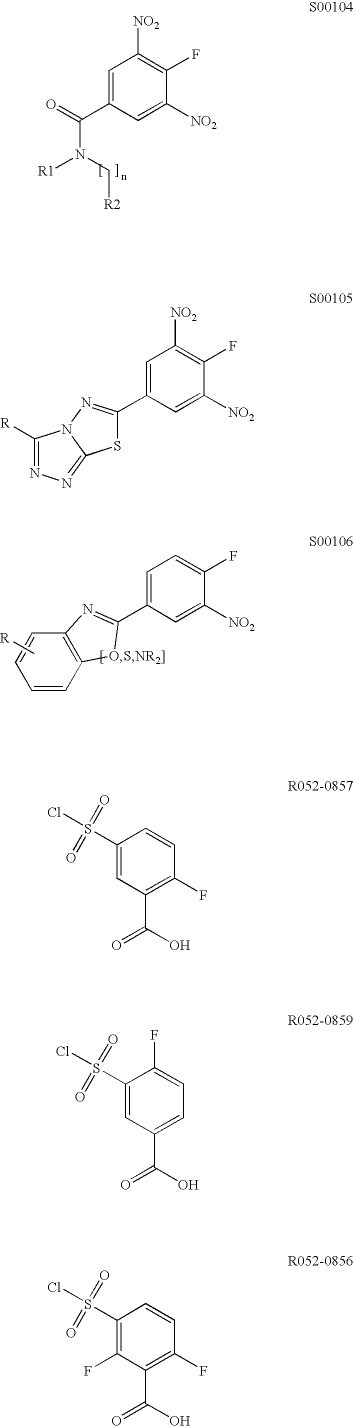
S00104: R1, R2=H, alkyl, aryl, heterocyclyl, and other; n=0, 1-5; R1-N-(CH₂)ₙ-R2=heterocycle.
S00105: R═H, alkyloxyalkyl, alkylthioalkyl, aryl, heterocyclyl, and other.
S00106: R═H, alkyl, alkyloxy, F, Cl, and other.

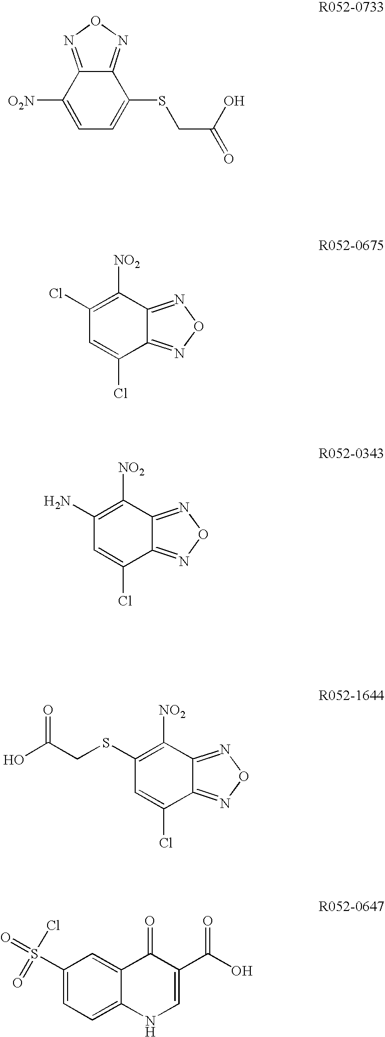

Each of these scaffolds may be used for generating a series of different combinatorial libraries. For example, the scheme below depicts some agents belonging to various combinatorial libraries, which can be produced with the scaffolds containing the fluoronitrobenzene moiety.

Libraries of nonpeptidyl small molecule agents for use in the present invention are also commercially available from Chembridge Collections (ChemBridge Corp., San Diego, Calif.). One ChemBridge library, PHARMACOphore diverse combination library, has over 60,000 compounds comprising multiple, chemically diverse libraries/templates. The average number of compounds per library/template is less than 2,000 with multiple chemical motifs inside each individual library.

Another library available from ChemBridge includes DIVERSet which contains 50,000 compounds. 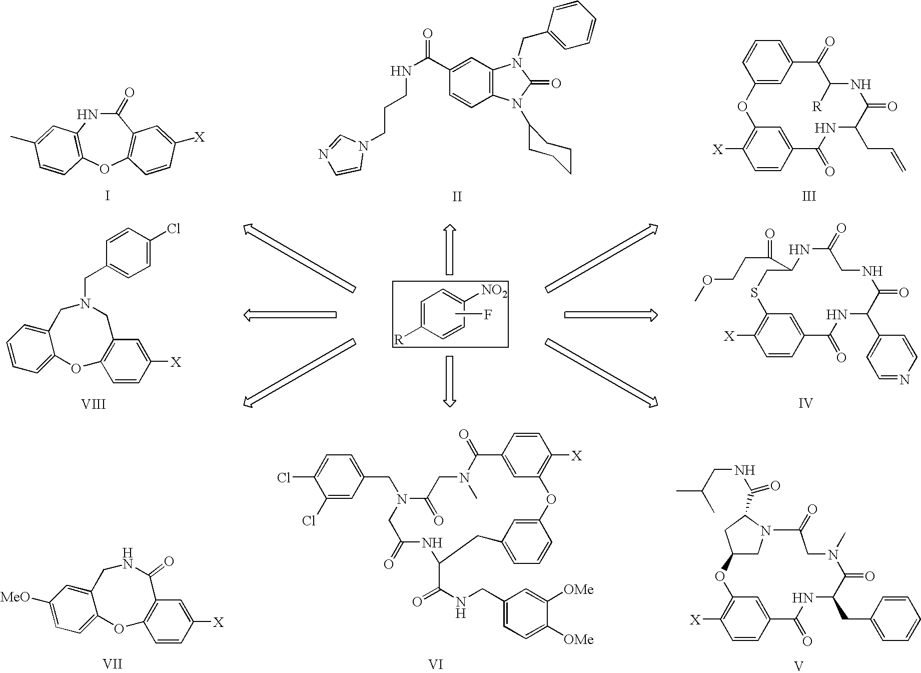
Compound I is described by Ouyang et al. (1999a), VII by Ouyang et al. (1999b) and VIII by Quyang et al. (1999c). Compound II is described by Wei et al. (1998). Compounds III is described by Kiselyov et al. (1999a); IV by Kiselyov et al. (1998) and VI by Kiselyov et al. (1999b). Compound V is described by Goldberg et al. (1999).

This invention also provides a composition comprising any of the agents described herein and a carrier. In one embodiment, the composition further comprises at least one conventional antiviral agent. In a further embodiment, the antiviral agent includes but is not limited to the group consisting of interferon-alpha, interferon-alpha-2B and ribavirin.

Pharmaceutically acceptable carriers are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. In addition to carriers described above, a vaccine may further include carriers known in the art such as, for example, thyroglobulin, albumin, tetanus toxoid, polyamino acids such as polymers of D-lysine and D-glutamate, inactivated influenza virus and hepatitis B recombinant protein(s). The vaccine may also include any well known adjuvants such as incomplete Freund's adjuvant, alum, aluminum phosphate, aluminum hydroxide, monophosphoryl lipid A (MPL, GlaxoSmithKline, Research Triangle Park, N.C.), saponins including QS21 (GlaxoSmithKline), CpG oligonucleotides (Krieg et al., 1995), montanide, vitamin E and various water-in-oil emulsions prepared from biodegradable oils such as squalene and/or tocopherol, Quil A, Ribi Detox, CRL-1005, L-121 and combinations thereof. Preservatives and other additives, such as, for example, antimicrobials, antioxidants, chelating agents, inert gases, and the like may also be included with all the above carriers.

Some of the agents determined using methods described herein to bind preferentially to particular L-SIGN variants in so binding also inhibit the binding of HCV and other pathogens to these variant L-SIGN receptors. For example, the binding of an agent to L-SIGN-7 or L-SIGN-9 polypeptides, which have been shown to efficiently bind HCVpp and to mediate trans-infection, may block the binding of pathogens to these polypeptides. In a preferred embodiment of the invention described herein, the variant polypeptide to which the agent binds is L-SIGN-7. By inhibiting binding of the pathogen to L-SIGN, the agents of the instant invention block host cell-pathogen fusion and, where applicable, entry of the pathogen or its nucleic acid into the host cell. Such agents therefore confer resistance to pathogen infection and have wide therapeutic application in treating subjects afflicted with a pathogen-related disorder and/or in inhibiting the onset of such disorder in a subject. Furthermore, because the L-SIGN genotypes can vary significantly in different individuals and these genotypes can be readily determined, the agents determined herein are also useful for the development of patient-specific therapies. In various methods described herein, such agents are used in therapeutically or prophylactically effective amounts respectively to treat a subject afflicted with a pathogen-related disorder or to inhibit the onset of such a disorder.

Specifically, this invention provides a method for treating a subject afflicted with a pathogen-related disorder, susceptibility to which is associated with at least one polymorphism in L-SIGN, which method comprises administering to the subject an agent, wherein this agent is (1) determined to preferentially bind to at least one allelic variant of L-SIGN using a method comprising: (a) separately contacting an agent with one of (i) at least two allelic variants of L-SIGN, (ii) at least two cell lines expressing allelic variants of L-SIGN, and (iii) plasma membrane fractions from extracts of the at least two cell lines, under conditions suitable for binding of the agent; and (b) comparing the relative binding of the agent to the allelic variants, or to the cells or membrane fractions expressing allelic variants, wherein a difference in relative binding indicates that the agent preferentially binds to at least one allelic variant of L-SIGN; and (2) administered to the subject in a therapeutically effective amount to treat the subject.

This invention also provides a method for treating a subject afflicted with a pathogen-related disorder, susceptibility to which is associated with at least one polymorphism in L-SIGN, which method comprises administering to the subject an agent, wherein this agent is (1) determined to preferentially bind to a first allelic variant of L-SIGN using a method comprising: (a) separately contacting an agent with (i) both a first allelic variant and a second allelic variant of L-SIGN known to bind to the agent, and with (ii) only the second variant, under conditions suitable for binding of the agent to both the first and the second allelic variant; and (b) comparing the extent of binding of the agent to the second variant in the absence versus in the presence of the first variant, wherein a smaller extent of binding of the agent to the second variant in the presence of the first variant indicates that the first variant inhibits the binding of the agent to the second variant, thereby identifying the first allelic variant as one to which the agent preferentially binds; and (2) administered to the subject in a therapeutically effective amount to treat the subject.

The present invention further provides a method for treating a subject afflicted with a pathogen-related disorder, susceptibility to which is associated with at least one polymorphism in L-SIGN, which method comprises administering to the subject a first agent, wherein this first agent is determined to preferentially bind to an allelic variant of L-SIGN using a method comprising: (a) separately contacting one of (i) an allelic variant of L-SIGN, (ii) cells expressing the allelic variant, and (iii) plasma membrane fractions from such cells with (1) both a first agent and a second agent known to bind to the allelic variant, and (2) only the second agent, under conditions suitable for binding of the allelic variant to both the first and the second agent; and (b) comparing the extent of binding of the allelic variant, or cells or membrane fractions expressing the variant, to the second agent in the absence versus in the presence of the first agent, wherein a smaller extent of binding of the allelic variant to the second agent in the presence of the first agent indicates that the first agent inhibits the binding of the allelic variant to the second agent, so as to thereby determine whether the first agent preferentially binds to the allelic variant of L-SIGN; and the first agent is administered to the subject in a therapeutically effective amount to treat the subject.

This invention also provides a method for preventing infection of a subject by a pathogen, susceptibility to which infection is associated with at least one polymorphism in L-SIGN, which method comprises administering to the subject an agent, wherein this agent is (1) determined to preferentially bind to at least one allelic variant of L-SIGN using a method comprising: (a) separately contacting an agent with one of (i) at least two allelic variants of L-SIGN, (ii) at least two cell lines expressing allelic variants of L-SIGN, and (iii) plasma membrane fractions from extracts of the at least two cell lines, under conditions suitable for binding of the agent; and (b) comparing the relative binding of the agent to the allelic variants, or to the cells or membrane fractions expressing allelic variants, wherein a difference in relative binding indicates that the agent preferentially binds to at least one allelic variant of L-SIGN; and wherein the agent is (2) administered to the subject in a prophylactically effective amount to prevent infection by the pathogen.

This invention further provides a method for preventing infection of a subject by a pathogen, susceptibility to which infection is associated with at least one polymorphism in L-SIGN, which method comprises administering to the subject an agent, wherein this agent is (1) determined to preferentially bind to a first allelic variant of L-SIGN using a method comprising: (a) separately contacting an agent with (i) both a first allelic variant and a second allelic variant of L-SIGN known to bind to the agent, and with (ii) only the second variant, under conditions suitable for binding of the agent to both the first and the second allelic variant; and (b) comparing the extent of binding of the agent to the second variant in the absence versus in the presence of the first variant, wherein a smaller extent of binding of the agent to the second variant in the presence of the first variant indicates that the first variant inhibits the binding of the agent to the second variant, thereby identifying the first allelic variant as one to which the agent preferentially binds; and wherein the agent is (2) administered to the subject in a prophylactically effective amount to prevent infection by the pathogen.

This invention still further provides a method for preventing infection of a subject by a pathogen, susceptibility to which infection is associated with at least one polymorphism in L-SIGN, which method comprises administering to the subject a first agent, wherein this first agent is determined to preferentially bind to an allelic variant of L-SIGN using a method comprising: (a) separately contacting one of (i) an allelic variant of L-SIGN, (ii) cells expressing the allelic variant, and (iii) plasma membrane fractions from such cells with (1) both a first agent and a second agent known to bind to the allelic variant, and (2) only the second agent, under conditions suitable for binding of the allelic variant to both the first and the second agent; and (b) comparing the extent of binding of the allelic variant, or cells or membrane fractions expressing the variant, to the second agent in the absence versus in the presence of the first agent, wherein a smaller extent of binding of the allelic variant to the second agent in the presence of the first agent indicates that the first agent inhibits the binding of the allelic variant to the second agent, so as to thereby determine whether the first agent preferentially binds to the allelic variant of L-SIGN; and wherein the first agent is administered to the subject in a prophylactically effective amount to prevent infection by the pathogen.

This invention also provides a method for inhibiting in a subject the onset of a pathogen-related disorder, susceptibility to which is associated with at least one polymorphism in L-SIGN, which method comprises administering to the subject an agent, wherein this agent is (1) determined to preferentially bind to at least one allelic variant of L-SIGN using a method comprising: (a) separately contacting an agent with one of (i) at least two allelic variants of L-SIGN, (ii) at least two cell lines expressing allelic variants of L-SIGN, and (iii) plasma membrane fractions from extracts of the at least two cell lines, under conditions suitable for binding of the agent; and (b) comparing the relative binding of the agent to the allelic variants, or to the cells or membrane fractions expressing allelic variants, wherein a difference in relative binding indicates that the agent preferentially binds to at at least one allelic variant of L-SIGN; and wherein the agent is (2) administered in a prophylactically effective amount to have a prophylactic effect in the subject.

This invention further provides a method for inhibiting in a subject the onset of a pathogen-related disorder, susceptibility to which is associated with at least one polymorphism in L-SIGN, which method comprises administering to the subject a prophylactically effective amount of an agent, wherein this agent is (1) determined to preferentially bind to a first allelic variant of L-SIGN using a method comprising: (a) separately contacting an agent with (i) both a first allelic variant and a second allelic variant of L-SIGN known to bind to the agent, and with (ii) only the second variant, under conditions suitable for binding of the agent to both the first and the second allelic variant; and (b) comparing the extent of binding of the agent to the second variant in the absence versus in the presence of the first variant, wherein a smaller extent of binding of the agent to the second variant in the presence of the first variant indicates that the first variant inhibits the binding of the agent to the second variant, thereby identifying the first allelic variant as one to which the agent preferentially binds; and wherein the agent is (2) administered to the subject in a prophylactically effective amount to have a prophylactic effect in the subject.

This invention still further provides a method for inhibiting in a subject the onset of a pathogen-related disorder, susceptibility to which is associated with at least one polymorphism in L-SIGN, which method comprises administering to the subject a prophylactically effective amount of a first agent, wherein this first agent is determined to preferentially bind to an allelic variant of L-SIGN using a method comprising: (a) separately contacting one of (i) an allelic variant of L-SIGN, (ii) cells expressing the allelic variant, and (iii) plasma membrane fractions from the cells with (1) both a first agent and a second agent known to bind to the allelic variant, and (2) only the second agent, under conditions suitable for binding of the allelic variant to both the first and the second agent; and (b) comparing the extent of binding of the allelic variant, or cells or membrane fractions expressing the variant, to the second agent in the absence versus in the presence of the first agent, wherein a smaller extent of binding of the allelic variant to the second agent in the presence of the first agent indicates that the first agent inhibits the binding of the allelic variant to the second agent, so as to thereby determine whether the first agent preferentially binds to the allelic variant of L-SIGN; and wherein the first agent is administered to the subject in a prophylactically effective amount to have a prophylactic effect in the subject.

In one embodiment of any of the methods described herein, susceptibility of the subject to pathogen infection or to a pathogen-related disorder is associated with the L-SIGN-7 or L-SIGN-9 polymorphism, or a combination thereof. In a preferred embodiment, susceptibility of the subject is associated with the L-SIGN-7 polymorphism.

Determining a therapeutically or prophylactically effective amount of the agents and compositions described herein can be done based on animal data using routine computational methods. The effective amount is based upon, among other things, the size, form, biodegradability, bioactivity and bioavailability of the agent. By way of illustration, if the agent does not degrade quickly, is bioavailable and highly active, a smaller amount will be required to be effective.

In one embodiment of the instant invention, the therapeutically or prophylactically effective amount contains between about 0.000001 mg/kg body weight and about 1000 mg/kg body weight of polypeptide or non-peptidyl agent. In another embodiment, the effective amount contains between about 0.0001 mg/kg body weight and about 250 mg/kg body weight of polypeptide or non-peptidyl agent. In a further embodiment, the effective amount contains between about 0.001 mg/kg body weight and about 50 mg/kg body weight of polypeptide or non-peptidyl agent. In a still further embodiment, the effective amount contains between about 0.01 mg/kg body weight and about 10 mg/kg body weight of polypeptide or non-peptidyl agent. In another embodiment, the effective amount contains between about 0.05 mg/kg body weight and about 2.5 mg/kg body weight of polypeptide or non-peptidyl agent. In yet another embodiment, the effective amount contains between about 0.1 mg/kg body weight and about 0.5 mg/kg body weight of polypeptide or non-peptidyl agent.

The present invention provides a method for preventing infection of a subject by a pathogen, which infection is prevented by immunizing the subject, comprising: (a) administering to the subject one of (i) an allelic L-SIGN protein variant substantially identical to an L-SIGN variant associated with membranes of the subject's cells, and (ii) an expression vector comprising a nucleic acid that encodes the allelic L-SIGN variant protein; so as to thereby (b) elicit in the subject the production of L-SIGN-specific antibodies which inhibit binding of the pathogen to the allelic L-SIGN variant associated with membranes of the subject's cells, wherein these antibodies are not harmful to the subject.

This invention also provides a method for inhibiting in a subject the onset of a pathogen-related disorder, the inhibition of which is effected by immunizing the subject, which method comprises: (a) administering to the subject one of (i) an allelic L-SIGN protein variant substantially identical to an L-SIGN variant associated with membranes of the cells of the subject, and (ii) an expression vector comprising a nucleic acid that encodes the allelic L-SIGN protein variant; so as to thereby (b) elicit in the subject the production of L-SIGN-specific antibodies which inhibit binding of a pathogen to the allelic L-SIGN variant associated with membranes of the subject's cells, wherein these antibodies are not harmful to the subject. Administration of the L-SIGN variant protein or encoding nucleic acid may be performed in several ways, including but not limited to intravenous, intramuscular or subcutaneous injection. In one embodiment of the instant method, the expression vector is formulated prior to administration with an adjuvant, for example a liposome, that mediates entry of the vector into cells wherein expression of the L-SIGN variant occurs.

Embodiments of methods described herein for treating a subject afflicted with a pathogen-related disorder, methods for preventing infection of a subject by a pathogen, and methods for inhibiting in a subject the onset of a pathogen-related disorder, further comprise administration of at least one conventional antiviral agent. In further embodiments, the antiviral agent includes but is not limited to the group consisting of interferon-alpha, interferon-alpha-2B and ribavirin.

Embodiments of methods described above for treating a subject afflicted with a pathogen-related disorder, methods for preventing infection of a subject by a pathogen, and methods for inhibiting in a subject the onset of a pathogen-related disorder, further comprise administration of at least one conventional antimicrobial or antiparasitic agent. In further embodiments, the antimicrobial or antiparasitic agent includes but is not limited to the group consisting of azithromycin, clarithromycin, amikacin, ciprofloxacin, clofazimine, ethambutol, rifabutin, methonidozole, omeprazole, amoxicillin, tetracycline, pentavalent antimony compounds, amphotericin B, pentamidine isoethionate, praziquantel, oxamniquine, metrifonate, chloroquine, azithromycin, and combinations thereof.

This invention further provides a method for predicting resistance of a subject to infection by a pathogen by determining the status of L-SIGN Exon 4 repeat polymorphisms in the subject and correlating that status to a degree of resistance of the subject to the pathogen, which method comprises: (a) extracting genomic DNA from cells of the subject; (b) amplifying the DNA by a polymerase chain reaction (PCR) using primers that are specific for Exon 4 of L-SIGN; (c) identifying the L-SIGN alleles present by determining the size of the amplified DNA, wherein the size of the amplified DNA is proportional to the number of Exon 4 repeats in the allele; and (d) correlating the identity of the L-SIGN alleles in the subject with allelic combinations known to be associated with resistance to infection by the pathogen.

In one embodiment of the instant method, the L-SIGN alleles present in the subject comprise L-SIGN-3, L-SIGN-4 or L-SIGN-5 alleles, or a combination thereof. In a preferred embodiment, L-SIGN alleles present in the subject are L-SIGN-3 alleles. In another embodiment, the pathogen is selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the pathogen is hepatitis C virus (HCV). Resistance to HCV infection may be manifested by a low rate of infection of a subject upon exposure to the virus, low set-point viremia, minimal liver damage, low serum ALT, resolution of acute or chronic infection, and/or low risk of hepatocarcinoma, fibrosis, steatosis, other HCV-related sequelae and/or mortality. In further embodiment of the present invention, the PCR primers used are 5′-TGTCCAAGGTCCCCAGCTCCC-3′ (SEQ ID NO:12) and 5′-GAACTCACCAAATGCAGTCTTCAAATC-3′ (SEQ ID NO:13). In a another embodiment, the size of the amplified DNA is determined by gel electrophoresis.

This invention still further provides a method for predicting susceptibility of a subject to infection by a pathogen by determining the status of L-SIGN Exon 4 repeat polymorphisms in the subject and correlating that status to a degree of susceptibility of the subject to the pathogen, which method comprises: (a) extracting genomic DNA from cells of the subject; (b) amplifying the DNA by a polymerase chain reaction (PCR) using primers that are specific for Exon 4 of L-SIGN; (c) identifying the L-SIGN alleles present by determining the size of the amplified DNA, wherein the size of the amplified DNA is proportional to the number of Exon 4 repeats in the allele; and (d) correlating the identity of the L-SIGN alleles in the subject with allelic combinations known to be associated with susceptibility to infection by the pathogen.

In one embodiment of the instant method, the L-SIGN alleles present in the subject are L-SIGN-7 or L-SIGN-9-alleles, or a combination thereof. In a preferred embodiment, the L-SIGN alleles present in the subject are L-SIGN-7 alleles. In another embodiment, the pathogen is selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the pathogen is hepatitis C virus (HCV). Susceptibility to HCV infection may be manifested by ready infection of a subject upon exposure to the virus, adverse HCV pathogenesis, poor prognosis, elevated serum ALT, and/or high risk of liver damage, hepatocarcinoma, fibrosis, steatosis, other HCV-related sequelae and/or increased risk of mortality. In another embodiment of the present invention, the PCR primers used are 5′-TGTCCAAGGTCCCCAGCTCCC-3′ (SEQ ID NO:12) and 5′-GAACTCACCAAATGCAGTCTTCAAATC-3′(SEQ ID NO:13). In a further embodiment, the size of the amplified DNA is determined by gel electrophoresis.

In addition to Exon 4 repeat polymorphisms, other types of polymorphisms in L-SIGN genes including, for example, single nucleotide polymorphisms (SNPs; see Kobayashi et al., 2002), nucleotide insertions, nucleotide deletions and repeat region polymorphisms such as the presence of variable numbers of tandem repeats outside of Exon 4, may also be used to predict the resistance or suceptibility of a subject to pathogen infection and disease. SNPs are sequence variations that result from single nucleotide alterations in a nucleic acid sequence. They are of great value in biomedical research, disease diagnosis, drug development and pharmacogenetics because they can be used as surrogate markers to locate adjacent genes in genomic DNA involved in causing diseases, and can also themselves be the cause of the disease state if they occur in the coding or regulatory regions of a gene (Gray et al., 2000). As both markers and as polymorphisms with functional consequences, SNPs have the advantage of being highly abundant and relatively stable due to low mutation rates.

SNPs were initially detected by identification of restriction fragment length polymorphisms (Botstein et al., 1980), i.e., the identification of variations in the lengths of DNA fragments generated by the presence or absence of restriction endonuclease cleavage sites. Subsequent methods developed to detect SNPs include heteroduplex analysis and the identification of single strand conformation polymorphisms (SSCPs). Heteroduplex analysis (Lichten and Fox, 1983) relies on the detection of a heteroduplex formed during reannealing of the denatured strands of a PCR product derived from an individual heterozygous for the SNP. The heteroduplex can be detected by anomalous band migration on a gel, or by differential retention on a high-performance liquid chromatography (HPLC) column (Choy et al., 1999). For SSCP detection (Orita et al., 1989), the DNA fragment spanning the putative SNP is amplified by PCR, denatured and run on a non-denaturing polyacrylamide gel. During electrophoresis, the single-stranded fragments adopt sequence-dependent secondary structures and fragments bearing SNPs are identified by their aberrant migration pattern.

As SNPs have assumed greater importance in biomedical research, a variety of high-throughput methods of detecting them have recently been developed. One common method is direct DNA sequencing using automated DNA sequencers. Another technique which has rapidly gained popularity is variant detector arrays (VDAs) (Wang et al., 1998). This technique allows the identification of SNPs by hybridization of a PCR product to oligonucleotides arrayed on a glass chip and measuring the difference in hybridization strength between matched and mismatched oligonucleotides.

This invention also provides a method for predicting resistance of a subject to infection by a pathogen by identifying single nucleotide L-SIGN polymorphisms in the subject and correlating the presence of these single nucleotide polymorphisms (SNPs) to the resistance of the subject to the pathogen, which method comprises: (a) extracting genomic DNA from cells of a subject; (b) amplifying the genomic DNA by a polymerase chain reaction (PCR) using primers that are specific for regions within L-SIGN alleles; (c) screening the amplified DNA to detect SNPs; and (d) correlating the identity of alleles containing SNPs so detected with allelic combinations known to be associated with resistance to infection by the pathogen. In one embodiment, the SNPs are detected by identification of single strand conformation polymorphisms (SSCPs). In another embodiment, the SNPs are detected by heteroduplex analysis. In a further embodiment, the SNPs are detected by direct DNA sequencing. In a still further embodiment, the SNPs are detected by variant detector arrays (VDAs).

In another embodiment of the instant method, the pathogen is selected from a group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the pathogen is hepatitis C virus (HCV)

This invention further provides a method for predicting the susceptibility of a subject to infection by a pathogen by identifying single nucleotide L-SIGN polymorphisms in the subject and correlating the presence of these single nucleotide polymorphisms (SNPs) to the susceptibility of the subject to the pathogen, which method comprises: (a) extracting genomic DNA from cells of a subject; (b) amplifying the genomic DNA by a polymerase chain reaction (PCR) using primers that are specific for regions within L-SIGN alleles; (c) screening the amplified DNA to detect SNPs; and (d) correlating the identity of alleles containing SNPs so detected with allelic combinations known to be associated with susceptibility to infection by the pathogen. In one embodiment, the SNPs are detected by identification of single strand conformation polymorphisms (SSCPs). In another embodiment, the SNPs are detected by heteroduplex analysis. In a further embodiment, the SNPs are detected by direct DNA sequencing. In a still further embodiment, the SNPs are detected by variant detector arrays (VDAs).

In another embodiment of the instant method, the pathogen is selected from a group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the pathogen is hepatitis C virus (HCV).

This invention also provides a method for predicting resistance of a subject to infection by a pathogen by identifying an L-SIGN polymorphism in the subject other than a single nucleotide polymorphism (SNP) or an Exon 4 repeat polymorphism, and correlating the presence of this L-SIGN polymorphism to the resistance of the subject to the pathogen, which method comprises: (a) extracting genomic DNA from cells of a subject; (b) amplifying the genomic DNA by a polymerase chain reaction (PCR) using primers that are specific for regions of L-SIGN; (c) sequencing the amplified DNA and comparing the sequence to known sequences of L-SIGN alleles to detect any polymorphisms present; and (d) correlating the identity of alleles containing a detected polymorphism, wherein this polymorphism is not a SNP or an Exon 4 repeat polymorphism, with allelic combinations known to be associated with resistance to infection by the pathogen.

In one embodiment of this method, the pathogen is selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the pathogen is hepatitis C virus (HCV).

This invention further provides a method for predicting susceptibility of a subject to infection by a pathogen by identifying an L-SIGN polymorphism in the subject other than a single nucleotide polymorphism (SNP) or an Exon 4 repeat polymorphism, and correlating the presence of this L-SIGN polymorphism to the susceptibility of the subject to the pathogen, which method comprises: (a) extracting genomic DNA from cells of a subject; (b) amplifying the genomic DNA by a polymerase chain reaction (PCR) using primers that are specific for regions of L-SIGN; (c) sequencing the amplified DNA and comparing the sequence to known sequences of L-SIGN alleles to detect any polymorphisms present; and (d) correlating the identity of alleles containing a detected polymorphism, wherein this polymorphism is not a SNP or an Exon 4 repeat polymorphism, with allelic combinations known to be associated with susceptibility to infection by the pathogen. In one embodiment of this method, the pathogen is selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the pathogen is hepatitis C virus (HCV).

This invention also provides an article of manufacture comprising a packaging material containing therein an agent identified by any one of the methods described herein and a label providing instructions for using the agent to treat a subject afflicted with a pathogen-associated disorder. In one embodiment, the disorder is associated with a pathogen selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the disorder is associated with hepatitis C virus (HCV).

This invention further provides an article of manufacture comprising a packaging material containing therein an agent as described herein and a label providing instructions for using the agent to prevent infection of a subject by a pathogen. In one embodiment, the pathogen is selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the pathogen is hepatitis C virus (HCV).

This invention still further provides an article of manufacture comprising a packaging material containing therein an agent identified by any one of the methods described herein and a label providing instructions for using the agent to inhibit the onset of a pathogen-associated disorder in a subject. In one embodiment, the disorder is associated with a pathogen selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the disorder is associated with hepatitis C virus (HCV).

This invention additionally provides an article of manufacture comprising a packaging material containing therein one of (i) an allelic L-SIGN protein variant substantially identical to an L-SIGN variant associated with membranes of cells of a subject, and (ii) an expression vector comprising a nucleic acid that encodes this allelic L-SIGN protein variant, and a label providing instructions for using the L-SIGN variant protein or expression vector to prevent infection of the subject by a pathogen, which infection is prevented by using the L-SIGN protein variant as an immunogen to immunize the subject. In one embodiment, the pathogen is selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the pathogen is hepatitis C virus (HCV).

This invention also provides an article of manufacture comprising a packaging material containing therein one of (i) an allelic L-SIGN protein variant substantially identical to an L-SIGN variant associated with membranes of cells of a subject, and (ii) an expression vector comprising a nucleic acid that encodes this allelic L-SIGN protein variant, and a label providing instructions for using the L-SIGN variant protein or expression vector to inhibit in a subject the onset of a pathogen-related disorder, the inhibition of which is effected by using the L-SIGN protein variant as an immunogen to immunize the subject. In one embodiment, the disorder is associated with a pathogen selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species. In a preferred embodiment, the disorder is associated with hepatitis C virus (HCV).

Experimental Details

The following Experimental Details are set forth to aid in an understanding of the invention, and are not intended, and should not be construed, to limit in any way the invention set forth in the claims which follow thereafter.

Materials and Methods

Plasmids and Transfected Cell Lines

The pcDNA3-DC-SIGN and pcDNA3-DC-SIGN-related plasmids are obtained from the National Institutes of Health (NIH) AIDS Research and Reference Reagent Program. The NLenv-luc+ vector is described in Connor et al. (1995). pcDNA3.1-E1-E2 is a construct containing unmodified HCV envelope glycoprotein genes, including the putative intron in E1, cloned into the pcDNA3.1 expression vector (Invitrogen, Carlsbad, Calif.). The bicistronic HIV-1-based vector, pLenti6, is available from Invitrogen. DNA encoding the 3-, 4-, 5- and 9-repeat forms of L-SIGN was chemically synthesized (DNA 2.0, Menlo Park, Calif.), ligated into pcDNA3.1, and sequence verified.

Plasmids pcDNA3-DC-SIGN and pcDNA3-DC-SIGN-related were transfected into HeLa cells using a lipid formulation, Effectene™ (Qiagen, Valencia, Calif.), according to the manufacturer's suggested protocol. Two days post-transfection, cells were treated with standard growth media comprising Dulbecco's modified Eagle's medium (DMEM) containing 10% heat-inactivated fetal bovine serum (FBS; HyClone, South Logan, Utah), penicillin/streptomycin (Life Technologies, Carlsbad, Calif.) and L-glutamine (Life Technologies) supplemented with 600 μg/ml geneticin (Life Technologies). After 2 weeks, surviving colonies were selected, expanded, and screened for expression by flow cytometry using monoclonal antibodies that recognize DC-SIGN (120507), L-SIGN (120612), or both DC- and DC-SIGN-R (120604). The transfected HeLa cell lines were routinely cultured in DMEM supplemented with 10% FBS, penicillin/streptomycin, L-glutamine with geneticin (600 μg/ml). Growing cells were divided for maintenance culture using cell dissociation solution (Sigma, St. Louis, Mo.). High expressors were cloned and passaged in growth media with geneticin (600 μg/ml).

Plasmids pcDNA3-DC-SIGN and pcDNA3-DC-SIGN-related are also transfected into murine 3T3 fibroblasts (adherent) and murine L1.2 T cell hybridomas (non-adherent). Methods of transfecting cells with nucleic acid encoding viral protein(s) to obtain cells in which that protein(s) is expressed are well known in the art (see, for example, Gardner et al., 2003; Schulke et al., 2002). Such transfected cells may be used to test chemical agents and screen libraries of chemical agents to obtain agents that bind receptors as well as agents that activate or inhibit activation of functional responses in such cells, and therefore are likely to do so in vivo.

A broad variety of host cells can be used to study heterologously expressed proteins. In addition to HeLa cells, murine 3T3 fibroblasts and L1.2 T cell hybridomas, these cells include, but are not limited to, mammalian cell lines such as Chinese hamster ovary (CHO), COS-7, HEK293, 293T (SV40 large T antigen) and LM(tk⁻), mouse Y1 cells; insect cell lines such as Sf9, Sf21 and Trichoplusia ni 5B-4 cells; amphibian cells such as Xenopus oocytes and Xenopus melanophore cells; assorted yeast strains; assorted bacterial cell strains; and others. Culture conditions for each of these cell types is specific and is known to those familiar with the art.

DNA encoding proteins to be studied can be transiently expressed in a variety of mammalian, insect, amphibian, yeast, bacterial and other cells lines by several transfection methods including, but not limited to, lipid formulation (e.g., Effectene™)-mediated, calcium phosphate-mediated, DEAE-dextran-mediated, liposome-mediated, viral-mediated, electroporation-mediated, and microinjection delivery. Each of these methods may require optimization of experimental parameters depending on the DNA, cell line, and the type of assay to be subsequently employed.

Heterologous DNA can be stably incorporated into host cells, causing the cell to perpetually express a foreign protein. Methods for the delivery of the DNA into the cell are similar to those described above for transient expression but require the co-transfection of an ancillary selectable marker gene to confer a selectable phenotype, e.g., drug resistance, to the targeted host cell. The ensuing drug resistance can be exploited to select and maintain cells that have taken up the DNA. A variety of resistance genes are available including, but not restricted to, kanamycin, geneticin, methotrexate and hygromycin.

Development of Anti-E2 Monoclonal Antibodies

293T cells were transiently transfected with the pcDNA3.1-E1-E2 plasmid. E1 and E2 expression was confirmed by flow cytometry and cells were frozen. (Unmodified E1, containing a putative intron within the coding sequence, is detectable on the cell surface by flow cytometry after labeling with anti-E1 mAb, A4 (provided by Dr. Jean Dubuisson; see Dubuisson et al., 1994). Thawed aliquots of ˜10⁷ E1/E2-expressing 293T cells were used to intraperitoneally immunize female Balb/c mice four times at 3-week intervals. In general, immunological adjuvants are not required to elicit antibody using this approach. The mice were administered an intravenous boost of soluble E2 protein (Austral Biologicals, San Ramon, Calif.) three days prior to splenectomy. Serial dilutions of pre-immune and immune sera (1-1:1000) from week eight were screened by ELISA to confirm specific binding of immune serum antibodies to antigen.

Hybridomas were generated by standard methods, and hybridoma supernatants were similarly screened by ELISA. Briefly, 293T or 293T transiently expressing E1/E2 were solubilized with M-PER® lysis buffer (Pierce, Rockford, Ill.), which conserves membrane protein conformation. 96-well microtiter plates were incubated with GNA lectin, blocked with 5% BSA, and coated with cell lysates or soluble E2 protein (Austral Biologicals). Hybridoma supernatants, anti-E2 MAb H53 (positive control), anti-CD4 MAb Leu3A (negative control) and PBS were added to wells, followed by alkaline phosphatase (AP)-conjugated goat anti-mouse IgG and PNPP-DEA substrate solution (Pierce). The reaction was stopped with EDTA and absorbance measured at 405 nM with reference at 650 nM (A₆₅₀), using a SpectraMax® plate reader (Molecular Devices, Sunnyvale, Calif.).

The mAbs were also characterized by flow cytometry and by western blotting. HeLa-E1*-E2* cells (i.e., HeLa cells expressing “intronless” E1 and E2 sequences from which putative splice acceptor sites had been eliminated) were incubated with hybridoma supernatants followed by a phycoerythrin (PE)-conjugated goat anti-mouse IgG, and analyzed by flow cytometry. For western blot analysis, cells were lysed in 1% NP40 buffer, proteins were separated by SDS-PAGE and transferred to nitrocellulose membranes. Membranes were probed with different mAbs, followed by HRP-labeled goat anti-mouse IgG and incubation with a chemifluorescent substrate.

Fluorescence-Activated Cell Sorter (FACS) Analysis of L-SIGN and DC-SIGN Expression

Cell-surface expression of different L-SIGN isoforms on transfected cell lines was measured using flow cytometry after labeling with specific antibodies. Cells were harvested with cell dissociation solution (Sigma), and FACS analysis was performed as described (Gardner et al., 2003). Hep3B cells were obtained from the American Type Culture Collection (Manassas, Va.). The same methods may be used to measure expression of DC-SIGN.

Antibodies

Monoclonal antibodies (mAbs) to the lectin-binding domain of DC-SIGN (mAb 120507) and L-SIGN (mAb 120604) or both lectins (mAb 120612) were obtained from R & D Systems (Minneapolis, Minn.). MAb 120507 is a DC-SIGN-specific, lectin binding domain-targeted, conformation-dependent, mouse IgG2b. 120507 blocks SIV and HIV infection and ICAM-3 adhesion (Jameson et al., 2002; Wu et al., 2002).

MAb 120604 is an L-SIGN-specific, lectin binding domain-targeted, conformation-dependent, mouse IgG2b. 120604 does not block binding to SIV or HIV, and exhibits only weak or no blocking of ICAM-3 adhesion (Jameson et al., 2002; Wu et al., 2002).

MAb 120612 is a mouse IgG2a that recognizes the lectin binding domain of both DC-SIGN and L-SIGN. 120612 blocks ICAM-3 adhesion and HIV infection (Jameson et al., 2002; Wu et al., 2002).

MAbs DC6 and DC28 (AIDS Research and Reference Reagent Program, Rockville, Md.) recognize the repeat regions of DC-SIGN (DC6) or DC-SIGN and L-SIGN (DC28). DC6 is a mouse IgG1 that recognizes both DC-SIGN and L-SIGN via the neck or repeat region and not the lectin-binding domain. DC6 does not block ICAM-3 binding or SIV transmission (Baribaud et al., 2001).

DC28 is a mouse IgG2a that recognizes both DC-SIGN and L-SIGN via the neck or repeat region and not the lectin-binding domain. DC28 does not block ICAM-3 binding or SIV transmission (Baribaud et al., 2001).

The anti-HCV E2 mAb, 091a-5, was purchased from Austral Biologicals.

The anti-CD81 mAb, JS-81, was obtained from Pharmingen (San Diego, Calif.).

Preparation of Monocyte-Derived Dendritic Cells

Monocyte-derived dendritic cells (MDDC) are prepared from primary human hepatocytes, obtained from Dr. S. Strom, University of Pittsburgh, through the NIH's Liver Tissue Procurement and Distribution System (LTPADS) Program. Human livers are perfused with collagenase, and explanted hepatocytes are filtered and centrifuged. Purified hepatocytes are plated at 50-70% confluency on collagen I-treated tissue culture plates in serum-free Williams' E medium (Ferrini et al., 1997). They are shipped by FedEx the next day and used immediately upon arrival. Whole human blood is purchased from the New York Blood Bank and peripheral blood mononuclear cells (PBMC) are isolated by Ficoll-hypaque density gradient low-speed centrifugation. Either unstimulated bulk PBMC or purified, unstimulated subpopulations are infected. B lymphocytes, T lymphocytes and monocytes are purified using magnetic DynaBeads® (Dynal Biotech Inc., Lake Success, N.Y.) coupled to antibodies against cell-specific antigens, according to the manufacturer's instructions. Monocytes are plated on poly-L-lysine treated plates and cultured for seven days in medium containing autologous donor serum prior to infection. Culturing monocytes in GM-CSF/IL-4-supplemented medium for 3-7 days generates immature MDDC. These are typically MHC class II+ (intermediate level), CD1+, CD14−, CD80/CD86+ (low to intermediate level) and CD83−. To generate mature MDDC (DC0 phenotype), a maturation status is added for the last 1-2 days of culture. The most commonly used are LPS, TNF-α, or a cocktail of cytokines (TNF-α, IL-1 beta, IL-6 and PGE2). The phenotype of these mature DCs is MHC class II (high level), CD1+, CD14−, CD80/CD86+ (high level) and CD83+.

RNA Extraction

Viral RNA is extracted from cells by using a QIAmp Viral RNA Mini Spin kit (Qiagen) with modifications. Briefly, two extractions with 280 μl of lysis buffer are added per well and transferred to a 1.7-ml tube. The empty plate is washed with 140 μl of Dulbecco's phosphate-buffered saline with calcium and magnesium, and pooled into the same tube. RNA extraction and binding to spin columns are carried out according to the manufacturer's instructions. Following a wash with wash buffer, contaminating DNA on the column is removed by treatment with RNase-free DNase (Qiagen) according to the manufacturer's instructions. The bound RNA is washed and eluted from the column in two steps using 30 μl and 40 μl elution buffer respectively, and the eluates are combined.

Quantitation of Viral RNA

The number of HCV RNA copies is determined using the HCV QuantaSure Plus™ real-time PCR assay (LabCorp, Research Triangle Park, N.C.), which has been demonstrated to be sensitive, specific to HCV, and has a linear dynamic range of 10¹-10⁸ copies/ml (Gardner et al., 2003). Briefly, a 4-μl aliquot of extracted RNA is added to a one-step RT-PCR reaction mixture containing sense and antisense primers specific for HCV and a TaqMan probe (proprietary sequences; LabCorp). The cycle at which the amplification plot crosses the threshold is defined as the threshold cycle (C_{T}) and is predictive of the number of HCV RNA copies in the sample. A standard curve is generated for quantification by using serial 10-fold dilutions of a reference HCV (RNA+) sample.

HIV-1 RNA is quantified by PCR using the COBAS Amplicor HIV-1 Monitor®D test (Roche Molecular Systems, Somerville, N.J.), which has a linear dynamic range of 5×10¹-7.5×10⁵ copies/ml (Berger et al., 2002). A standard curve is generated using serial ten-fold dilutions of a reference HIV-1 (RNA+) sample.

SDS-PAGE, Blue Native PAGE, and Western Blot Analyses

Sodium dodecyl sulfate-polyacrylamide gel electrophoresis (SDS-PAGE) is performed as described (Binley et al., 2000). Reduced and nonreduced samples are prepared by boiling for 2 min in Laemmli sample buffer (62.5 mM Tris-HCl [pH 6.8], 2% SDS, 25% glycerol, 0.01% bromophenol blue) in the presence or absence, respectively, of 50 mM dithiothreitol (DTT). Protein purity is determined by densitometric analysis of the stained gels followed by the use of ImageQuant™ software (Molecular Devices, Sunnyvale, Calif.).

Blue Native (BN)-PAGE is carried out with minor modifications to the originally published method (Schagger and von Jagow, 1991; Schagger et al., 1994) as described by Schulke et al. (2002). Purified protein samples or cell culture supernatants are diluted with an equal volume of a buffer containing 100 mM 4-(N-morpholino)propane sulfonic acid (MOPS), 100 mM Tris-HCl, pH 7.7, 40% glycerol, 0.1% Coomassie blue, just prior to loading onto a 4 to 12% Bis-Tris NuPAGE® gel (Invitrogen). Typically, gel electrophoresis is performed for 2 h at 150 V (−0.07 A) using 50 mM MOPS, 50 mM Tris, pH 7.7, 0.002% Coomassie blue as cathode buffer, and 50 mM MOPS, 50 mM Tris, pH 7.7 as anode buffer. When purified proteins are analyzed, the gel is destained with several changes of 50 mM MOPS, 50 mM Tris, pH 7.7 subsequent to the electrophoresis step. Typically, 5 μg of purified protein are loaded per lane.

For western blot analyses, gels and polyvinylidene difluoride (PVDF) membranes are soaked for 10 min in transfer buffer (192 mM glycine, 25 mM Tris, 0.05% SDS [pH 8.8] containing 20% methanol). Following transfer, PVDF membranes are destained of Coomassie blue dye using 25% methanol and 10% acetic acid and air dried. Destained membranes are probed with the mAb followed by horseradish peroxidase (HRP)-labeled anti-mouse immunoglobulin G (IgG) (Kirkegaard & Perry Laboratories, Gaithersburg, Md.), each used at a final concentration of 0.2 μg/ml. The envelope glycoproteins are visualized using the Renaissance western blot Chemiluminescence Reagent Plus system (Perkin-Elmer Life Sciences, Boston, Mass.). Bovine serum albumin (BSA), apo-ferritin, and thyroglobulin are obtained from Amersham Biosciences (Piscataway, N.J.) and used as molecular mass standards.

Quantification of HCV E2 Glycoprotein by Capture ELISA

This procedure for quantifying envelope glycoproteins associated with viral particles is done according to Lu and Kielian (2000). Briefly, virus-producing cells are treated with Sulfo-NHS-LC-Biotin and supernatants collected a day later. Biotinylated pseudovirions are ultracentrifuged on sucrose cushions, resuspended and denatured by heating. Streptavidin-coated ELISA plates are incubated with several serial dilutions of denatured pseudovirions. Captured E2 is detected with a cocktail of anti-E2 mAbs that recognize linear epitopes, including mAbs 23, 26 and 32 (see Results, Anti-E2 monoclonal antibodies), followed by incubation with HRP-conjugated anti-mouse IgG antibody. Optical density is measured at 450 nm using the ImmunoPure® TMB Substrate kit (Pierce). A standard curve is generated using biotinylated, soluble E2 protein (Austral Biologicals). Standardized viral stocks are cryopreserved in 1% sucrose, 1% BSA in phosphate-buffered saline.

Pseudotyping of Retroviral Particles by Native HCV Envelope Glycoproteins

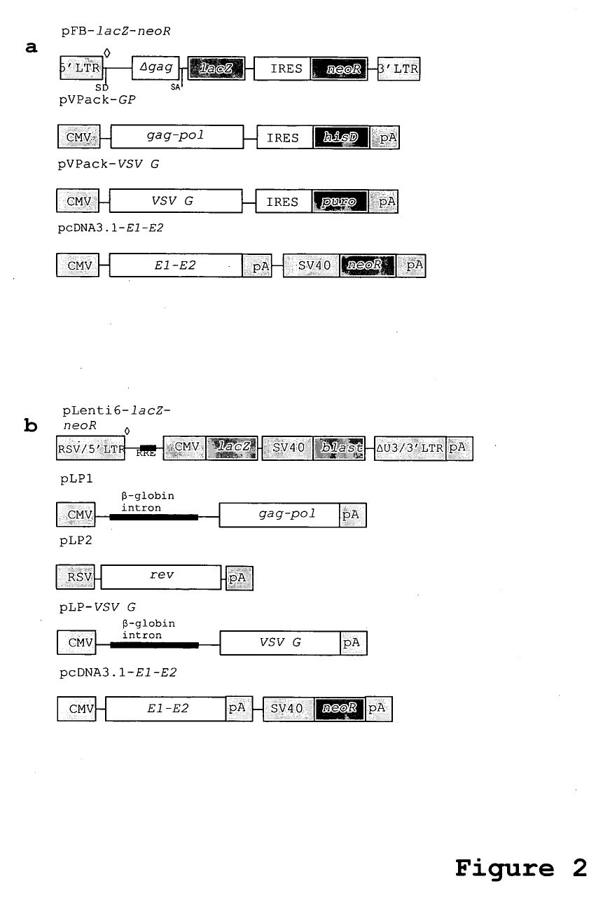

The procedure of Bartosch et al. (2003b) was used to generate retroviral particles pseudotyped with HCV envelope glycoproteins (HCV pseudovirus particles; HCVpp). Briefly, in order to generate HCV pseudovirions, 293T cells were cotransfected with HIV-1-based (pLenti6, Invitrogen) or MMLV-based (pFB, Stratagene, La Jolla, Calif.) retroviral vectors and vectors expressing structural genes, including Gag, Pol and envelope glycoproteins (see FIG. 2). In addition, the HIV-1-based vector was cotransfected with a Rev-encoding plasmid to enable transport of genomic RNAs from the nucleus to the cytoplasm.

HCVpp were also produced by cotransfecting 293T cells with the NIluc+env- reporter vector (Connor et al., 1995) and pcDNA3.1-ΔC-E1-E2 (Dumonceaux et al., 2003) as previously described (Cormier et al., 2004a). NIluc+env- encodes an HIV-1_{NL4.3} envelope-deficient genome expressing luciferase instead of nef. Construct pcDNA3.1-ΔC-E1-E2 encodes full-length E1 and E2 envelope glycoproteins (amino acids 132-746) of HCV isolate H77 (Kolykhalov et al., 1997) starting with the last sixty amino acids of the capsid (ΔC). Putative splice acceptor sites were modified by conservative mutagenesis as described (Dumonceaux et al., 2003). Cell culture supernatants, containing HIV-1 particles pseudotyped with HCV E1/E2 envelope glycoproteins were collected 48 h post-transfection and cleared of cellular debris by low-speed centrifugation.

L-SIGN and DC-SIGN Capture of HCV Pseudovirions Comprising Envelope Glycoprotein of Primary Isolates

Murine 3T3 fibroblasts and murine L1.2 T cell hybridomas are engineered to express L-SIGN and DC-SIGN. Labeling with specific antibodies and analysis by flow cytometry are used to confirm similar expression levels of L-SIGN and DC-SIGN by different cells.

First, the use of HCV pseudovirions in capture assays is validated by confirming that they bind to L-SIGN+ and DC-SIGN+ similarly to naturally occurring HCV virions. To this end, capture of HCV virions from sera of patients with viral loads ≧10⁵ IU/ml is quantified. Serum samples are diluted to 10⁵ IU/ml, as determined by RNA copy numbers, and serial ten-fold dilutions, ranging from 10²-10⁵ IU/ml are incubated with L-SIGN (allele 7)+, DC-SIGN+ and parental cells. After washing, cell-associated viral RNA is extracted (QIAmp Viral RNA Mini Spin Kit, Qiagen) and quantified by real-time PCR (HCV QuantaSure Plus™ assay, LabCorp).

Next, it is determined whether L-SIGN and DC-SIGN binding of naturally occurring HCV virions is quantitatively reflected by binding of HCV pseudovirions bearing E1/E2 derived from matching patient sera. As specificity controls, VSV G pseudotypes, which are not captured by L-SIGN and DC-SIGN, and HIV-1 pseudotypes, which are captured by both SIGN molecules, are included. In addition, binding is inhibited with mannan and mAbs against the lectin domains of L-SIGN and DC-SIGN. Pseudovirions, for use in binding assays only, are generated with the HIV-1-based NLenv-luc+ vector (Connor et el., 1995) in order to facilitate genomic RNA quantification using the Amplicor HIV-1 Monitor® test (Roche). Sequences hybridizing to PCR primers used in this assay are present in the recombinant NLenv-luc+ genome, which does not express HIV-1 envelope glycoproteins due to a frameshift mutation (Barlow et al., 1997). Pseudovirions are purified and standardized for E2 content by capture ELISA. Viral preparations that have similar RNA:E2 ratios are diluted to 10⁵ IU/ml, based on RNA copy numbers. Serial ten-fold dilutions, ranging from 10²-10⁵ IU/ml of pseudoviral suspensions are incubated with L-SIGN+, DC-SIGN+ and parental cells. After washing, cell-associated viral RNA is extracted (QIAmp Viral RNA Mini Spin Kit, Qiagen) and quantified (Amplicor HIV-1 Monitor® test, Roche).

L-SIGN+, DC-SIGN+ and parental cells are incubated with serial ten-fold dilutions of purified and standardized HCV pseudovirion suspensions and cell-associated viral RNA is quantified (Amplicor HIV-1 Monitor® test, Qiagen) Similarly, it is determined whether MDDC, which naturally express DC-SIGN, differentially bind HCV pseudovirions coated with E1/E2 from master quasispecies in serum, PBMC and liver. Binding is normalized for donor-specific differences in DC-SIGN expression, which is analyzed by flow cytometry after specific antibody labeling. Mature MDDC have an unbiased “DC0” phenotype (Sanders et al., 2002).

HCV Pseudovirion Entry into Target Cells Mediated in trans by L-SIGN and DC-SIGN

For these experiments, HIV-1-based lacZ-expressing virions are pseudotyped with E1/E2 from serum-, PBMC- and liver-derived isolates. Viruses are concentrated and purified by ultracentrifugation, and standardized for E2 content.

Transfectants expressing L-SIGN or DC-SIGN, as well as parental cells, are pulsed with serial ten-fold dilutions of HCV pseudovirion suspensions with a range of E2 concentrations. The lower end of this range comprises suboptimal viral concentrations that yield little or no signal in the blue cell assay. Mature (DC0) and immature MDDC are also separately pulsed with several dilutions of HCV pseudovirions. After washing to remove unbound virus, pulsed cells are incubated with non-permissive cells such as 3T3 or BHK-21, as well as permissive hepatic, B-, T-, and endothelial cell lines. In addition, HCV pseudovirion pulsed cells are tested for their ability to promote infection of primary cells such as hepatocytes, B-lymphocytes and T-lymphocytes. As controls, target cells are also directly infected with the same range of concentrations with which L-SIGN+ and DC-SIGN+ cells were pulsed. Cells are assayed for β-galactosidase activity 72 h post-infection by in situ histochemical staining. The cells are first fixed with 0.5% glutaraldehyde and then incubated with 0.4 mg/ml X-gal substrate (5-bromo-4-chloro-3-indolyl-β-D-galactopyranoside) in 5 mM K₃Fe(CN)₆/5 mM K₄Fe(CN)₆, 1 mM MgCl₂. Blue cells are counted after overnight incubation at 37° C.

In all of the experiments, the ability of mannan and mAbs to inhibit HCV pseudotype binding to L-SIGN+ and DC-SIGN+ cells and transmission to target cells is systematically tested. As further specificity controls, VSV G pseudotypes, which are not captured by L-SIGN and DC-SIGN, and HIV-1 pseudotypes, which are captured by both SIGN molecules and transmitted to target cells in trans, are also included. (HIV-1 entry is only measured in T-cell lines and T-lymphocytes which express CD4 and CXCR4.)

E2 and HCVpp Binding Assays

Parental HeLa or HeLa-SIGN transfectants were cultured and harvested as described above and dispensed into assay tubes at 1.5×10⁵ cells/tube. Cells were washed once with staining buffer [Dulbecco's phosphate buffered saline (Invitrogen), 0.25% bovine serum albumin (Sigma, St. Louis, Mo.), and 0.1% sodium azide] and blocked with 10% heat-inactivated goat serum (Invitrogen) in staining buffer for 15 min at 4° C. Purified HCV E2 antigen was added to a final concentration of 5 μg/ml for 1 h at 4° C. Cells were washed and incubated with anti-HCV E2 mAb 091a-5 followed by FITC-conjugated goat anti-mouse IgG (Caltag, Burlingame, Calif.) for analysis by flow cytometry. Binding of HCVpp was measured as previously described (Cormier et al., 2004b).

Trans-Infection and Inhibition

Parental HeLa cells and HeLa-SIGN transfectants (2×10⁴) were incubated with 200 μl of viral supernatant for 2 h at 37° C. Cells were washed three times with serum-free medium to remove unbound virus and then cocultured with Hep3B target cells (4×10⁴) for an additional 48 h at 37° C. as previously described (Cormier et al., 2004b). Luciferase activity was measured in cell lysates using the Luciferase Assay System (Promega, Madison, Wis.) according to the manufacturer's instructions. For inhibition studies, parental and transfected HeLa cells were incubated with anti-L-SIGN mabs (10 μg/ml) or mannan (20 μg/ml) for 30 min at 37° C. prior to addition of viral supernatants. Data were analyzed for statistical significance using 2-sided t-tests and a significance level of P<0.05 unless otherwise indicated. Analyses were performed using JMP software (SAS Institute, Inc., Cary, N.C.).

In cis enhancement of HCV entry by L-SIGN and DC-SIGN A panel of target cells, including hepatic, B-, T- and endothelial cell lines, are transiently transfected with constructs expressing L-SIGN or DC-SIGN, as well as mock transfected cells. Cell type-appropriate transfection protocols are applied in order to achieve the highest transfection efficiency in different cell lines. L-SIGN and DC-SIGN expression are analyzed by flow cytometry after labeling with specific antibodies.

Transfected cells are pulsed with serial ten-fold dilutions of HCV pseudovirion supernatants, standardized for E2 content. Pseudovirion entry is evaluated two days later by in situ histochemical staining for β-galactosidase activity and scoring of blue cells. Envelope glycoproteins from master quasispecies in patient sera, PBMCs and livers are incorporated into pseudovirions and tested in these experiments. Specificity controls are included to ascertain that increases in apparent viral titers are due to capture of pseudovirions by L-SIGN and DC-SIGN.

Assessing the Abilities of Different L-SIGN Alleles and Genotypes to Capture and Transmit HCV Pseudovirions to Target Cells

The efficiency with which different L-SIGN alleles and genotypes capture and transmit HCV pseudovirions to target cells is investigated. The fifteen human genotypes described by Liu et al. (2003) are first expressed in HCV-resistant cells, with each allele in a genotypic pair carrying either a C-terminal HA or His6 tag. Coding sequences for all seven L-SIGN alleles (Liu et al., 2003) found in humans are generated by PCR-based insertional and deletional mutagenesis and cloned into the plasmid, pcDNA3.1 (Invitrogen). Cells stably expressing L-SIGN alleles 3 through 9 are established by transfection and selection in G418. Furthermore, 3T3 and L1.2 cell lines are established expressing all fifteen genotypes identified by Liu et al., 2003. The bicistronic HIV-1-based vector, pLenti6 (Invitrogen), is engineered to express two alleles of L-SIGN. One allele is placed under the transcriptional control of the cytomegalovirus (CMV) promoter and is also appended with a C-terminal HA tag. The other allele is cloned under the transcriptional control of the simian virus 40 (SV40) promoter and is modified to carry a C-terminal His6 tag. VSV G (Lenti6-L-SIGN_{HA}-L-SIGN_{His6}) pseudovirions are generated and used to infect L1.2 cells or other HCV-resistant cell types. L-SIGN-expressing populations are isolated by cell sorting and subcloned by limiting dilution. RT-PCR is used to verify that both alleles are being expressed.

Similar levels of expression of both L-SIGN alleles are confirmed by western blotting with anti-HA and anti-His6 antibodies, though it is not known whether this reflects in vivo expression patterns as certain L-SIGN alleles may be dominant or recessive. It is also not clear whether different forms of L-SIGN form homo- or hetero-oligomers in the plasma membrane.

To determine if the coexpression of different alleles in this recombinant expression system results in L-SIGN hetero-oligomerization, cell extracts are immunoprecipitated with an anti-HA mAb and analyzed by western blotting using both anti-His6 and anti-HA antibodies. The coimmunoprecipitation of both His6-tagged L-SIGN and HA-tagged L-SIGN by the anti-HA mAb suggests that allelic L-SIGN variants form hetero-oligomers. Conversely, the immunoprecipitation of only HA-tagged L-SIGN molecules suggests that, despite coexisting in the same membrane, different L-SIGN forms do not interact with each other.

L-SIGN homo- and heterocomplexes are further characterized by BN-PAGE analysis which has been successfully used to investigate the oligomeric state of recombinant HIV envelope glycoproteins and other proteins (Schulke et al., 2002). Determining whether different forms of L-SIGN form hetero- or homo-oligomers provides insights into functional differences regarding their ability to capture and transmit HCV pseudovirions.

The ability of cells expressing different L-SIGN alleles and genotypes to bind HCV virions from patient sera, as well as their ability to promote binding of HCV pseudovirions comprising the envelope glycoproteins of master quasispecies from serum, PBMC and liver, is also ascertained (see L-SIGN and DC-SIGN capture of HCV pseudovirions comprising envelope glycoprotein of primary isolates). Finally, different L-SIGN+ clones are pulsed with serial 10-fold dilutions of HCV pseudovirion suspensions, washed and incubated with a panel of target cell lines as well as primary hepatocytes, B-lymphocytes, T-lymphocytes and macrophages in order to determine if there are differences in the efficiency with which they transmit HCV to target cells, as described above (see HCV pseudovirion entry into target cells mediated in trans by L-SIGN and DC-SIGN). The L-SIGN genotypes of HCV+ serum, PBMC and liver donors are ascertained in order to analyze any correlation between the presence of specific alleles and E1/E2 behavior in the capture and entry assays. Specificity controls in all experiments include inhibition by mannan and mAbs to the lectin domains as well as use of VSV and HIV-1 pseudotypes. The correlation of a particular genotype with increased or decreased transmission of HCV to target cells identifies particular alleles as resistance or susceptibility factors to HCV infection (cf. Liu et al., 2003).

Role of L-SIGN polymorphism in HCV transmission and disease In order to demonstrate an in vivo effect of L-SIGN polymorphism on HCV transmission and pathogenesis, L-SIGN haplotypes and genotypes in the HCV+ (resolved vs. persistent infection) and HCV− populations are identified and statistically analyzed. An initial patient cohort including approximately 100 HCV antibody-positive/RNA-negative, 100 HCV antibody-positive/RNA-positive, and 100 HCV antibody-negative/RNA-negative individuals is established. Persistently infected individuals are categorized according to disease stage and viral load.

Whole blood samples (5-10 ml) are collected from all individuals in the cohort and used to purify PBMC. Genomic DNA is prepared by standard lysis methods, and subjected to PCR amplification using primers 5′-TGTCCAAGGTCCCCAGCTCCC-3′ (SEQ ID NO:12) and 5′-GAACTCACCAAATGCAGTCTTCAAATC-3′(SEQ ID NO:13) specific for Exon 4 of L-SIGN (Bashirova et al., 2001). Typical PCR cycle conditions are: 94° C. for 5 s and 68° C. for 1 min. Alleles are identifed by 3% agarose gel electrophoresis and ethidium bromide staining, or other sizing techniques. Heterozygotes generate two distinct. bands of different sizes, whereas homozygotes generate a single band. Comparison to size markers allows the identification of alleles in individual donors.

The frequency of haplotypes and genotypes in the three designated populations are analyzed. The association between a specific genotype or haplotype and the risk of acquiring HCV are evaluated using Fisher's exact test, adjusted for multiple comparisons. Analyses between groups are performed using the log-rank test. A P value <0.05 is considered significant. The association between disease course and L-SIGN genotype and haplotype is similarly evaluated.

Results and Discussion

Anti-E2 Monoclonal Antibodies

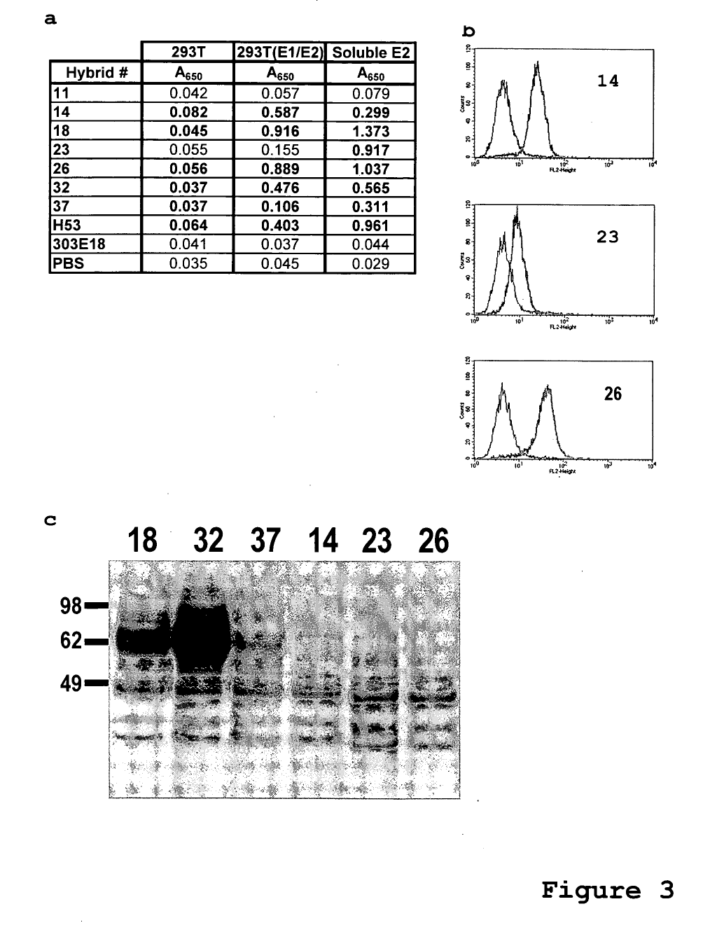

MAbs are valuable tools for studying the specificity of the interaction between viral envelope glycoproteins and putative cell surface receptors. In the present invention, mabs against cell surface-expressed HCV envelope glycoproteins were produced. This was accomplished by first immunizing mice with 293T cells transiently transfected with an E1-E2-expressing plasmid. Reactivity of the sera with the antigen was confirmed by ELISA. Hybridomas were generated and six hybridomas, designated 14, 18, 23, 26, 32 and 37, were found to secrete antibodies reactive with solubilized E1/E2 proteins as well as soluble E2 (FIG. 3a). No hybridomas tested thus far were reactive with E1, judging by reactivity with lysates of 293T cells expressing unmodified E1 alone (data not shown). Hybridoma supernatants were also tested by flow cytometry for their reactivity with E2 on HeLa cells stably expressing E1*-E2* from which putative splice acceptor sites have been deleted (FIG. 3b). Hybridoma supernatants 14, 23 and 26, 32 and 37 were reactive with E1/E2-expressing cells but not with parental HeLa cells. Furthermore, hybridoma supernatants 18, 32 and 37 were reactive with E2 after SDS-PAGE and transfer to nitrocellulose membranes, suggesting that these mAbs recognize linear epitopes (FIG. 3c).

Production of HCV Pseudovirions

Retroviral particles bud at the cell surface and non-specifically incorporate plasma membrane proteins into their envelopes. This feature can be exploited in order to pseudotype retroviral nucleocapsids with envelope glycoproteins of other viruses. Upon entry mediated by heterolbgous envelope glycoproteins, pseudovirions can deliver reporter genes and/or drug resistance genes into target cells.

Using the procedure of Bartosch et al. (2003b), it was demonstrated that E1/E2 mediated entry of pseudovirions into primary human hepatocytes as well as several hepatic and non-hepatic cell lines of human origin. Antibodies against E2 as well as HCV+ sera inhibited E1/E2-mediated entry. An anti-CD81 mAb (JS-81), as well as soluble CD81 ECL2 peptide, inhibited HCV pseudovirion entry, seemingly consistent with the hypothesis that CD81 serves as a receptor that mediates HCV entry into human cells (Jones et al., 2000). However, a number of CD81+ human cells of different lineages, including Jurkat, MOLT-4, CEM, Raji, TE671, HOS, HCT116 and A431 cells, did not support pseudovirion entry. Also, CD81 expression on murine CD81−, HCV-resistant cells did not render them permissive, suggesting the existence of an additional factor(s) mediating HCV entry into target cells.

Capture of HCV Pseudovirions by L-SIGN and DC-SIGN

To assess how L-SIGN- and DC-SIGN-mediated capture of HCV pseudovirions is modulated by differences in envelope glycoproteins, studies were initially conducted with HeLa cells modified to express L-SIGN and DC-SIGN (Gardner et al., 2003). However, these cells may not be ideal because they express CD81. Though no binding of soluble E2 or HCV virions to parental HeLa was detected under the assay conditions used, the presence of CD81 may have subtle and unwarranted effects, particularly in entry assays.

For this reason, murine 3T3 fibroblasts (adherent) and murine L1.2 T cell hybridomas (non-adherent) are engineered to express L-SIGN and DC-SIGN. It is necessary to ascertain that these transfected cells behave similarly to HeLa derivatives in virus capture assays. In subsequent studies, SIGN-expressing cells that are best suited for individual experiments are used; specifically, certain cocultures are more practical with non-adherent than adherent cell lines and vice versa. A valid L-SIGN and DC-SIGN virus-capture model is considered to have been developed if similar binding patterns and fold-differences are observed between matching pairs of HCV virions and pseudovirions.

Having optimized and validated the HCV pseudovirus-binding assay, L-SIGN- and DC-SIGN-mediated capture of HCV pseudovirions coated with E1/E2 of primary isolates is then examined. The ability of mannan and MAbs specific for the lectin-binding domains of L-SIGN and DC-SIGN to inhibit capture of HCV pseudotypes is systematically tested in order to ascertain the specificity of observed binding. It has been shown that E2 binding to immature MDDC is more DC-SIGN-dependent (Pohlmann et al., 2003), so HCV pseudovirion binding to both immature and mature MDDC is tested.

These experiments evaluate the ability of L-SIGN and DC-SIGN to capture HCV virions and HCV pseudovirions representing envelope glycoproteins from different host compartments. The ability of SIGN molecules to capture and transmit HCV to target cells indicates that these molecules play a significant role in viral replication in the host. A finding that entry receptors are ubiquitous suggests that L-SIGN and DC-SIGN may serve as the sole determinants of HCV tropism for lymphocytes and hepatocytes.

Entry of HCV Pseudovirions into Target Cells Mediated in trans by SIGN Molecules

Experiments are performed to assess the ability of L-SIGN and DC-SIGN to mediate in trans entry of HCV pseudovirions into target cells. In these experiments, HIV-1-based lacZ-expressing virions are pseudotyped with E1/E2. A variety of cell types, including transfectants expressing L-SIGN or DC-SIGN and nontransfected parental cells, are pulsed with HCV pseudovirions, cocultured with both permissive and non-permissive cell lines, and stained for β-galactosidase activity (blue coloration).

The detection of blue cells in cocultures with MDDC, L-SIGN+ and DC-SIGN+ cells but not parental cells, is interpreted to mean that SIGN molecules can transmit infectious virus to permissive target cells. The detection of blue cells after capture of suboptimal viral concentrations implies that infection of target cells is enhanced.

Entry of HCV Pseudovirions into Target Cells Enhanced in cis by SIGN Molecules

Enhancement of HCV pseudovirion entry in cis probably has little physiological relevance since HCV target cells are unlikely to express SIGN molecules on their surface. However, observing in cis enhancement of infection would validate using L-SIGN or DC-SIGN expressing target cells to, for example, generate cDNA expression libraries that could be used for functional cloning of viral receptors. In order to demonstrate that SIGN molecules enhance in cis infection, a panel of target cells are transiently transfected with constructs expressing L-SIGN or DC-SIGN, as well as mock transfected cells. Transfected cells expressing SIGN molecules are pulsed with HCV pseudovirions, and pseudovirion entry is evaluated by scoring of blue cells in a blue cell (β-galactosidase) assay. An increase in apparent viral titers, evidenced by higher numbers of blue cells, indicates that pseudovirion entry is enhanced by the expression of the SIGN molecules which capture the pseudovirions.

L-SIGN Isoforms are Functionally Expressed at the Cell Surface

Liu et al. (2003) have determined that the L-SIGN repeat region exists in seven allelic forms, with the number of repeats ranging from three to nine. Combinations of these seven alleles have been shown to constitute 15 genotypes: 4/4, 5/3, 5/4, 5/5, 6/4, 6/5, 6/6, 7/4, 7/5, 7/6, 7/7, 8/6, 8/7, 9/5 and 9/7 (Liu et al., 2003). In the present study, the functional expression of different L-SIGN alleles in mammalian cells was investigated. The efficiency with which the proteins encoded by these different L-SIGN alleles capture and transmit HCV pseudovirions to target cells was also investigated.

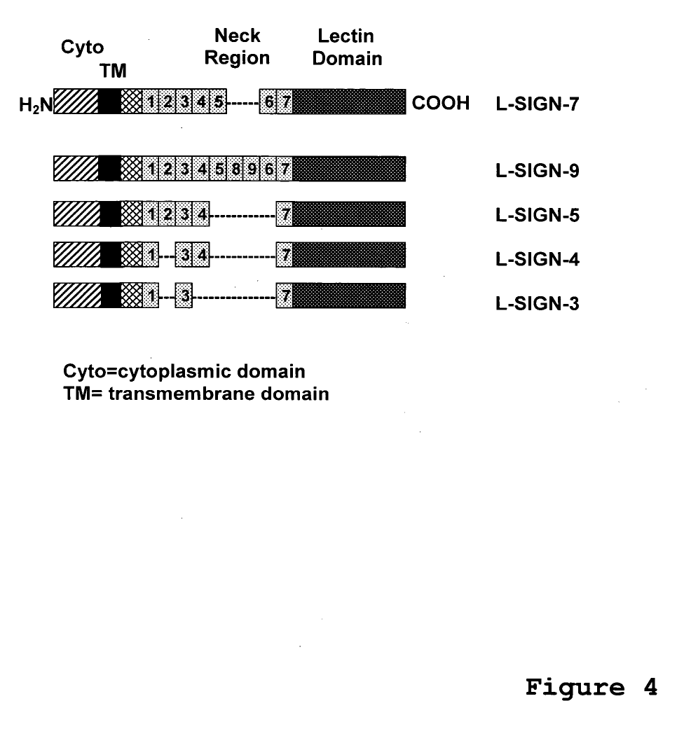

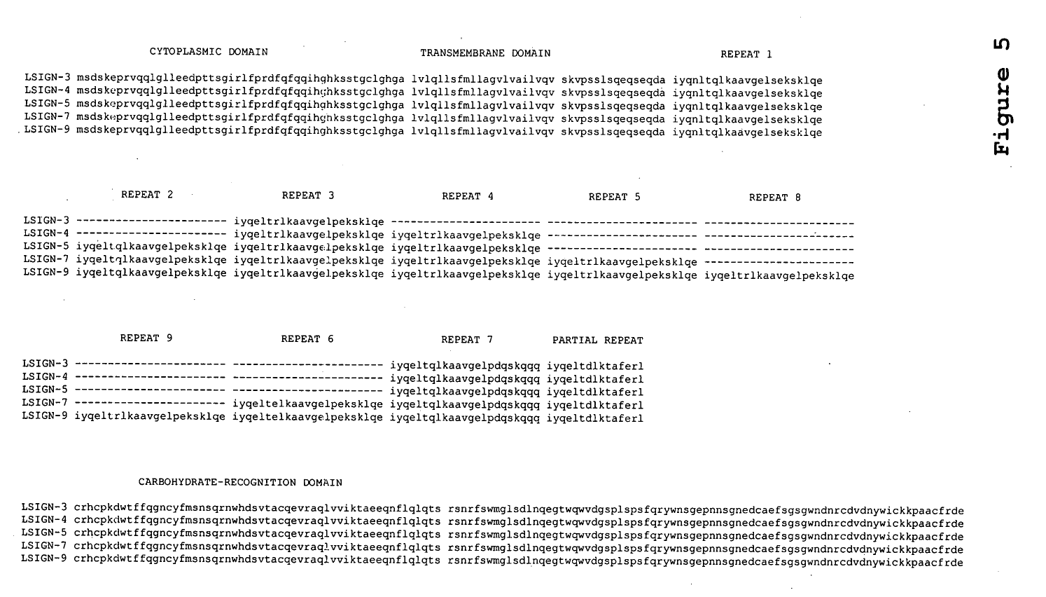

FIG. 4 depicts the domain structures of the proteins encoded by five L-SIGN isoforms examined in this study. The 23-amino-acid repeat segments are numbered relative to those encoded by L-SIGN-7, the most common form (Bashirova et al., 2001), with the first repeat beginning at Ile-89, as previously described (Mummidi et al., 2001). Ile-89 represents position d of the heptad repeat. L-SIGN-3 encodes tandem repeats 1, 3 and 7; L-SIGN-4 encodes repeats 1, 3, 4 and 7; L-SIGN-5 encodes repeats 1-4 and 7; L-SIGN-6 encodes repeats 1-4, 6 and 7; and L-SIGN-9 has a 2-repeat insertion between repeats 5 and 6 of L-SIGN-7. In addition, all alleles encode the partial (15 amino acid) repeat sequence located carboxy-terminal to repeat 7 (not shown). The deduced protein sequences of the L-SIGN isoforms are shown in FIG. 5.

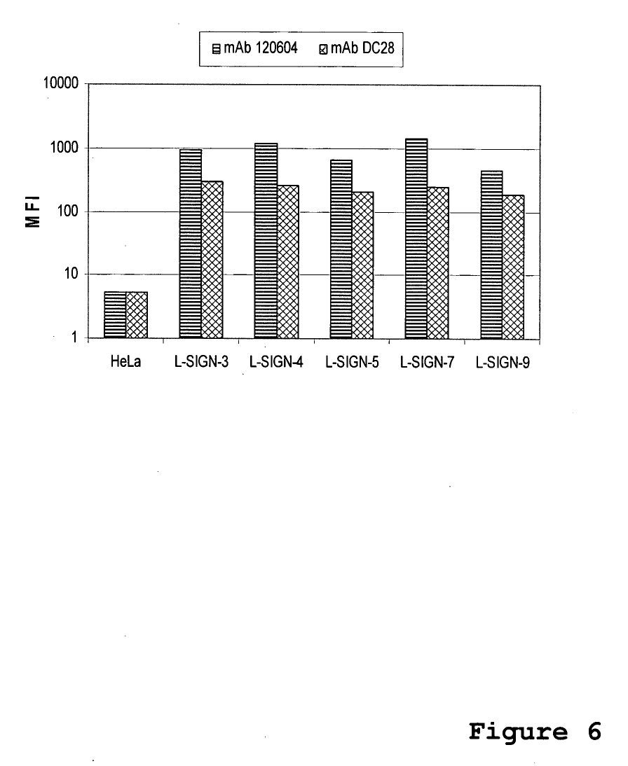

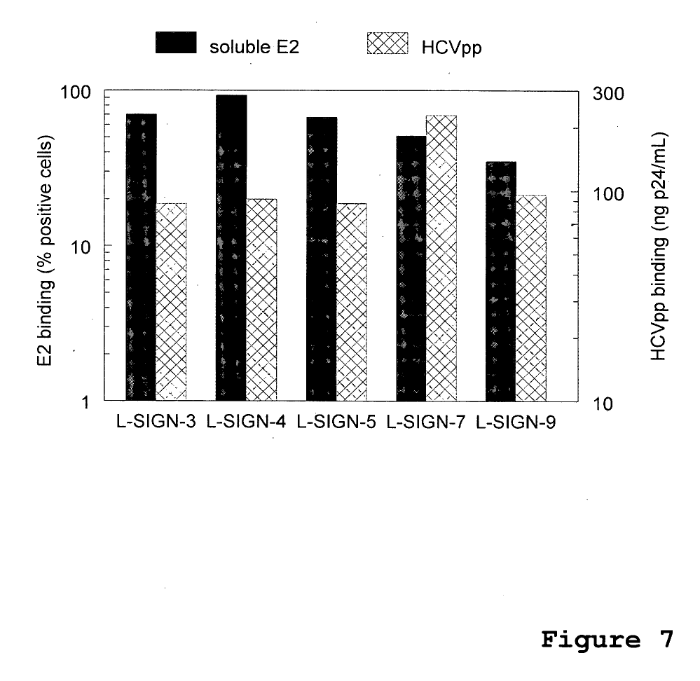

HeLa cells were modified to stably express L-SIGN isoforms containing 3, 4, 5, 7, and 9 tandem repeats. No studies examining expression of variant L-SIGN isoforms on the cell surface have previously been reported, and the present study describes the first evaluation of the expression and function of L-SIGN isoforms in mammalian cells. The transfectants were first analyzed by flow cytometry using mabs to the CRD and neck region of L-SIGN. Mean fluorescence intensity (MFI) values for the different isoforms are plotted in FIG. 6 to facilitate comparison with the binding data for E2 and HCVpp which are shown in FIG. 7.

As illustrated in FIG. 6, each of the isoforms was detected at high levels on the cell surface, demonstrating that alleles encoding 3, 4, 5, 7 and 9 tandem repeats are efficiently expressed, and that the translated proteins are transported to the plasma membrane of HeLa cells.

Each of the isoforms showed similar levels of binding to a mAb (DC28) that is specific to the repeat region. Therefore, the epitope for this mAb is accessible when the both the longer and shorter isoforms are expressed on the cell surface. Using a mAb (120604) specific for the CRD, comparable levels of binding were observed for L-SIGN-3, L-SIGN-4 and L-SIGN-7, whereas binding to L-SIGN-5 and L-SIGN-9 was approximately 2- and 3-fold lower, respectively (FIG. 6). Similar results were obtained using another CRD-specific mAb (120612, data not shown). Whereas mAb DC28 binds a linear epitope, mAbs 120604 and 120612 recognize conformational epitopes, indicating that the repeat-region mutations do not disrupt the conformation of the CRD. Background levels of binding were observed using an isotype-control mAb and the DC-SIGN-specific mAb 120507 (data not shown).

L-SIGN Isoforms Bind HCV E2 and HCVpp

It was previously demonstrated that DC-SIGN and L-SIGN-7 specifically bind HCV E2, HCVpp and naturally occurring HCV virions (Gardner et al. 2003; Cormier et al., 2004b). In the present study, flow cytometry was used to examine binding of E2 and HCVpp to L-SIGN isoforms. Soluble E2 glycoprotein bound all isoforms tested (FIG. 7). Levels of specific binding to HeLa-SIGN transfectants ranged from 35% to 93% of cells, or 9- to 25-fold above background binding to parental HeLa (3.8%). The pattern of E2 binding was similar to that observed using the anti-CRD mAb (FIG. 6), with L-SIGN-9 cells showing the lowest levels of binding in each case. The anti-CRD mAb and E2 data indicate that L-SIGN-9 cells may have 2- to 3-fold lower levels of expression or presentation of the CRD.

HCVpp also specifically bound HeLa cells expressing each of the L-SIGN isoforms (FIG. 7). Similar levels of binding were observed for the various isoforms with the exception of L-SIGN-7, for which binding was approximately 2-fold higher. HCVpp binding was unaffected by incubation of cells with JS-81 (10 μg/ml) for 30 min prior to addition of viral supernatants, indicating that binding is not mediated by CD81 (data not shown).

Based on the data in FIG. 7, the relative binding of E2 and HCVpp to each isoform was compared. By comparison to L-SIGN-7, the shorter isoforms (L-SIGN-3, -4, and -5) exhibited a modest preference for binding soluble E2 over HCVpp; however, the overall patterns of binding were similar for each of the variant isoforms tested.

L-SIGN Isoforms Vary in their Abilities to Mediate Trans-Infection.

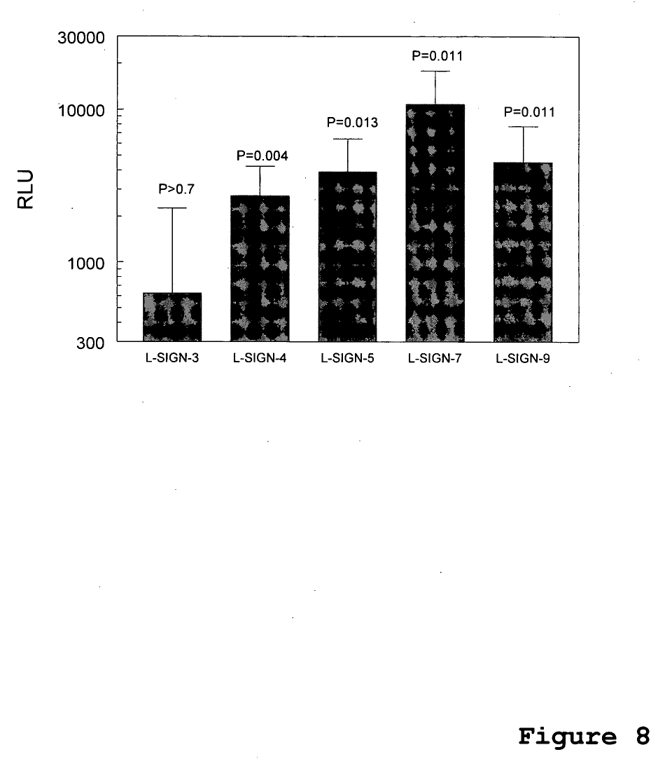

Parental HeLa and HeLa-SIGN transfectants were analyzed for their abilities to mediate trans-infection of Hep3B cells by HCVpp. Compared to parental HeLa, L-SIGN-4 (P=0.004), L-SIGN-5 (P=0.013), L-SIGN-7 (P=0.011) and L-SIGN-9 (P=0.011) transfectants all mediated statistically significant levels of trans-infection (FIG. 8). In contrast, L-SIGN-3 transfectants did not mediate trans-infection (P >0.7) despite high-level expression (FIG. 6) and binding of E2 and HCVpp (FIG. 7). In the present system, 4 repeat segments therefore represent the minimum requirement for efficient trans-infection.

The highest levels of trans-infection were observed for L-SIGN-7 cells, followed by L-SIGN-9, -5, -4 and -3 (inactive). The differences between L-SIGN-7 and the other isoforms were statistically significant in 2-sided t-tests for L-SIGN-3 (P=0.011), L-SIGN-4 (P=0.033) and L-SIGN-5 (P=0.049). The data for L-SIGN-9 were significant in a 1-sided (P=0.045) but not 2-sided (P=0.090) test. L-SIGN-9 mediated high levels of trans-infection despite 2- to 3-fold lower levels of binding to anti-CRD mAb (FIG. 6) and E2 (FIG. 7) than the other isoforms.

The discordance between HCVpp trans-infection and binding was unexpected. However, this observation reaffirms prior findings that virus binding to L-SIGN-7 and DC-SIGN is necessary but not sufficient for trans-infection (Cormier et al., 2004b; Kwon et al., 2002; Pohlmann et al., 2001; Baribaud et al., 2001; Wu et al., 2004). Several factors could contribute to the inter-allelic differences in the trans-infectivity of bound HCVpp. First, for example, shortened neck regions could sub-optimally present virus to the target cell for simple steric reasons, i.e., the virus may be bound too close to the surface of the SIGN-expressing cell membrane for optimal presentation to target cells.

Second, allelic variation in trans-infection could reflect differences in the oligomeric states of the L-SIGN isoforms. Support for this notion is provided by a recent study by Feinberg et al. (2004). Cell-surface L-SIGN-7 and DC-SIGN exist as tetramers, as do recombinant, soluble forms of these proteins (Feinberg et al., 2004; Mitchell et al., 2001). However, soluble SIGN molecules containing 2 tandem repeats formed mixtures of monomers and dimers, whereas a 5-repeat version of soluble DC-SIGN formed a mixture of dimers and tetramers (Feinberg et al., 2004). Although L-SIGN-3 was not examined in the prior study, the findings imply that this isoform is unlikely to form stable tetramers, at least in the context of soluble protein.

Third, endocytosis and recycling of SIGN-bound virus are critical for trans-infection (Cormier et al., 2004b; Kwon et al., 2002; Pohlmann et al., 2001), and the results of the present study could reflect allelic differences in the extent or pathway of receptor internalization. Unlike other ligands (Engering et al., 2002), pathogens bound to L-SIGN-7 and DC-SIGN are routed into early endosomes (Kwon et al., 2002; Ludwig et al., 2004), where they are protected from degradation. The mildly acidic (pH 6-6.8) environment of early endosomes (Mellman, 1996) is critical for trans-infection, as neutralization with lysosomotropic agents inhibits trans-infection (Cormier et al., 2004b; Kwon et al., 2002). Lack of trans-infection by L-SIGN-3 could reflect reduced levels of receptor internalization or its preferential routing of HCVpp to late endosomal/lysosomal compartments for degradation.

Trans-Infection is Blocked by Agents that Bind the CRD of L-SIGN

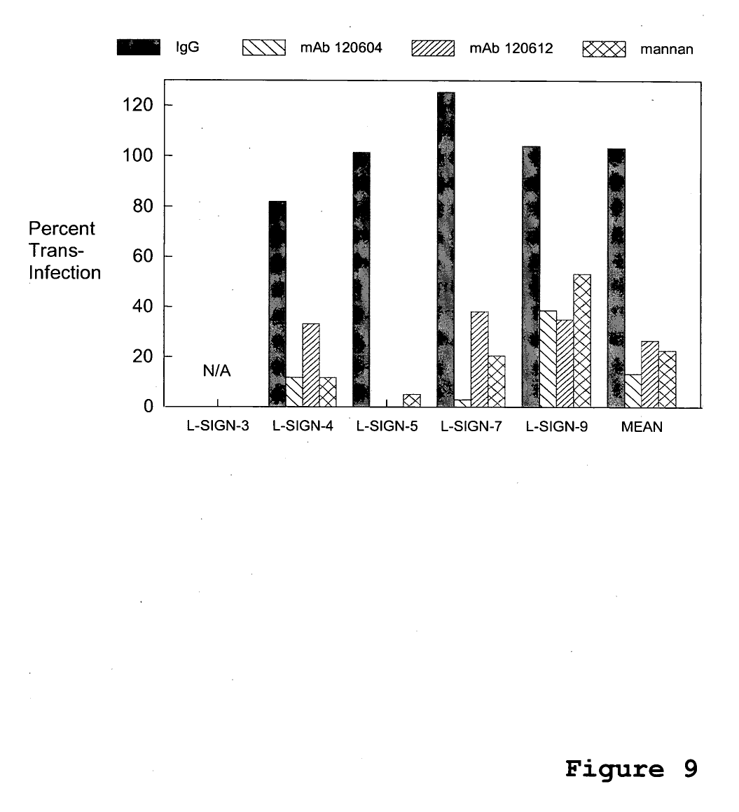

Trans-infection was also examined in the presence of agents that bind the CRD of L-SIGN-7 and inhibit binding of HCVpp (Cormier et al. 2004b). Mannan (20 μg/ml), mAb 120604 (10 μg/ml) and mAb 120612 (10 μg/ml) efficiently blocked trans-infection mediated by each of the functional L-SIGN alleles: L-SIGN-4, -5, -7 and -9 (FIG. 9). The level of trans-infection was inhibited by 62-100% for mAb 120604, 62-100% for mAb 120602, and 47-95% for mannan. In contrast, isotype-control mouse IgG had no effect on trans-infection (percent inhibition <18% of control for all isoforms). There was no obvious variation between the different isoforms in the potency of the inhibitors, and the average level of inhibition across the different isoforms was 87% for mAb 120604, 73% for mAb 120612, 77% for mannan and <5% for isotype-control IgG (FIG. 9). The data indicate that L-SIGN-4, -5, and -9 mediate trans-infection via interactions between their CRD and HCVpp, as previously observed for L-SIGN-7 (Cormier et al. 2004b; Lozach et al., 2004). No significant trans-infection was observed for L-SIGN-3 in the presence or absence of inhibitors (data not shown).

Effect of L-SIGN Polymorphisms Transmission and Pathogenesis of HCV and Other Pathogens

The present study is the first to evaluate the expression and function of L-SIGN isoforms in mammalian cells. It has been demonstrated that L-SIGN transcripts encoding 3, 4, 5, 7 and 9 tandem repeats, respectively, are efficiently translated in HeLa cells, the protein isoforms are exported to the surface of HeLa cells, and are reactive with mAbs to the CRD and repeat region. It was further demonstrated that each of these isoforms bound similar levels of both HCV E2 and HCVpp. However, significant allelic differences were observed in trans-infection of liver cells by HCVpp. The most common form of L-SIGN (L-SIGN-7) mediated the highest levels of trans-infection, and the efficiency of trans-infection decreased for isoforms with progressive deletions of tandem repeats. These differences in trans-infection were not reflected in corresponding decreases in receptor expression or E2 binding. Thus, no trans-infection was observed for L-SIGN-3 despite high levels of cell-surface expression and binding to E2 and HCVpp. These findings suggest that L-SIGN-7 presents a preferred configuration for trans-infection.

The data presented herein may have implications for the transmission and pathogenesis of HCV. As previously noted, L-SIGN-7 may capture HCV in the liver and deliver virus to susceptible hepatocytes, thereby facilitating establishment and dissemination of infection (Gardner et al. 2003; Pohlmann et al., 2003; Lozach et al., 2003; 2004). The present study demonstrates that L-SIGN-4, -5 and -9 also mediated trans-infection, albeit with varying efficiencies, whereas L-SIGN-3 bound HCVpp without facilitating trans-infection. These findings support a molecular mechanism whereby genetic polymorphisms in L-SIGN and DC-SIGN could impact diseases mediated by HCV and other pathogens recognized by L-SIGN and DC-SIGN, and in particular, could afford protection against HCV infection and disease progression. For example, the expression of the L-SIGN-3 polypeptide, which has been demonstrated herein not to mediate trans-infection by HCVpp, may help confer on a subject some level of resistance against infection by HCV and other pathogens that bind to L-SIGN. Expression of L-SIGN-3 may be more prevalent in high-risk individuals who remain uninfected by HCV or in individuals who resolve disease. The L-SIGN-4 and -5 polypeptides may also confer some level of resistance. Conversely, expression of L-SIGN-7, as well as L-SIGN-9, may enhance susceptibility to infection by pathogens such as HCV that bind to L-SIGN. In addition, polymorphisms in L-SIGN and DC-SIGN could affect HCV disease by modulating host immune responses to the virus.

Similarly, direct and trans-infection of other pathogens may be affected by L-SIGN polymorphisms. Collectively, L-SIGN and DC-SIGN have been demonstrated to bind or facilitate infection by a diverse array of viral and non-viral pathogens, including HIV-1 and other primate lentiviruses (Geijtenbeek et al., 2000; Baribaud et al., 2001; Lee et al., 2001), Ebola virus (Alvarez et al., 2002), Marburg virus (Marzi et al., 2004), dengue virus (Tassaneetrithep et al., 2003), SARS coronavirus (Marzi et. al., 2004; Yang et al., 2004), cytomegalovirus (Halary et al., 2002), Sindbis virus (Klimstra et al., 2003), Leishmania amastigotes (Colmenares et al., 2002), Mycobacterium tuberculosis (Geijtenbeek et al., 2003), Candida albicans (Cambi et al., 2003), Helicobacter pylori (Bergman et al., 2004), and Aspergillus fumigatus (Serrano-Gomez et al., 2004). However, the specificity of the interaction is indicated by the observation that several viruses are not recognized by the SIGN molecules (Halary et al., 2002; Hong et al., 2002), and recognition of other viruses and pathogens is limited to certain stages of their life cycles (Klimstra et al., 2003; Colmenares et al., 2002; Bergman et al., 2004). HIV-1 provides the most well-studied example with many parallels to HCV, and the data reported herein suggest that repeat-region polymorphisms in L-SIGN are likely to influence HIV-1 trans-infection.

Recent studies have demonstrated that genetic polymorphisms in DC-SIGN affect HIV-1 transmission (Liu et al., 2004; Martin et al., 2004). In contrast to L-SIGN, repeat-region polymorphisms in DC-SIGN are rare, but a 6-repeat form of DC-SIGN (DC-SIGN-6) was shown to confer protection against mucosal infection by HIV-1 (Liu et al., 2004). This finding is consistent with the generally held model whereby DC-SIGN-expressing dendritic cells bind HIV-1 at mucosal sites of exposure, transport the virus to the draining lymph nodes, and mediate trans-infection of susceptible lymphocytes (Geijtenbeek et al., 2000). In a second study, a promoter-region polymorphism (−336C) in DC-SIGN was associated with an increased risk of parenteral but not mucosal infection by HIV-1 (Martin et al., 2004). This result runs contrary to the model described above, and the findings underscore the complexity of the interactions between pathogens and C-type lectins. Neither Liu et al. (2004) nor Martin et al. (2004) examined the molecular mechanism whereby DC-SIGN polymorphisms confer protection against HIV-1 infection.

In summary, the data presented herein demonstrate that naturally-occurring repeat-region isoforms of L-SIGN are efficiently translated and expressed at the surface of mammalian cells. Each isoform was recognized by conformation-specific mAbs and specifically bound HCV E2 and HCVpp. However, the efficiency of trans-infection of liver cells decreased with progressive deletions of repeat segments, such that the shortest isoform (L-SIGN-3) was inactive in mediating HCVpp trans-infection. These findings support a molecular mechanism whereby genetic polymorphisms could impact diseases mediated by HCV and other pathogens recognized by L-SIGN and DC-SIGN.

Based on the data presented hereinabove, the determination of L-SIGN genotypes for individual patients enables the prediction of susceptibility or resistance to HCV and other pathogens, disease sequelae and potential treatment options. Such information on the L-SIGN genotype status of a patient is valuable to the clinician and for development of L-SIGN variant-specific drugs for patient-specific treatment. Novel agents, including peptidyl agents, antibodies, non-peptidyl agents and small molecules, that bind preferentially to particular L-SIGN polypeptides are identified using screening methods described herein. Certain of these agents, which bind to L-SIGN polypeptides associated with disease susceptibility, inhibit binding of HCV and other pathogens and thus are used, for example, to inhibit pathogen infection of a subject or to treat a subject afflicted with a pathogen-associated disorder. Such agents also have wide application in deriving patient-specific therapies. In addition, these agents, as well as agents, including antibodies, that conversely bind to non-disease-associated L-SIGN variants, provide valuable diagnostic tools that are used in genetic screening of a subject for susceptibility or resistance to pathogen infection.

### REFERENCES

U.S. Pat. No. 4,816,567 issued to Cabilly et al. on Mar. 28, 1989.

U.S. Pat. No. 5,225,539 issued to Winter on Jul. 6, 1993.

U.S. Pat. No. 5,545,806 issued to Lonberg et al. on Aug. 13, 1996.

U.S. Pat. No. 5,545,807 issued to Surani et al. on Aug. 13, 1996.

U.S. Pat. No. 5,585,089 issued to Queen et al. on Dec. 17, 1996.

U.S. Pat. No. 5,591,669 issued to Krimpenfort et al. on Jan. 7, 1997.

U.S. Pat. No. 5,598,369 issued to Chen et al. on Jan. 28, 1997.

U.S. Pat. No. 5,693,761 issued to Queen et al. on Dec. 2, 1997.

U.S. Pat. No. 5,882,852 issued to Bukh et al. on Mar. 16, 1999.

U.S. Pat. No. 6,150,584 issued to Kucherlapati et al. on Nov. 21, 2000.

U.S. Pat. No. 6,572,864 issued to Bukh et al. on Jun. 3, 2003.

U.S. Ser. No. 10/184,150, filed Jun. 26, 2002.

U.S. Ser. No. 10/328,997, filed Dec. 24, 2002.

PCT International Application No. PCT/US89/05857, filed Dec. 28, 1989, International Publication No. WO 90/07861, published Jul. 26, 1990.

European Patent Application No. EP 1046651 A1, published Oct. 25, 2000.

European Patent Application No. EP 1086137 A1, published Mar. 28, 2001.

Afonso, A. M., J. Jiang, F. Penin, C. Tareau, D. Samuel, M. A. Petit, H. Bismuth, E. Dussaix and C. Feray (1999) Nonrandom distribution of hepatitis C virus quasispecies in plasma and peripheral blood mononuclear cell subsets. J. Virol. 73: 9213-21.

Alter, H. J. and L. B. Seef (1993) Transfusion-associated hepatitis. In “Viral Hepatitis” (Z. A. Thomas, Ed.). Churchill Livingstone, Edinburgh.

Alvarez, C. P., F. Lasala, J. Carrillo, O. Muniz, A. L. Corbi and R. Delgado (2002) C-type lectins DC-SIGN and L-SIGN mediate cellular entry by Ebola virus in cis and in trans. J. Virol. 76: 6841-6844.

Anonymous (1999) Global surveillance and control of hepatitis C. Report of a WHO Consultation organized in collaboration with the Viral Hepatitis Prevention Board, Antwerp, Belgium. J. Viral. Hepat. 6: 35-47.

Baribaud, F., S. Pohlmann, G. Leslie, F. Mortari and R. W. Doms (2002) Quantitative expression and virus transmission analysis of DC-SIGN on monocyte-derived dendritic cells. J. Virol. 76: 9135-9142.

Baribaud, F., S. Pohlmann, T. Sparwasser, M. T. Kimata, Y. K. Choi et al. (2001) Functional and antigenic characterization of human, rhesus macaque, pigtailed macaque, and murine DC-SIGN. J. Virol. 75: 10281-10289.

Barlow, K. L., J. H. Tosswill, J. V. Parry and J. P. Clewley (1997) Performance of the Amplicor human immunodeficiency virus type 1 PCR and analysis of specimens with false-negative results. J. Clin. Microbiol. 35: 2846-2853.

Bartosch, B., A. Vitelli, C. Granier, C. Goujon, J. Dubuisson, S. Pascale, E. Scarselli, R. Cortese, A. Nicosia and F. L. Cosset (2003a) J. Biol. Chem. 278: 41624-41630.

Bartosch, B., J. Dubuisson and F. L. Cosset (2003b) Infectious hepatitis C virus pseudo-particles containing functional E1-E2 envelope protein complexes. J. Exp. Med. 197: 633-642.

Bashirova, A. A., T. B. Geijtenbeek, G. C. van Duijnhoven, S. J. van Vliet, J. B. Eilering et al. (2001) A dendritic cell-specific intercellular adhesion molecule 3-grabbing nonintegrin (DC-SIGN)-related protein is highly expressed on human liver sinusoidal endothelial cells and promotes HIV-1 infection. J. Exp. Med. 193: 671-678.

Berger, A., L. Scherzed, M. Sturmer, W. Preiser, H. W. Doerr and H. F. Rabenau (2002) Evaluation of the Cobas AmpliPrep/Cobas Amplicor HIV-1 Monitor Ultrasensitive Test: comparison with the Cobas Amplicor HIV-1 Monitor test (manual specimen preparation). J. Clin. Virol. 25 Suppl. 3: S-103-107.

Bergman, M. P., A. Engering, H. H. Smits, S. J. van Vliet, A. A. van Bodegraven, H. P. Wirth, M. L. Kapsenberg, C. M. J. E. Vandenbroucke-Grauls, Y. van Kooyk and B. J. Appelmelk (2004) Helicobacter pylori modulates the T helper cell 1/T helper cell 2 balance through phase-variable interaction between lipopolysaccharide and DC-SIGN. J. Exp. Med. 200: 979-990.

Binley, J. M., R. W. Sanders, B. Clas, N. Schuelke, A. Master, Y. Guo, F. Kajumo, D. J. Anselma, P. J. Maddon, W. C. Olson and J. P. Moore (2000) A recombinant human immunodeficiency virus type 1 envelope glycoprotein complex stabilized by an intermolecular disulfide bond between the gp120 and gp41 subunits is an antigenic mimic of the trimeric virion-associated structure. J. Virol. 74: 627-643.

Boisvert, J., X. S. He, R. Cheung, E. B. Keeffe, T. Wright and H. B. Greenberg (2001) Quantitative analysis of hepatitis C virus in peripheral blood and liver: replication detected only in liver. J. Infect. Dis. 184: 827-835.

Botstein, D., R. L. White, M. Skolnick and R. W. Davis (1980) Construction of a genetic linkage map in man using restriction fragment length polymorphisms. Am. J. Hum. Genet. 32: 314-331.

Cambi, A., K. Gijzen, K., J. M. de Vries, R. Torensma, B. Joosten, G. J. Adema, M. G. Netea, B. J. Kullberg, L. Romani and C. G. Figdor (2003) The C-type lectin DC-SIGN (CD209) is an antigen-uptake receptor for Candida albicans on dendritic cells. Eur. J. Immunol. 33: 532-538.

Choy, Y. S., S. L. Dabora, F. Hall, V. Ramesh, Y. Niida et al. (1999) Superiority of denaturing high performance liquid chromatography over single-stranded conformation and conformation-sensitive gel electrophoresis for mutation detection in TSC2. Ann. Hum. Genet. 63: 383-391.

Colmenares, M., A. Puig-Kroger, P. O. Muniz, A. L. Corbi and L. Rivas (2002) Dendritic cell (DC)-specific intercellular adhesion molecule 3 (ICAM-3)-grabbing nonintegrin (DC-SIGN, CD209), a C-type surface lectin in human DCs, is a receptor for Leishmania amastigotes. J. Biol. Chem. 277: 36766-36769. Connor, R. I., B K. Chen, S. Choe and N. R. Landau (1995) Vpr is required for efficient replication of human immunodeficiency virus type-1 in mononuclear phagocytes. Virology 206: 935-944.

Cooper, S., A. L. Erickson, E. J. Adams, J. Kansopon, A. J. Weiner, D. Y. Chien, M. Houghton, P. Parham and C. M. Walker (1999) Analysis of a successful immune response against hepatitis C virus. Immunity 10: 439-449.

Cormier, E. G., F. Tsamis, F. Kajumo, R. J. Durso, J. P. Gardner, and T. Dragic (2004a) CD81 is an entry coreceptor for hepatitis C virus. Proc. Natl Acad. Sci. USA 101: 7270-7274.

Cormier, E. G., R. J. Durso, F. Tsamis, L. Boussemart, C. Manix, W. C. Olson, J. P. Gardner and T. Dragic (2004b) L-SIGN (CD209L) and DC-SIGN (CD209) mediate transinfection of liver cells by hepatitis C virus. Proc. Natl. Acad. Sci. USA 101: 14067-14072.

Doms, R. W. and J. P. Moore (2000) HIV-1 membrane fusion: targets of opportunity. J. Cell Biol. 151: F9-14.

Dubuisson, J., H. H. Hsu, R. C. Cheung, H. B. Greenberg, D. G. Russell and C. M. Rice (1994) Formation and intracellular localization of hepatitis C virus envelope glycoprotein complexes expressed by recombinant vaccinia and Sindbis viruses. J. Virol. 68: 6147-6160.

Dumonceaux, J., E. G. Cormier, F. Kajumo, G. P. Donovan, J. Roy-Chowdhury, I. J. Fox, J. P. Gardner, and T. Dragic, (2003) Expression of unmodified hepatitis C virus envelope glycoprotein-coding sequences leads to cryptic intron excision and cell surface expression of E1/E2 heterodimers comprising full-length and partially deleted E1. J. Virol. 77: 13418-13424.

Engering, A., T. B. Geijtenbeek, S. J. van Vliet, M. Wijers, E. van Liempt, N. Demaurex, A. Lanzavecchia, J. Fransen, C. G. Figdor, V. Piguet et al. (2002) The dendritic cell-specific adhesion receptor DC-SIGN internalizes antigen for presentation to T cells. J Immunol. 168: 2118-26.

Feinberg, H., Y. Guo, D. D. Mitchell, K. Drickamerand W. I. Weis (2004) Extended neck regions stabilize tetramers of the receptors DC-SIGN and DC-SIGNR. J. Biol. Chem. 280: 1327-1335.

Ferrini, J. B., L. Pichard, J. Domergue and P. Maurel (1997) Long-term primary cultures of adult human hepatocytes. Chem. Biol. Interact. 107: 31-45.

Flint, M., J. Dubuisson, C. Maidens, R. Harrop, G. R. Guile, P. Borrow and J. A. McKeating (2000) Functional characterization of intracellular and secreted forms of a truncated hepatitis C virus E2 glycoprotein. J. Virol. 74: 702-709.

Flint, M., J. M. Thomas, C. M. Maidens, C. Shotton, S. Levy, W. S. Barclay and J. A. McKeating (1999) Functional analysis of cell surface-expressed hepatitis C virus E2 glycoprotein. J. Virol. 73: 6782-6790.

Fournier, C., C. Sureau, J. Coste, J. Ducos, G. Pageaux, D. Larrey, J. Domergue and P. Maurel (1998) In vitro infection of adult normal human hepatocytes in primary culture by hepatitis C virus. J. Gen. Virol. 79: 2367-2374.

Fry, D. E. and L. M. Flint (1997) Hepatitis: an overview of important issues. Bull. Am. Coll. Surg. 82: 8-13.

Gardner, J., R. J. Durso, R. R. Arrigale, G. P. Donovan, P. J. Maddon, T. Dragic and W. C. Olson (2003) L-SIGN is a liver-specific capture receptor for hepatitis C virus. Proc. Natl. Acad. Sci. USA 100: 4498-4503.

Garry, R. F. and S. Dash (2003) Proteomics computational analyses suggest that hepatitis C virus E1and pestivirus E2 envelope glycoproteins are truncated class II fusion proteins. Virology 307: 255-265.

Geijtenbeek, T. B., D. S. Kwon, R. Torensma, S. J. van Vliet, G. C. van Duijnhoven, et al. (2000) DC-SIGN, a dendritic cell-specific HIV-1-binding protein that enhances trans-infection of T cells. Cell 100: 587-597.

Geijtenbeek, T. B., S. J. van Vliet, E. A. Koppel, M. Sanchez-Hernandez, C. M. J. E. Vandenbroucke-Grauls, B. Appelmelk and Y. van Kooyk (2003) Mycobacteria target DC-SIGN to suppress dendritic cell function. J. Exp. Med. 197: 7-17.

Gilljam, G. (1993) Envelope glycoproteins of HIV-1, HIV-2, and SIV purified with Galanthus nivalis agglutinin induce strong immune responses. AIDS Res. Hum. Retroviruses 9: 431-438.

Goldberg, M., L. Smith, II, N. Tamayo and A. S. Kiselyov (1999) Solid support synthesis of 14-macrocycles containing. 4-hydroxyproline structural unit via S_{N}Ar methodology. Tetrahedron 55: 13887-13898.

Grakoui, A., C. Wychowski, C. Lin, S. M. Feinstone and C. M. Rice (1993) Expression and identification of hepatitis C virus polyprotein cleavage products. J. Virol. 67: 1385-1395.

Gray, I. C., D. A. Campbell and N. K. Spurr (2000) Single nucleotide polymorphisms as tools in human genetics. Hum. Mol. Genet. 9: 2403-2408.

Halary, F., A. Amara, H. Lortat-Jacob, M. Messerle, T. Delaunay, C. Houles, F. Fieschi, F. Arenzana-Seisdedos, J. F. Moreau and J. Dechanet-Merville (2002) Human cytomegalovirus binding to DC-SIGN is required for dendritic cell infection and target cell trans-infection. Immunity 17: 653-664.

Higginbottom, A., E. R. Quinn, C. C. Kuo, M. Flint, L. H. Wilson et al. (2000) Identification of amino acid residues in CD81 critical for interaction with hepatitis C virus envelope glycoprotein E2. J. Virol. 74: 3642-3649.

Hong, P. W., K. B. Flummerfelt, A. De Parseval, K. Gurney, J. H. Elder and B. Lee (2002) Human immunodeficiency virus envelope (gp120) binding to DC-SIGN and primary dendritic cells is carbohydrate dependent but does not involve 2G12 or cyanovirin binding sites: implications for structural analyses of gp120-DC-SIGN binding. J. Virol. 76: 12855-12865.

Ikeda, M., K. Sugiyama, T. Mizutani, T. Tanaka, K. Tanaka, H. Sekihara, K. Shimotohno and N. Kato (1998) Human hepatocyte clonal cell lines that support persistent replication of hepatitis C virus. Virus Res. 56: 157-167.

Jameson, B, F. Baribaud, S. Pohlmann, D. Ghavimi, F. Mortari, R. W. Doms and A. Iwasaki (2002) Expression of DC-SIGN by dendritic cells of intestinal and genital mucosae in humans and rhesus macaques. J. Virol. 76: 1866-1875.

Jones, I. M., C. Chan-Fook, W. R. Jiang and B. E. Clarke (2000) Receptors for hepatitis C virus. J. Virol. 74: 10860-10861.

Kiselyov, A., S. Eisenberg and Y. Luo (1998) Solid support synthesis of 14-membered macrocycles containing the thioether bridge via S_{N}Ar methodology. Tetrahedron 54: 10635-10640.

Kiselyov, A., S. Eisenberg and Y. Luo (1999a) Tetrahedron Lett. 40: 2465-2468.

Kiselyov, A., L. Smith, II and P. Tempest (1999b) Solid support synthesis of 14- and 17-membered macrocycles via the S_{N}Ar methodology. Tetrahedron 55: 14813-14822.

Klimstra, W. B., E. M. Nangle, M. S. Smith, A. D. Yurochko and K. D. Ryman (2003) DC-SIGN and L-SIGN can act as attachment receptors for alphaviruses and distinguish between mosquito cell- and mammalian cell-derived viruses. J. Virol. 77: 12022-12032.

Knolle, P. A. and G. Gerken (2000) Local control of the immune response in the liver. Immunol. Rev. 174: 21-34.

Kobayashi, N., H. Taguchi-Nakamura, M. Goto, T. Nakamura, K. Nakamura , W. Sugiura, A. Iwamoto and Y. Kitamura (2002) Polymorphisms and haplotypes of the CD209L gene and their association with the clinical courses of HIV-positive Japanese patients. Jpn. J. Infect. Dis. 55: 131-133.

Kohler, G. and C. Milstein (1975) Continuous cultures of fused cells secreting antibody of predefined specificity. Nature 256: 495-497.

Kolykhalov, A. A., E. V. Agapov, K. J. Blight, K. Mihalik, S. M. Feinstone and C. M. Rice (1997) Transmission of hepatitis C by intrahepatic inoculation with transcribed RNA. Science 277: 570-574.

Kwon, D. S., G. Gregorio, N. Bitton, W. A. Hendrickson and D. R. Littman (2002) DC-SIGN-mediated internalization of HIV is required for trans-enhancement of T cell infection. Immunity 16: 135-144.

Lagging, L. M., K. Meyer, R. J. Owens and R. Ray (1998) Functional role of hepatitis C virus chimeric glycoproteins in the infectivity of pseudotyped virus. J. Virol. 72: 3539-3546.

Laskus, T., M. Radkowski, L. F. Wang, M. Nowicki and J. Rakela (2000) Uneven distribution of hepatitis C virus quasispecies in tissues from subjects with end-stage liver disease: confounding effect of viral adsorption and mounting evidence for the presence of low-level extrahepatic replication. J. Virol. 74: 1014-1017.

Lauer, G. M. and B. D. Walker (2001) Hepatitis C virus infection. New Engl. J. Med. 345: 41-52.

Lavillette, D., A. W. Tarr, C. Voisset, P. Donot, B. Bartosch, C. Bain, A. H. Patel, J. Dubuisson, J. K. Ball and F. L. Cosset (2005) Characterization of host-range and cell entry properties of the major genotypes and subtypes of hepatitis C virus. Hepatology 41: 265-274.

Lechner, F., D. K. Wong, P. R. Dunbar, R. Chapman, R. T. Chung, P. Dohrenwend, G. Robbins, R. Phillips, P. Klenerman and B. D. Walker (2000) Analysis of successful immune responses in persons infected with hepatitis C virus. J. Exp. Med. 191: 1499-1512.

Lee, B., G. Leslie, E. Soilleux, U. O'Doherty, S. Baik, E. Levroney, K. Flummerfelt, W. Swiggard, N. Coleman, M. Malim et al. (2001) cis Expression of DC-SIGN allows for more efficient entry of human and simian immunodeficiency viruses via CD4 and a coreceptor. J. Virol. 75: 12028-12038.

Lichten, M. J. and M. S. Fox (1983) Detection of non-homology-containing heteroduplex molecules. Nucl. Acids Res. 11: 3959-3971.

Liu, H., S. Holte, J. Lee, C. Celum, L. Corey, M. J. McElrath and T. Zhu (2003) Genetic polymorphisms in DC-SIGNR repeat region affect HIV-1 transmission. 10th Conference on Retroviruses and Opportunistic Infections, Abstract 373, Boston, Mass., Feb. 10-14, 2003.

Liu, H., Y. Hwangbo, S. Holte, J. Lee, C. Wang, N. Kaupp, H. Zhu, C. Celum, L. Corey, M. J. McElrath et al. (2004) Analysis of genetic polymorphisms in CCR5, CCR2, stromal cell-derived factor-1, RANTES, and dendritic cell-specific intercellular adhesion molecule-3-grabbing nonintegrin in seronegative individuals repeatedly exposed to HIV-1. J. Infect. Dis. 190: 1055-1058.

Lozach, P. Y., A. Amara, B. Bartosch, J. L. Virelizier, F. Arenzana-Seisdedos, F. L. Cosset and R. Altmeyer (2004) C-type lectins L-SIGN and DC-SIGN capture and transmit infectious hepatitis C virus pseudotype particles. J. Biol. Chem. 279: 32035-32045.

Lozach, P. Y., H. Lortat-Jacob, A. de Lacroix de Lavalette, I. Staropoli, S. Foung et al. (2003) DC-SIGN and L-SIGN are high affinity binding receptors for hepatitis C virus glycoprotein E2. J. Biol. Chem. 278: 20358-20366.

Lu, Y. E. and M. Kielian (2000) Semliki forest virus budding: assay, mechanisms, and cholesterol requirement. J Virol. 74: 7708-7719.

Ludwig, I. S., A. N. Lekkerkerker, E. Depla, F. Bosman, R. J. P. Musters, S. Depraetere, Y. van Kooyk and T. B. H. Geijtenbeek (2004) Hepatitis C virus targets DC-SIGN and L-SIGN to escape lysosomal degradation. J. Virol. 78: 8322-8332.

Martin, M. P., M. M. Lederman, H. B. Hutcheson, J. J. Goedert., G. W. Nelson, Y. van Kooyk, R. Detels, S. Buchbinder, K. Hoots, D. Vlahov et al. (2004) Association of DC-SIGN promoter polymorphism with increased risk for parenteral, but not mucosal, acquisition of human immunodeficiency virus type 1 infection. J. Virol. 78: 14053-14056.

Marzi, A., T. Gramberg, G. Simmons, P. Moller, A. J. Rennekamp, M. Krumbiegel, M. Geier, J. Eisemann, N. Turza, B. Saunier et al. (2004) DC-SIGN and DC-SIGNR interact with the glycoprotein of Marburg virus and the S protein of severe acute respiratory syndrome coronavirus. J. Virol. 78: 12090-12095.

Matsuura, Y., T. Suzuki, R. Suzuki, M. Sato, H. Aizaki, I. Saito and T. Miyamura (1994) Processing of E1and E2 glycoproteins of hepatitis C virus expressed in mammalian and insect cells. Virology 205: 141-150.

McHutchison, J. G., S. C. Gordon, E. R. Schiff, M. L. Shiffman, W. M. Lee et al. (1998) Interferon alpha-2b alone or in combination with ribavirin as initial treatment for chronic hepatitis C. Hepatitis Interventional Therapy Group. New Engl. J. Med. 339: 1485-1492.

McKeating, J. A., L. Q. Zhang, C. Logvinoff, M. Flint, J. Zhang, J. Yu, D. Butera, D. D. Ho, L. B. Dustin, C. M. Rice et al. (2004) Diverse hepatitis C virus glycoproteins mediate viral infection in a CD81-dependent manner. J. Virol. 78: 8496-8505.

Mellman, I. (1996) Endocytosis and molecular sorting. Annual Rev. Cell Dev. Biol. 12: 625.

Meola, A., A. Sbardellati, E. B. Bruni, M. Cerretani, M. Pezzanera et al. (2000) Binding of hepatitis C virus E2 glycoprotein to CD81 does not correlate with species permissiveness to infection. J. Virol. 74: 5933-5938.

Mitchell, D. A., A. J. Fadden and K. Drickamer (2001) A novel mechanism of carbohydrate recognition by the C-type lectins DC-SIGN and DC-SIGNR: Subunit organization and binding to multivalent ligands. J Biol. Chem. 276: 28939-28945.

Mummidi, S., G. Catano, L. Lam, A. Hoefle, V. Telles, K. Begum, F. Jimenez, S. S. Ahuja and S. K. Ahuja (2001) Extensive repertoire of membrane-bound and soluble dendritic cell-specific ICAM-3-grabbing nonintegrin 1 (DC-SIGN1) and DC-SIGN2 isoforms: Inter-individual variation in expression of DC-SIGN transcripts. J. Biol. Chem. 276: 33196-33212.

Orita, M., H. Iwahana, H. Kanazawa, K. Hayashi and T. Sekiya (1989) Detection of polymorphisms of human DNA by gel electrophoresis as single-strand conformation polymorphisms. Proc. Natl. Acad. Sci. USA 86: 2766-2770.

Ouyang, X., N. Tamayo and A. S. Kiselyov (1999a) Solid support synthesis of 2-substituted dibenz[b,f]oxazepin-11(10H)-ones via S_{N}Ar methodology on AMEBA resin. Tetrahedron 55: 2827-2834.

Ouyang, X. and A. S. Kiselyov (1999b) Fast and efficient synthesis of substituted dibenz[b,f]oxazocines on solid support. Tetrahedron 55: 8295-8302.

Ouyang, X. and A. S. Kiselyov (1999c) Novel synthesis of dibenzo[b,g]1,5-oxazocines. Tetrahedron Lett. 40: 5827-5830.

Pessoa, M. G. and T. L. Wright (1997) Hepatitis C infection in transplantation. Clin. Liver Dis. 11: 663-690.

Petracca, R., F. Falugi, G. Galli, N. Norais, D. Rosa et al. (2000) Structure-function analysis of hepatitis C virus envelope-CD81 binding. J. Virol. 74: 4824-4830.

Pileri, P., Y. Uematsu, S. Campagnoli, G. Galli, F. Falugi et al. (1998) Binding of hepatitis C virus to CD81. Science 282: 938-941.

Pohlmann, S., E. J. Soilleux, F. Baribaud, G. J. Leslie, L. S. Morris, J. et al. (2001a) DC-SIGNR, a DC-SIGN homologue expressed in endothelial cells, binds to human and simian immunodeficiency viruses and activates infection in trans. Proc. Natl. Acad. Sci. U.S.A. 98: 2670-2675.

Pohlmann, S., Baribaud, F., Lee, B., Leslie, G. J., Sanchez, M. D., Hiebenthal-Millow, K., Munch, J., Kirchhoff, F. & Doms, R. W. (2001b) DC-SIGN interactions with human immunodeficiency virus type 1 and 2 and simian immunodeficiency virus. J. Virol. 75: 4664-4672.

Pohlmann, S., G. J. Leslie, T. G. Edwards, T. Macfarlan, J. D. Reeves, K. Hiebenthal-Millow, F. Kirchhoff, F. Baribaud and R. W. Doms (2001c) DC-SIGN interactions with human immunodeficiency virus: virus binding and transfer are dissociable functions. J. Virol. 75: 10523-10526.

Pohlmann, S., J. Zhang, F. Baribaud, Z. Chen, G. J. Leslie, G. Lin, A. Granelli-Piperno, R. W. Doms, C. M. Rice and J.A. McKeating (2003) Hepatitis C virus glycoproteins interact with DC-SIGN and DC-SIGNR. J. Virol. 77: 4070-4080.

Rice, C. M. (1996) Flaviviridiae: The viruses and their replication. 3rd ed. In “Fields Virology” (B. N. Fields, Ed.) pp. 931-1034. Lippincott-Raven Publishers, Philadelphia.

Sanders, R. W., E. C. de Jong, C. E. Baldwin, J. H. Schuitemaker, M. L. Kapsenberg and B. Berkhout (2002) Differential transmission of human immunodeficiency virus type 1 by distinct subsets of effector dendritic cells. J. Virol. 76: 7812-7821.

Scarselli, E., H. Ansuini, R. Cerino, R. M. Roccasecca, S. Acali, G. Filocamo, C. Traboni, A. Nicosia, R. Cortese and A. Vitelli (2002) The human scavenger receptor class B type I is a novel candidate receptor for the hepatitis C virus. EMBO J. 21: 5017-5025.

Schagger, H. and G. von Jagow (1991) Blue native electrophoresis for isolation of membrane protein complexes in enzymatically active form. Anal. Biochem. 199: 223-231.

Schagger, H., W. A. Cramer and G. von Jagow (1994) Analysis of molecular masses and oligomeric states of protein complexes by blue native electrophoresis and isolation of membrane protein complexes by two-dimensional native electrophoresis. Anal. Biochem. 217: 220-230.

Schulke, N., M. S. Vesanen, R. W. Sanders, P. Zhu, M. Lu, D. J. Anselma, A. R. Villa, P. W. Parren, J. M. Binley, K. H. Roux, P. J. Maddon, J. P. Moore and W. C. Olson (2002) Oligomeric and conformational properties of a proteolytically mature, disulfide-stabilized human immunodeficiency virus type 1 gp140 envelope glycoprotein. J. Virol. 76: 7760-7776.

Serrano-Gomez, D., A. Dominguez-Soto, J. Ancochea, J. A. Jimenez-Heffernan, J. A. Leal and A. L. Corbi (2004) Dendritic cell-specific intercellular adhesion molecule 3-grabbing nonintegrin mediates binding and internalization of Aspergillus fumigatus conidia by dendritic cells and macrophages. J. Immunol. 173: 5635-5643.

Sieczkarski, S. B. and G. R. Whittaker (2002) Dissecting virus entry via endocytosis. J. Gen. Virol. 83: 1535-1545.

Smith, D. B. and P. Simmonds (1998) Hepatitis C virus: types, subtypes and beyond. In “Methods in Molecular Medicine” (Lau, ed.), pp. 134-146. Humana Press, Totowa.

Soilleux, E. J., R. Barten and J. Trowsdale (2000) DC-SIGN; a related gene, DC-SIGNR; and CD23 form a cluster on 19p13. J. Immunol. 165: 2937-2942.

Spaete, R. R., D. Alexander, M. E. Rugroden, Q. L. Choo, K. Berger et al. (1992) Characterization of the hepatitis C virus E2/NS1 gene product expressed in mammalian cells. Virology 188: 819-830.

Takikawa, S., K. Ishii, H. Aizaki, T. Suzuki, H. Asakura, Y. Matsuura and T. Miyamura (2000) Cell fusion activity of hepatitis C virus envelope proteins. J. Virol. 74: 5066-5074.

Tassaneetrithep, B., T. H. Burgess, A. Granelli-Piperno, C. Trumpfheller, J. Finke, W. Sun, M. A. Eller, K. Pattanapanyasat, S. Sarasombath, D. L. Birx et al. (2003) DC-SIGN (CD209) mediates dengue virus infection of human dendritic cells. J. Exp. Med. 197: 823-829.

Trkola, A., T. Dragic, J. Arthos, J. M. Binley, W. C. Olson et al. (1996) CD4-dependent, antibody-sensitive interactions between HIV-1 and its co-receptor CCR-5. Nature 384: 184-187.

Van Kooyk, Y. and T. B. Geijtenbeek (2003) DC-SIGN: escape mechanism for pathogens. Nat. Rev. Immunol. 3: 697-709.

Voisset, C., N. Callens, E. Blanchard, A. Op De Beeck, J. Dubuisson and N. Vu-Dac (2005) J. Biol. Chem. 280(9): 7793-7799.

Wang, D. G., J. B. Fan, C. J. Siao, A. Berno, P. Young et al. (1998) Large-scale identification, mapping, and genotyping of single-nucleotide polymorphisms in the human genome. Science 280: 1077-1082.

Wei, G. P. and G. B. Phillips (1998) Solid phase synthesis of benzimidazolones. Tetrahedron Lett. 39: 179-182.

Wu, L., T. D. Martin, M. Carrington and V. N. KewalRamani (2004) Raji B cells, misidentified as THP-1 cells, stimulate DC-SIGN-mediated HIV transmission. Virology 318: 17-23.

Wu, L., T. D. Martin, R. Vazeux, D. Unutmaz and V. N. KewalRamani (2002) Functional evaluation of DC-SIGN monoclonal antibodies reveals DC-SIGN interactions with ICAM-3 do not promote human immunodeficiency virus type 1 transmission. J. Virol. 76:5905-5914.

Wunschmann, S., J. D. Medh, D. Klinzmann, W. N. Schmidt and J. T. Stapleton (2000) Characterization of hepatitis C virus (HCV) and HCV E2 interactions with CD81 and the low-density lipoprotein receptor. J. Virol. 74: 10055-10062.

Yanagi, M., R. H. Purcell, S. U. Emerson and J. Bukh (1999) Hepatitis C virus: an infectious molecular clone of a second major genotype (2a) and lack of viability of intertypic 1a and 2a chimeras. Virology 262: 250-263.

Yang, Z. Y., Y. Huang, L. Ganesh, K. Leung, W. P. Kong, O. Schwartz, K. Subbarao and G. J. Nabel (2004) pH-dependent entry of severe acute respiratory syndrome coronavirus is mediated by the spike glycoprotein and enhanced by dendritic cell transfer through DC-SIGN. J. Virol. 78: 5642-5650.

1. A method for determining whether an agent preferentially binds to at least one allelic variant of L-SIGN, comprising:
(a) separately contacting an agent with one of (i) at least two allelic variants of L-SIGN, (ii) at least two cell lines expressing allelic variants of L-SIGN, and (iii) plasma membrane fractions from extracts of said at least two cell lines, under conditions suitable for binding of the agent; and
(b) comparing the relative binding of the agent to the allelic variants, or to the cells or membrane fractions expressing allelic variants with which the agent is contacted, wherein a difference in relative binding indicates that the agent preferentially binds to at least one allelic variant of L-SIGN.

2-5. (canceled)

6. A method for identifying a monoclonal antibody that specifically binds to an allelic variant of L-SIGN, comprising:
(a) administering to a subject an allelic L-SIGN variant protein or an expression vector comprising a nucleic acid which encodes said allelic L-SIGN variant protein;
(b) harvesting antibody-producing lymphatic cells from the subject;
(c) generating hybridomas by fusing single antibody-producing cells obtained in step (b) with myeloma cells; and
(d) screening hybridoma supernatants from said hybridomas by the agent-screening method of claim 1 to identify a monoclonal antibody that specifically binds to the allelic variant of L-SIGN.

7. An agent that preferentially binds at least one allelic variant of L-SIGN.

8. The agent of claim 7, wherein the agent preferentially binds at least one disease-associated allelic variant with at least 2 times greater avidity than it binds at least one non-disease-associated allelic variant.

9. The agent of claim 7, wherein the agent preferentially binds at least one disease-associated allelic variant with at least 5 times greater avidity than it binds at least one non-disease-associated allelic variant.

10. The agent of claim 7, wherein the agent preferentially binds at least one disease-associated allelic variant with at least 10 times greater avidity than it binds at least one non-disease-associated allelic variant.

11. The agent of claim 8, wherein the at least one disease-associated allelic variant is L-SIGN-7 or L-SIGN-9.

12. The agent of claim 8, wherein the at least one non-disease-associated allelic variant is any of L-SIGN-3, L-SIGN-4 or L-SIGN-5.

13. The agent of claim 12, wherein the at least one non-disease-associated allelic variant is L-SIGN-3.

14. The agent of claim 7, wherein the agent is an antibody or fragment thereof.

15. The agent of claim 14, wherein the antibody is a monoclonal antibody.

16. The agent of claim 14, wherein the fragment of the antibody is a fragment of a monoclonal antibody.

17-30. (canceled)

31. The agent of claim 7, wherein the agent is a peptide.

32. The agent of claim 7, wherein the agent comprises a peptide bond.

33. The agent of claim 7, wherein the agent is a non-peptidyl agent.

34. The agent of claim 33, wherein the non-peptidyl agent is a carbohydrate.

35. The agent of claim 34, wherein the carbohydrate is mannose, mannan or methyl-D-mannopyranoside.

36. The agent of claim 7, wherein the agent is a small molecule or low molecular weight molecule.

37. The agent of claim 36, wherein the molecule has a molecular weight less than 500 daltons.

38-42. (canceled)

43. The agent of claim 7, wherein the agent preferentially binds at least one non-disease-associated allelic variant with at least 2 times greater avidity than it binds at least one disease-associated allelic variant.

44-45. (canceled)

46. The agent of claim 43, wherein the at least one non-disease-associated allelic variant is any of L-SIGN-3, L-SIGN-4 or L-SIGN-5.

47. The agent of claim 46, wherein the at least one non-disease-associated allelic variant is L-SIGN-3.

48. The agent of claim 43, wherein the agent is an antibody or fragment thereof.

49. The agent of claim 48, wherein the antibody is a monoclonal antibody.

50. The agent of claim 48, wherein the fragment of the antibody is a fragment of a monoclonal antibody.

51. A composition comprising the agent of claim 7 and a carrier.

52-53. (canceled)

54. A method for treating a subject afflicted with a pathogen-related disorder, susceptibility to which is associated with at least one polymorphism in L-SIGN, which method comprises administering to the subject an agent, wherein
(1) the agent is determined to preferentially bind to at least one allelic variant of L-SIGN using the method of claim 1 and
(2) the agent is administered to the subject in a therapeutically effective amount to treat the subject.

55-56. (canceled)

57. A method for preventing infection of a subject by a pathogen, susceptibility to which infection is associated with at least one polymorphism in L-SIGN, which method comprises administering to the subject an agent, wherein
(1) the agent is determined to preferentially bind to at least one allelic variant of L-SIGN using the method of claim 1 and
(2) the agent is administered to the subject in a prophylactically effective amount to prevent infection by the pathogen.

58-59. (canceled)

60. A method for inhibiting in a subject the onset of a pathogen-related disorder, susceptibility to which is associated with at least one polymorphism in L-SIGN, which method comprises administering to the subject an agent, wherein
(1) the agent is determined to preferentially bind to at least one allelic variant of L-SIGN using the mthod of claim 1 and
(2) the agent is administered to the subject in a prophylactically effective amount to have a prophylactic effect in the subject.

61-67. (canceled)

68. A method for predicting resistance of a subject to infection by a pathogen by determining the status of L-SIGN Exon 4 repeat polymorphisms in the subject and correlating that status to a degree of resistance of the subject to said pathogen, which method comprises:
(a) amplifying genomic DNA from cells of the subject by a polymerase chain reaction (PCR) using primers that are specific for Exon 4 of L-SIGN;
(b) identifying the L-SIGN alleles present by determining the size of the amplified DNA, wherein the size of the amplified DNA is proportional to the number of Exon 4 repeats in the allele; and
(c) correlating the identity of said L-SIGN alleles in the subject with allelic combinations known to be associated with resistance to infection by the pathogen.

69. The method of claim 68, wherein the L-SIGN alleles present in the subject comprise L-SIGN-3, L-SIGN-4 or L-SIGN-5 alleles, or a combination thereof.

70. The method of claim 69, wherein the L-SIGN alleles present in the subject are L-SIGN-3 alleles.

71. The method of claim 68, wherein the pathogen is selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species.

72. The method of claim 71, wherein the pathogen is hepatitis C virus (HCV).

73-74. (canceled)

75. A method for predicting susceptibility of a subject to infection by a pathogen by determining the status of L-SIGN Exon 4 repeat polymorphisms in the subject and correlating that status to a degree of susceptibility of the subject to said pathogen, which method comprises:
(a) amplifying genomic DNA from cells of the subject by a polymerase chain reaction (PCR) using primers that are specific for Exon 4 of L-SIGN;
(b) identifying the L-SIGN alleles present by determining the size of the amplified DNA, wherein the size of the amplified DNA is proportional to the number of Exon 4 repeats in the allele; and
(c) correlating the identity of said L-SIGN alleles in the subject with allelic combinations known to be associated with susceptibility to infection by the pathogen.

76. The method of claim 75, wherein the L-SIGN alleles present in the subject are L-SIGN-7 or L-SIGN-9 alleles, or a combination thereof.

77. The method of claim 75, wherein the pathogen is selected from the group consisting of hepatitis C virus (HCV), simian immunodeficiency virus (SIV), dengue virus, Ebola virus, Marburg virus, severe acute respiratory syndrome (SARS) coronavirus, Sindbis virus, cytomegalovirus (CMV), Aspergillus, Candida, Mycobacterium, Helicobacter, Leishmania, Schistosoma and sporozoites from Plasmodium species.

78. The method of claim 77, wherein the pathogen is hepatitis C virus (HCV).

79-109. (canceled)
